(19) United States
(12) Patent Application Publication
(10) Pub. No.: US 2025/0261894 A1
(43) Pub. Date: Aug. 21, 2025

(54) MONOLITHIC THREE-DIMENSIONAL NEURAL PROBES

(71) Applicant: Trustees of Dartmouth College, Hanover, NH (US)

(72) Inventors: Hui FANG, Hanover, NH (US); Wen GU, Shanghai (CN); Yi QIANG, West Lebanon, NH (US); Kyung Jin SEO, Lebanon, NH (US)

(21) Appl. No.: 19/058,657

(22) Filed: Feb. 20, 2025

Related U.S. Application Data
(60) Provisional application No. 63/555,514, filed on Feb. 20, 2024.

(51) Int. Cl. A61B 5/293; A61B 5/00; A61B 5/268

(52) U.S. Cl. CPC A61B 5/293; A61B 5/268; A61B 5/6868

(57) ABSTRACT

A rolling-of-soft-electronics (ROSE) having a planar component with one or more shanks and one or more electrodes disposed on the one or more shanks. The one or more shanks are positioned in rows and separated by a space having a pitch. The planar component is made of a flexible material that is rolled in a ROSE method such that the one or more shanks are circularly arranged in three-dimensions.

### CROSS REFERENCE TO RELATED APPLICATIONS

This application claims priority to U.S. Provisional Patent Application No. 63/555,514, filed on Feb. 20, 2024, the entire disclosure of which is incorporated herein by reference.

### STATEMENT REGARDING FEDERALLY-SPONSORED RESEARCH OR DEVELOPMENT

This invention was made with government support under contracts R21 EY030710 and U01 NS123668 awarded by the National Institutes of Health. The government has certain rights in the invention.

### FIELD OF THE DISCLOSURE

This disclosure relates to neural probes.

### BACKGROUND OF THE DISCLOSURE

The brain is comprised of interconnected networks over a large range of length scales that consists of 3D spatially-distributed and functionally-linked regions. These 3D spatially-distributed and functionally-linked regions continuously communicate with each other. Because of this, fully decoding cognition of the brain and underlying brain functions requires acquiring neural activity with high spatiotemporal resolution across three dimensions.

Over the past few decades, single-unit recording has been the gold standard for measuring neuronal activity. Specifically, penetrating neural probes serve as the primary tools for measuring single unit activities. Silicon (Si) probes have become the favored electrophysiological technology, primarily due to their high reliability and reasonable costs, and the fact that their properties originated from the mature and scalable manufacturing from the six-decade-long semiconductor industry. Because semiconductor fabrication is a planar process, monolithic Si probes are typically constrained to 2-dimensional (2D) interfacing with the brain. Oftentimes, this structure results in a dimensional mismatch with the 3D brain. For example, when implanted in the brain, multi-shank Michigan-type probes typically contain only a planar electrode array that is orthogonal to the cortical surface. Further, even micromachined Utah-type probes possess a planar array parallel to the cortical surface.

Recent endeavors have focused on achieving a 3D system by stacking multiple individual planar probes for both Si and polymer probes. However, due to the dominating planar semiconductor fabrication techniques, it has been difficult to implement 3D neural probes that are monolithic and achieve a similar scalability as modular Si probes. Cognitive and behavior studies rely on coordinated activity from neural circuits distributed in three dimensions, and current neural probes are constrained to 2D interfacing with the brain.

Therefore, improved systems and methods are needed.

### SUMMARY OF THE DISCLOSURE

The present disclosure provides planar soft electronics directly transformed into monolithic 3D neural ROSE probes by deterministic rolling (ROSE method). Previous techniques focused on achieving a 3D system by stacking multiple individual planar probes for both silicon and polymer probes. However, it has been challenging to develop three-dimensional neural probes that are monolithic and achieve similar scalability as modular silicon probes. Embodiments of the present disclosure, aim to solve both the dimensional mismatch and the mechanical mismatch between neural probes and brains by its inherent softness and monolithic structure.

Embodiments of the present invention include a rolling-of-soft-electronics (ROSE) method to achieving monolithic 3D neural probes (ROSE probes) with high scalability and design flexibility. Compared to previous 3D stacking or assembly methods, the ROSE method directly transforms a planar probe into a 3D monolithic ROSE probe by leveraging the softness of flexible electrodes. The ROSE method is a simple and non-destructive post process. In addition, the ROSE method inherently leverages flexible probes, which reduces the mechanical mismatch between the implanted ROSE probes and surrounding brain tissue when compared to rigid Si probes, a factor known to contribute to probe failure from a complex progression of immune response to neuron death.

The insertion dynamics of ROSE probes of hundreds of electrodes were studied. In addition to demonstrating compelling single-unit spike recording yields in vivo in both rodent and non-human primate models, ROSE probe also revealed microscopy-like 3D spatiotemporal mapping of spike activities in the visual cortex, with spike yield over 60% achieved. ROSE offers general applicability in soft bio-electronics and provides a pragmatic pathway to establish 3D neural probes.

Embodiments of the present disclosure utilize a simple and non-destructive post process.

In an embodiment of the present disclosure, ROSE inherently leverages flexible probes, thereby reducing the mechanical mismatch caused by rigid Si probes, which results in probe failure from a complex progression of immune response to neuron death.

Embodiments of the present disclosure, such as the 3D neural ROSE probes described herein, address a critical unmet need in neural interfaces and establish the ROSE method and its underline design guidelines, which may be applicable to soft bioelectronics.

The present disclosure provides a rolling-of-soft-electronics (ROSE) probe having a planar component with one or more shanks and one or more electrodes disposed on the one or more shanks. In embodiments, the one or more shanks are positioned in rows and separated by a space having a pitch. Further, in embodiments, the planar component may be made of a flexible material. Even further, the planar component may be configured to be rolled.

In an aspect of the present disclosure, the planar component may further include at least one substrate layer and at least one encapsulation layer.

In an aspect of the present disclosure, the planar component may be rolled such that the one or more shanks are circularly arranged in three-dimensions.

In an aspect of the present disclosure, the planar component may have a thickness from 1-100 μm.

In an aspect of the present disclosure, the one or more shanks may have a tapered profile with a tip angle of 20°.

In an aspect of the present disclosure, the one or more shanks may have a width from 10-1,000 μm.

In an aspect of the present disclosure, each of the one or more shanks may have a length from 0.5-5 mm.

In an aspect of the present disclosure, each of the one or more shanks may have a thickness from 2-100 μm.

In an aspect of the present disclosure, the ROSE probe may include from 10-1,000 shanks.

In an aspect of the present disclosure, from 1 to 256 electrodes are disposed on each of the one or more shanks.

In an aspect of the present disclosure, the ROSE probe may further include a spacer layer disposed on a base of the planar component and configured to act as a spacer between the rows.

In an aspect of the present disclosure, the spacer layer may be a polydimethylsiloxane (PDMS) layer.

In an aspect of the present disclosure, the spacer layer may have a thickness from 100-1,000 μm.

In an aspect of the present disclosure, the ROSE probe may further include a connector pad matrix which includes interconnects of the one or more electrodes.

The present disclosure further provides a method of using the ROSE probe as a neural probe.

The present disclosure even further provides a rolling-of-soft-electronics (ROSE) method including providing a planar component having one or more shanks and one or more electrodes disposed on the one or more shanks, fixing a micro-mandrel to the edge of the planar component, and rolling the planar component so that the one or more shanks are circularly arranged in three-dimensions to generate a ROSE probe.

In an aspect of the present disclosure, the planar component includes an adhesive portion configured to be secured around the ROSE probe.

In an aspect of the present disclosure, the one or more shanks may include a tapered profile with a tip angle of 20°.

In an aspect of the present disclosure the ROSE probe may include from 10-1,000 shanks.

In an aspect of the present disclosure, from 1 to 256 electrodes may be disposed on each of the one or more shanks.

### BRIEF DESCRIPTION OF THE FIGURES

For a fuller understanding of the nature and objects of the disclosure, reference should be made to the following detailed description taken in conjunction with the accompanying figures.

FIG. 1A displays a schematic of a monolithic 3D neural probe (ROSE probe), with its final structure of ROSE probe (top right), and the explosive view of a shank structure schematic (bottom right).

FIG. 1B displays an optical image of a ROSE probe with 64 shanks and 256 electrodes. Inset: magnified view of the electrode shanks.

FIGS. 1C-1D display SEM images of the ROSE probe shown in FIG. 1B. Inset: magnified view of an individual shank which has 4 microelectrodes.

FIG. 1E displays an impedance histograms of a 64-shank ROSE probe (256-ch) before and after rolling with a 200-μm-thick PDMS spacer.

FIG. 1F displays a 3D impedance greyscale colormap of the ROSE probe in FIG. 1E, with the Z axis being the electrode number (from bottom to top).

FIG. 2A displays a schematic illustration of the ROSE probe showing the shank pitch and a PDMS spacer layer.

FIGS. 2B-2C display optical images of an embodiment of a 256-ch, 64-shank ROSE probe with versatile shank pitches and PDMS thicknesses.

FIG. 2D displays optical images of an embodiment of a 256-ch ROSE probe with custom shank design, which includes 8, 4, 2 and 1 electrode per shank, respectively. All probes are rolled with 200-μm thick PDMS spacers. Insets: detailed microscope images of individual shanks (top left) and top views (with respect to the perspective in a) of the probes (bottom right). Scale bars: 200 μm for individual shanks and 500 μm for the top views.

FIGS. 2E-2G display comparisons of the rolling loops and offsets between experimental and theoretical results from rolling using different shank pitch (FIG. 2E), PDMS spacer thickness (FIG. 2F), and different shank design/number (FIG. 2G) for the 256-ch ROSE probes.

FIG. 2H displays maximum principal strain contour (εₘₐₓ) of the electrodes shown in three individual shanks (left) and in the interconnect layer within the rolled planar component base (right). The left shows three individual shanks (Shank No. 1, 32, and 64) in a same 64-shank ROSE probe.

FIG. 2I displays the average electrode impedance and probe yield comparison of the same device before and after rolling with different PDMS spacer thicknesses.

FIG. 3A display insertion force dynamics of a 64-shank, 256-ch ROSE probe with a 300-μm thick PDMS spacer and a 300-μm shank pitch. Inset images are the real-time photos of the probe at different insertion stage. The concentration of the agarose gel was 0.6% and the insertion speed was 0.5 mm/min. Scale bar: 1 mm.

FIG. 3B displays the comparison of insertion force dynamics between a 64-shank planar probe (110-μm shank width, 300-μm shank pitch) attached on a glass slide and a 3D ROSE probe from the same planar probe design (rolled with a 200-μm thick PDMS spacer).

FIGS. 3C-3D display comparisons of the puncture forces between experimental and theoretical results from rolling using different PDMS spacer thickness (FIG. 3C) and shank pitch (FIG. 3D).

FIG. 3E displays a schematic illustration for pre-puncturing stage of a ROSE probe, highlighting both successful shank puncturing into the gel and shank-slipping-induced failure if the force before puncturing is too large.

FIG. 3F displays the scaling law of the ROSE-probe insertion considering slipping with different PDMS spacer thicknesses and shank pitches compared to experimental observations. The dashed curve is calculated by the equation F_{c}(a,b)=Fₜₒₜₐₗ(w,d,fₛ), where F_{c} is the critical contact force for slipping with inner diameter a and outer diameter b of a hollow cylinder, Fₜₒₜₐₗ is the total insertion force of the probes with shank pitch w, PDMS spacer d, and the puncture force for a single shank fₛ. Diamond (indicating failure) and circle (indicating success) points are from experimental observation.

FIG. 3G displays the average electrode impedance and probe yield of a ROSE probe (300-μm thick PDMS spacer and 300-μm shank pitch) as a function of insertion cycles without any insertion aids.

FIG. 4A left displays an illustration of a ROSE probe (32 shanks, 4 electrodes per shank) implanted into a rat visual cortex. FIG. 4A right displays photographs of different stages in the insertion process of the ROSE probe. Scale bar: 0.5 mm.

FIG. 4B displays broad-band neural signals (0.1-7000 Hz) recorded from a ROSE probe. Representative data from one shank are shown, and the layout of the single shank design is displayed on the left with electrodes labeled A to D from the tip of the shank. SU spikes were detected and sorted, 100 spike waveforms for each unit are overlaid on the right (lines represent the averaged spike waveform of each SU).

FIG. 4C displays spontaneous spike firing rate at multiple recording depths (Layer II/III to Layer VI, across 5 animals). Data shown: mean and standard deviation (SD).

FIG. 4D displays representative LFP spectrogram and spike raster plot of a 3-s recording from 2 trials of visual stimulation, demonstrating a 66% SU yield. Data from 64 channels exhibited significantly higher high-gamma power and increased spike firing rates (p<0.05, Student's paired t-test, N=180 trials).

FIG. 4E displays spatial-temporal spike firing rate mapping in Layer II/III of rat visual cortex, using 3D ROSE probe configuration by averaging data collected in 180 trials of visual stimulation. Subcortical regions in the visual cortex are labeled with shadings, showing the transmission of visually evoked responses from V1m to other regions. The visual stimuli started at t=0 ms.

FIG. 4F displays SU yield across 5 weeks, 5 animals, which is defined as the number of electrodes that recorded at least 1 identifiable SU divided by the initial number of functional electrodes at week 0.

FIG. 4G displays spike amplitudes recorded from one representative mouse across 5 weeks. The box is determined by the 25th and 75th percentiles of data while the whiskers indicate 1.5 times the interquartile range. The asterisks represent the minimum and maximum values while the horizontal lines and small squares within the box indicate the median and mean, respectively. No significant differences in spike amplitudes are observed across 5 weeks of recordings (′ns'=non-significant, p=0.11, One-way ANOVA, N=54-68 units in each week's recording). The same analysis was applied to each mice's data and showed no significant differences in recorded spike amplitudes across 5 weeks (p>0.05 for 5 mice).

FIG. 4H displays unit similarity (Mahalanobis distances between the centroids of clusters) calculated for all unit pairs within weeks (gray) and within the identified same units, across weeks (orange) from one representative mouse, using a threshold=5th percentile of the entire distribution (DM=1.05). The 5th percentile of the within-week unit similarity consistently exceeded 1 (p<0.05, one-sided binomial test with Bonferroni correction, N=30 recording sessions), indicating a good isolation of single units in each recording.

FIG. 4I displays representative SU tracking in the 5 weeks of recording. 4 example SUs that are stable across 5 weeks are shown (150 waveforms per week; white lines represent the averaged spike waveform). The unit similarity within each unit, across weeks, ranges from 0.3-0.98, below a 5th percentile of distribution=1.05, aligning with their consistent waveforms.

FIG. 4J displays representative histology images of the tissue-device interface from ROSE probe and traditional planar Si probe 4 weeks post-implantation. The neuronal marker (NeuN) shows that the neuron density around the probe hole is comparable between flexible ROSE and stiff Si. The microglia (Iba-1) marker shows less microglia activation around ROSE probe hole than the stiff Si probe hole. Scale bar: 100 μm. The asterisk marks the center of the probe hole.

FIG. 4K displays a quantitative tissue fluorescent intensity analysis. 10-μm wide concentric bins that centered on the probe hole are created, and tissue markers are analyzed within each bin and plotted as a function of distance to the hole. Manually counted neuron density in ROSE group is overall statistically similar to the Si group (p=0.8064). Normalized Iba-1 intensity shows that ROSE probe has overall significantly less microglia activation (p<0.0001, N=13 images for ROSE, 37 images for Si probes), especially within 0-50 μm region from the probe hole. “ns”=not significant, ****p<0.0001. Two-way ANOVA with Sidak's multiple comparisons. Data are shown as the mean and standard error of the mean (MSE).

FIG. 4L displays broad-band neural signals (0.1-9000 Hz) recorded from the ROSE probe. Representative data from 2 shanks are shown and the layout of the single shank design is displayed on the left with electrodes labeled from A to D from the tip of the shank. Single-unit spikes were detected and sorted, shown on the right (Black lines show the averaged spike waveform of each single unit).

FIG. 4M displays spike firing rate at 4 different insertion depth (electrode A-D). Electrodes in position ‘D’ show the highest spike firing rate. Data points are the averaged firing rate from all the electrodes at each insertion depth.

FIG. 4N displays LFP spectrogram and spike raster plot of a 9-s recording with 2 trials of visual stimulations included, demonstrating 59% single-unit spike recording.

FIG. 4O displays a peristimulus time histogram (PSTH) of average spike firing rate in all 113 functioning channels during 4 different trials of visual stimulation. The dashed line shows the time period of visual stimuli.

FIG. 5A displays an illustration of a 32-shank, 256-ch ROSE probe implanted in a macaque monkey's visual cortex (V4/IPL area). Different shadings are used to identify 32 shanks. Inset on the top right indicates the probe's placement within brain, while the bottom right inset details the single-shank layout, with electrodes labeled from A to H.

FIG. 5B displays example raw data recorded from monkey brain, displaying 50 seconds data from 24 channels (1 row per channel) extracted from 3 shanks (8 channel per shank, shown from shank tip to bottom).

FIG. 5C upper displays laminar evoked LFP from one representative shank. Black dashed line denotes the onset of visual stimulation. FIG. 5C lower displays current source density (CSD) laminar profile derived from the same data. Negative values indicates extracellular current sinks and positive values indicates extracellular current sources. Evoked LFP responses from electrode ‘B’ and ‘E’ (locations corresponding to observed current sinks) are superimposed on the CSD plots for reference.

FIG. 5D displays 2D CSD plots from a single electrode plane (layer ‘E’), where the evoked LFP responses peak. Averaged LFP data 1s before (left) and 1s after (right) visual stimulation was extracted from 32 electrodes, then interpolated onto a 50×50 grid for the data visualization. The CSD plots clearly shows the local current flow patterns from the outer shanks towards the inner shanks at the specific plane. Gray dots denote the physical location of each shank in the electrode plane.

FIG. 5E displays auto-correlogram and cross-correlograms for 8 example SUs, plotted over a −75 to +75 ms window (bin size=1 ms), showing diverse SU spiking patterns in a large-scale 3D recording. 150 spike waveforms per unit are overlaid, with the white line representing the average spike waveform. A 3D spike firing rate map (bottom left) visualizes captured SUs and their firing rates over a 2-minute recording window, with asterisks marking the locations of the 8 example SUs.

FIG. 5F displays spiking raster from 2 trials of visual stimulation, with spiking activity from each individual shank coded with same shading. Dashed lines denote the ‘on’ and ‘off’ times of visual stimuli.

FIG. 5G displays 70 representatives, sorted single-unit spikes recorded from 23 shanks in the monkey brain.

FIG. 6A displays schematic procedures for the ROSE method. A Kapton tape is attached on the back of the sample edge before rolling. After the final rolling, it can be used to secure the sample. Medical tissue adhesive (3M Vetbond) was used to glue the micro-mandrel onto the sample.

FIG. 6B displays photos of a 3D ROSE probe after micro-mandrel removal. The micro-mandrel removal method includes dipping the sample into acetone and clean with ultrasonic washer for 20 seconds, then the micro-mandrel will be automatically separated with the sample.

FIG. 7A displays a schematic illustration of the planar membrane electrode array (MEA) fabrication process (ROSE method).

FIG. 7B displays a photo of a planar 64-shank planar membrane electrode array on glass/PDMS substrate. Inset is the zoom in picture of the shanks (4 electrodes per shank, 300 μm shank pitch).

FIG. 8A displays micromotion-induced strain simulation for ROSE and Si probes, with an illustration of the probe geometry showing a section of the ROSE probe that contains 4 shanks, which is a subset of a 64-shank ROSE probe. Both the top and isometric views are illustrated.

FIG. 8B displays an illustration of the simulation setup with boundary conditions. The probe base is fixed, and a 6 μm displacement is applied to mimic the effect of micromotion. Three different view planes are also shown to visualize the strain field.

FIG. 8C displays a visualization of the micromotion-induced strain distribution around a 4-shank ROSE probe made of Kapton from two different views.

FIG. 8D displays a visualization of the micro-motion-induced strain distribution around a 4-shank ROSE probe made of silicon from two different views. Regions where the strain exceeds 5% are depicted.

FIG. 9A displays a schematic illustration of the recording connector configured with a 3D ROSE probe.

FIG. 9B displays a maximum principal strain contour on the pad area of the probe with magnet configurations (by finite element analysis, FEA).

FIG. 9C displays contact resistance.

FIG. 9D displays crosstalk coefficient mappings of the 256 channels in the connector (by measurements).

FIG. 10A displays an impedance magnitude and phase spectra of a representative channel in an embodiment of a 64-shank ROSE probe.

FIG. 10B displays a bench recording output of 1 kHz sine wive input in an embodiment of a 64-shank ROSE probe.

FIG. 10C displays a power spectra density of a representative channel with 300-6000 Hz bandpass filter in an embodiment of a 64-shank ROSE probe.

FIG. 10D displays a SNR histogram of a in an embodiment of a 64-shank ROSE probe. Test signal: 110μ Vᵣₘₛ, 1 kHz Sinewave signal.

FIG. 11 displays impedance greyscale colormaps on the pads area of the 256-ch connector with the same ROSE probe device before and after rolling (64 shanks, 300-μm shank pitch, rolled with 200-μm thick PDMS spacer).

FIG. 12 displays microscope images of planar probes as disclosed herein with different shank pitch designs.

FIGS. 13A-13C display embodiments of ROSE probes with varying shank lengths. For example, 0.9 mm (FIG. 13A), 2.5 mm (FIG. 13B), 0.9-2.5 mm (FIG. 13C).

FIG. 14A displays the calculation method of X and Y offsets by using an embodiment of a 32-shank ROSE probe. Procedure: 1. Mark the shanks with circles by actual top view image of the device and create temporary coordinates for them (left). 2. Create the hollow circles of the shank positions by theoretical calculation (middle). 3. Put the experimental and theoretical results into a same coordinate system, overlay the most outer shank position first and then tune the angle of the theoretical result to have the best overlap with the actual result (right). 4. Calculate the X and Y differences between the experimental and theoretical result for all the shanks. Scale bars: 500 μm.

FIG. 14B displays X and Y offsets of the 32-shank ROSE probe.

FIG. 14C displays normal distribution of the X and Y offsets.

FIG. 14D displays the average X and Y offsets and their corresponding root mean squared errors, showing the very small difference (less than 30 μm in average) between the theoretical and actual results.

FIG. 15A displays the maximum principal strain contour of Kapton, PI, electrode and SU-8 layers for the most inner shank (1st) in 64-shank ROSE probe (300 μm shank pitch and 200 μm thick PDMS spacer).

FIG. 15B displays the 2D positions of the inner (1st), middle (32nd) and outer (64th) shanks in 64-shank planar MEA.

FIG. 15C displays the top view positions of the inner, middle and outer shanks in the 64-shank ROSE probe.

FIG. 16A displays a FEA analysis of reliability of ROSE neck with an illustration and boundary conditions of bending simulation.

FIG. 16B displays an illustration and boundary conditions of a twisting simulation.

FIG. 16C displays strain distribution under bending at bending radius of 10.73 mm and 1.12 mm.

FIG. 16D displays strain distribution under twisting at 90° and 180°. Scalebar: 5 mm. Table: Maximum strain in the device under bending and twisting by FEA modeling.

FIG. 17A displays a cycling bending test of the “neck” of the ROSE probe.

FIG. 17B displays impedance and yield change in cycling bending tests up to 10000 cycles.

FIG. 18 displays device yield and average impedance (at 1 kHz) of ROSE probes (N=4) after being soaked in a PBS solution for an accelerated aging test at 77° C. for 10 days (equivalent to 160 days at 37° C.). Data: mean±SD.

FIG. 19A displays a gel insertion test setup and an optical image of Mark-10 force test stand with mN range force gauge. The ROSE probe was clamped under the gauge and 0.6% agarose gel was on the base plate.

FIG. 19B displays a zoomed in photo of the ROSE probe and Agarose gel.

FIG. 20 displays the corresponding insertion displacement versus time of the demonstrated sample in FIG. 3A. The insertion retraction speed was 0.5 mm/min. The insertion stopped at 2-minute after the gel contact with a final displacement of 1 mm.

FIG. 21A displays the insertion force dynamics of 64-shank ROSE probes with different shank pitches (200, 300, 400, and 500 μm) while the PDMS spacer thickness was fixed at 200 μm.

FIG. 21B displays the insertion force dynamics of 64-shank ROSE probes with 100, 200, 300, and 400 μm thick PDMS spacers while the shank pitch was fixed at 300 μm.

FIG. 22A displays insertion force dynamics of a single shank. The puncture force is around 0.46 mN. The minimum resolution of the force gauge is 0.1 mN. Due to the limitation of the gauge, another probe with 10 shanks (900-μm shank pitch with widely separated shanks to avoid interactions among shanks) was used to obtain more accurate results. An insertion test was tried 10 times with the probe and the average value of the total puncture force was 4.6 mN (0.46 mN for a single shank).

FIG. 22B displays a typical insertion force dynamics of the 10-shank planar probe.

FIG. 23A displays a cyclic gel insertion test setup overview of the cyclic insertion test with a ROSE probe and agarose gel (0.6%).

FIG. 23B displays a different insertion step from one cycle, including approaching, contacting, deep insertion and retracting. The insertion speed was 0.5 mm/s, each cycle took 8 seconds. The final displacement after gel contact for deep insertion was about 1 mm.

FIGS. 24A-24B display an in vivo setup of a rat brain (FIG. 24A) and a monkey brain (FIG. 24B) recording. Scale bars: main figures: 1.5 cm, inset: 1.5 mm.

FIG. 25 displays different frequency bands of recorded signals from the rat brain.

FIG. 26 displays a percentage of channels that record single units (left bar) and visually evoked single units (right bar), showing a consistently high-yield single-unit recording by ROSE probes across animals (N=5).

FIG. 27 displays the number of single units recorded in each subcortical region of rat visual cortex after the onset of visual stimulation.

FIG. 28 displays spike amplitudes and noise floor recorded across 5 weeks (N=5), showing a stable recording across 5 weeks, 5 animals. Data: mean±MSE.

FIG. 29 displays a histology image of tissue-device interface from a 16-shank ROSE probe 1 week post implantation.

FIG. 30A displays representative histology images of the tissue-device interface from ROSE probe and traditional planar Si probe 1 week post-implantation. The neuronal marker (NeuN) shows that the neuron density around the probe hole is comparable between flexible ROSE and stiff Si. The microglia (Iba-1) marker shows less microglia activation around ROSE probe hole than the stiff Si probe hole. Scale bar: 100 μm. The asterisk marks the center of the probe hole.

FIG. 30B displays quantitative tissue fluorescent intensity analysis of one-week post-implantation samples. 10 μm wide concentric bins that centered on the probe hole are created, and tissue markers are analyzed within each bin and plotted as a function of distance to the hole (N=9 images per group). Manually counted neuron density in ROSE group is overall statistically similar to the Si group (p=0.3121). NormalizedIba-1 intensity shows that ROSE probe has overall significantly less microglia activation, especially within 0-50 μm region from the probe hole (p<0.0001). “ns”=not significant, ****p<0.0001, Two-way ANOVA with Sidak's multiple comparison. Data is shown as the mean and standard error of the mean (MSE).

FIG. 31 displays different frequency bands of recorded signals from the monkey brain.

FIGS. 32A-32B display evoked LFP recordings from a monkey brain under 2 trials of visual stimuli.

FIG. 33A displays mapping of electrode channels that recorded MUA from the monkey brain. Z axis is the electrode number from 1 to 8 (H to A).

FIG. 33B displays insertion depth dependence of MU yield recorded by the 256-ch ROSE probe.

FIG. 34A displays a simulation setup which includes a top board, a cushion layer, and a PCB layer with 32×32 BGA.

FIG. 34B displays the maximum principal strain distribution on one quarter of the 32×32 ROSE I/O pads.

FIG. 35A displays predictions of positions of probes on a 3D ROSE probe with a top view illustration of the 3D ROSE probes; d is the thickness of PDMS spacer and MEA, which controls the distance between loops, OA is the distance between the first shank and the center of the micro-mandrel radius. Point B is an arbitrary shank location.

FIG. 35B displays a side view illustration of the 3D ROSE probe structure and its geometric picture. Point C is an assumed electrode location in the shank.

FIG. 36 displays a 4-laminate structure for each layer of the ROSE probe.

FIG. 37 displays insertion footprints detection, enabled by microscopic images of brain slices and a custom python script. Scale bar: 500 μm.

FIG. 38A displays insertion footprints of a 256-shank ROSE probe at 1200 μm below brain surface. The ROSE probe was inserted 2 mm into the ex vivo pig brain. Scale bar: 400 μm (i), 200 μm (ii), 100 μm (iii), 25 μm (iv). The dashed box represents the original device's cross-sectional footprint

FIG. 38B displays insertion foot print area measured from ROSE probes with various shank widths (30, 40, and 55 μm). The brain slice used for measurement is 1200 μm below the brain surface, with 131 footprints measured for each shank width.

FIG. 38C displays the measured width, thickness, area, and perimeter of the insertion footprints (N=131 for each shank width). Data shown: mean±SD.

FIG. 39A displays the location of the inserted ROSE probe inside rat brain. The 3D brain model was illustrated with shadings representing different brain regions. Regions related to the visual cortex are labeled.

FIG. 39B displays spatial-temporal spike firing rate mapping shown in a rat brain model. Brain regions in visual cortex are labeled with shadings. The visual stimuli started at t=0 ms.

FIG. 39C displays multi-regional mapping of spike firing rate using 3D ROSE probe configuration by averaging data collected in 5 trials of visual stimulation (2s in each), showing the transmission of visually evoked responses from V1m to other regions.

FIG. 39D displays the number of spike channels and spike firing rate in different regions of rat visual cortex at different time point after the onset of the visual stimulation. Error bar: standard deviation of data from 5 stimulation trials (2s each).

FIG. 40A displays microscope images in the left, center, and right areas of the planar MEA, showing the misalignment issue of SU-8 encapsulation that happened in the fabrication. The shrinking of the substrate happened in the procedures with high temperatures (such as ion milling and Ni deposition).

FIG. 40B displays microscope images of the shanks after solving the misalignment issue by baking Kapton substrate at 250° C. for 1 hour before the fabrication.

FIG. 41 displays a PCA analysis of a single channel. Inset: spike waveforms from 2 different clusters. Scale bar: 50 μV (vertical), 1 ms (horizontal).

FIG. 42 displays Table 1, which shows detailed parameters of the 256-ch 3D ROSE probe with different shank numbers, shank pitches, and PDMS thicknesses.

FIG. 43 displays Table 2, which shows the maximum strain of the ROSE probe under bending and twisting.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure. Accordingly, the scope of the disclosure is defined only by reference to the appended claims.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

Embodiments of the present disclosure includes a highly scalable, rolling of soft electronics (ROSE) method to achieve a monolithic 3D neural ROSE probe of varying dimensions. Unlike stacking or other known assembly techniques, the ROSE method directly and rapidly transforms a planar probe into a 3D ROSE probe by leveraging the bendability of flexible electrodes and existing semiconductor fabrication technologies.

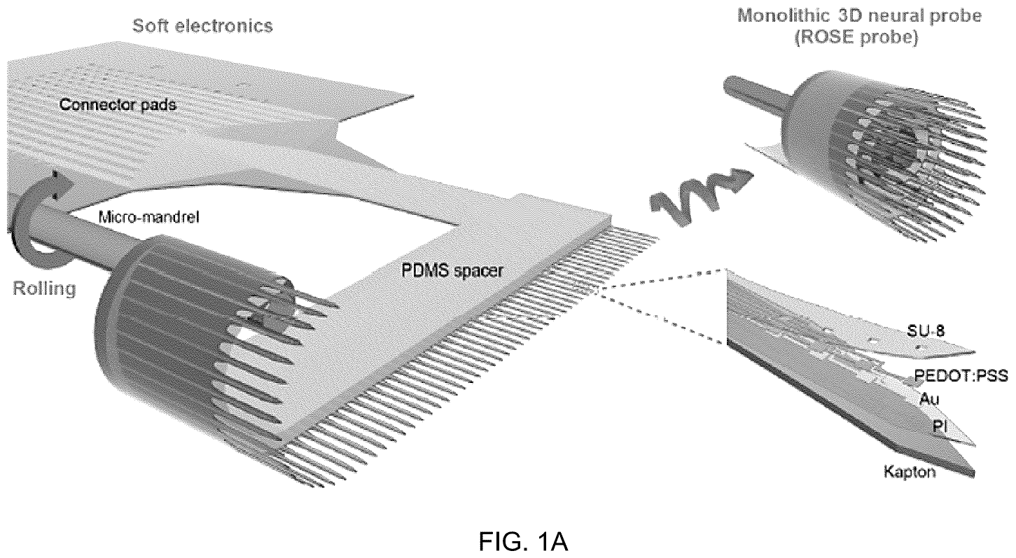

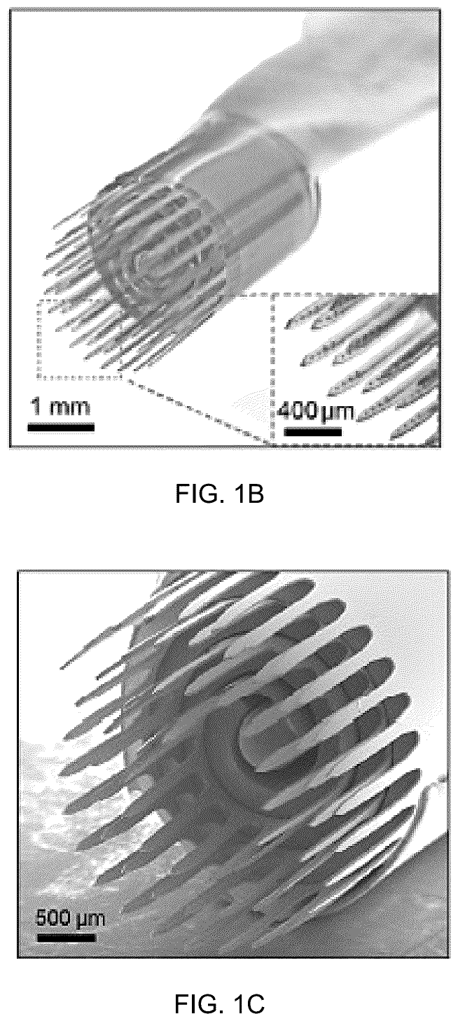

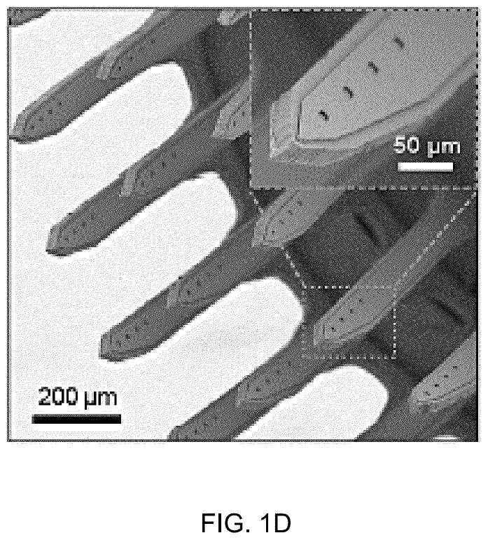

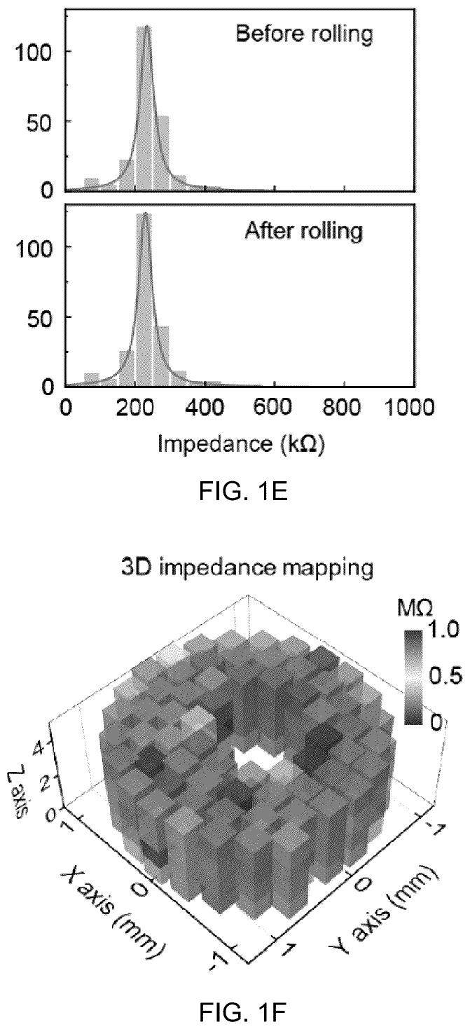

As illustrated in FIG. 1A, embodiments of the present disclosure may include a fully-microfabricated planar component that includes shanks in a row with a desired number of electrodes positioned on each shank. The shanks may be separated by a chosen pitch. The planar component may be rolled by embodiments of the ROSE method to form a ROSE probe.

To facilitate rolling, as described herein and as shown in FIG. 1A, all interconnects may be routed and grouped at one end of the shank row and eventually form a connector pad matrix (e.g., 16×16 for 256-ch probes) on the same planar component (e.g., the same substrate of the planar component). Embodiments of rolling leverages a 50-500 μm diameter micro-mandrel to stick to the far end of the shank row and roll towards the interconnect end. For example, a 500 μm diameter micro-mandrel may be used.

As shown in FIG. 1A, to control the radial electrode spacing among the rolled loops (or spirals) of the ROSE probe, in embodiments, a spacer layer formed by a biocompatible elastomer, such as a Polydimethylsiloxane (PDMS) layer may be bonded on the planar component to the base region connecting the electrode shanks to serve as a soft spacer. As shown in FIG. 1A, the spacer layer may act as a spacer between the rows of the shanks. In some embodiments, the spacer layer may fill all of the space in between each row of shanks. In other embodiments, the spacer layer may fill only a portion of the space in between each row of shanks. The spacer layer may also be formed by elastomers or gels and may have a thickness ranging from 100 to 1,000 μm.

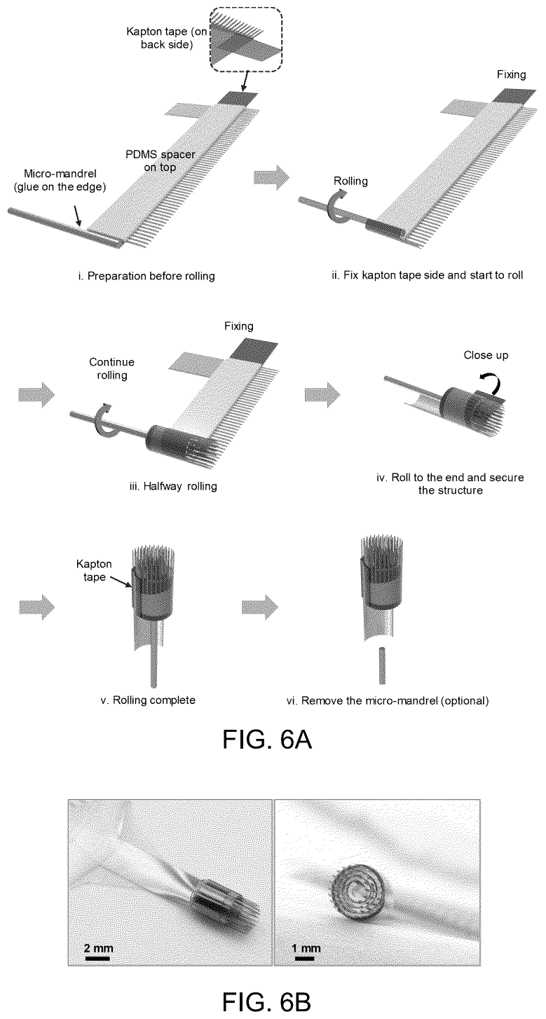

The planar component eventually rolls into a monolithic 3D neural probe (ROSE probe) with a cylindrical form factor where all shanks evenly distribute along a spiral (FIGS. 1B-1D). To retain the circular nature of the ROSE probe, the planar component may include an adhesive portion that may connect the ends of the planar component together to secure the ROSE probe. The adhesive portion may include any medical adhesives/tapes, adhesives, or epoxy. The detailed rolling process of the ROSE method disclosed herein is described in the Examples and in FIGS. 6A-6B.

In embodiments, the planar component may include a substrate layer, an electrode and corresponding interconnects layer, and an encapsulation layer. The planar component may include one or more layers, such as 5 layers shown in FIG. 1A. As shown, Kapton/polyimide (PI) may be used as the substrate layer, Au/PEDOT: PSS may be used as the electrode and interconnect layer, and SU-8 layer may be used as the encapsulation layer. Embodiments of the present disclosure include variations of layers, number of layers, and materials used in each later. For example, the planar component may be Parylene based, liquid crystal polymer based, or SU-8 based. Embodiments of the planar component may have a thickness of 1-100 μm. FIG. 1A displays a flexible Kapton based planar component with a thickness of approximately 25 μm, which includes all the shanks in a row. The planar component may be made of any flexible material that may be rolled into a ROSE probe.

In an embodiment of the present disclosure, 10-1000 shanks may be included in the planar component of the ROSE probe. In some embodiments, the shanks may be evenly distributed along the planar component. In other embodiments, the shanks may not be evenly distributed. Embodiments disclosed herein include all variations of planar positioning of the shanks.

In embodiments, the shanks may be pointed to allow for penetration. For example, the shanks may have a tapered profile with a tip angle of 20°. Other angles are possible and may be dependent on penetration.

Embodiments of the present disclosure may include shanks of varying lengths. In embodiments of the ROSE probe, all of the shanks may be of the same size or dimensions, or the shanks may have varying sizes and dimensions. The length of the shanks may depend on penetration. For example, longer shanks may be used to penetrate the shanks of the ROSE probe deeper into tissue, such as brain tissue. In other embodiments, the shanks of the ROSE probe may not have to penetrate as deep into the tissue, and shorter shanks may be used. The shanks may have a length from 0.5-5 mm, a width from 10-1,000 μm at the widest point, and a thickness from 2-100 μm.

In an embodiment of the present disclosure, 1-256 electrodes may be included in the ROSE probe. The electrodes may be disposed on one or more shanks. Each shank may include the same number of electrodes or a different number of electrodes. In an embodiment, the electrodes may be positioned in any location on the shank. For example, the electrodes may be evenly spaced on the shank from the tip of the shank to the base of the shank. In embodiments, the electrodes may be separated by a pitch from 20-300 μm.

Embodiments of the ROSE probe include a connector pad matrix, which includes the interconnection of each electrode of the ROSE probe. In embodiments, a 16×16 connector pad matrix may be used with 256-channel electrodes. In other embodiments, a 32×32 connector pad matrix may be used with 1024-channel electrodes. For example, in embodiments of the 16×16 connector pad matrix, 64 shanks may be used with 4 electrodes included in each shank. Different variations are possible and disclosed herein.

The following examples are presented to illustrate the present disclosure. They are not intended to be limiting in any matter.

### Example 1

The following example provides a description of embodiments of a monolithic three-dimensional neural probe from deterministic rolling of soft electronics and a description of in vivo validation.

Cognition and behavior both rely on coordinated activity from neural circuits distributed in three dimensions. However, to date, the mainstay neural probes are constrained to 2-dimensional interfacing with the brain, largely due to the predominant planar semiconductor fabrication process.

This example has demonstrated deterministic ROSE from versatile 45 planar designs and systematically studied the insertion dynamics of ROSE probes containing hundreds of electrodes. In addition to demonstrating compelling single-unit spike recording yields in vivo in both rodent and non-human primate models, Embodiments of the ROSE probe also revealed microscopy-like 3D spatiotemporal mapping of spike activities in the rodent visual cortex, with demonstrations of over-month-long recording stability and promising 3D decoding performance of visual orientation. Embodiments of the present disclosure offers general applicability in soft bioelectronics and provides a pragmatic pathway to establish 3D neural probes.

This example demonstrated deterministic rolling from a wide range of planar designs at the 256 channel (ch) level, resulting in 3D probes with tunable probe geometries. Systematic insertion studies coupled with mechanical modeling revealed that avoiding slipping-induced instability can help achieve successful insertion in addition to preventing conventional buckling. Embodiments of the present disclosure further validated the ROSE probe in vivo in rodent and non-human primate (NHP, rhesus monkey) models. Neuronal recordings were performed from ROSE probes with hundreds of electrodes and demonstrated compelling yields of SU spike recording and clear temporal responses across far distant areas of the visual cortex. Immunohistological studies in rodent models showed that ROSE probes reduce tissue stress and inflammatory reactions compared to traditional stiff Si probes, which corroborated their stable semi-chronic recording performance. Additionally, recording using ROSE probes in awake rodent models revealed clear 3D-distributed orientation tuning and demonstrated higher decoding performance using the 3D-recorded units compared to those from any single electrode plane. Embodiments of the ROSE probe described herein address an unmet need in known neural interfaces and also establish embodiments of the ROSE method and its underline design guidelines, which are generally applicable to soft bioelectronics.

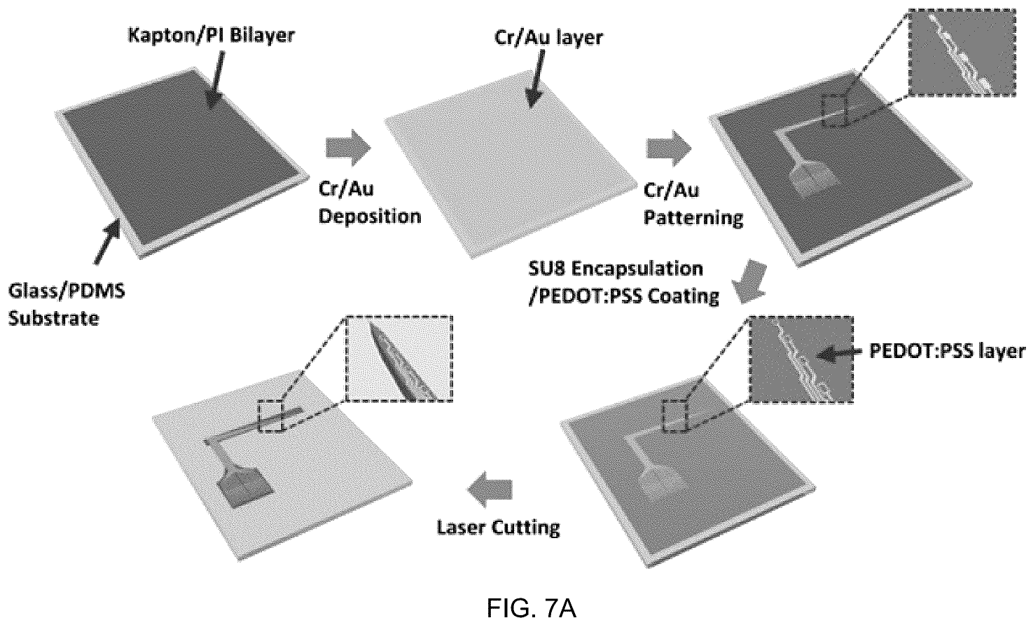

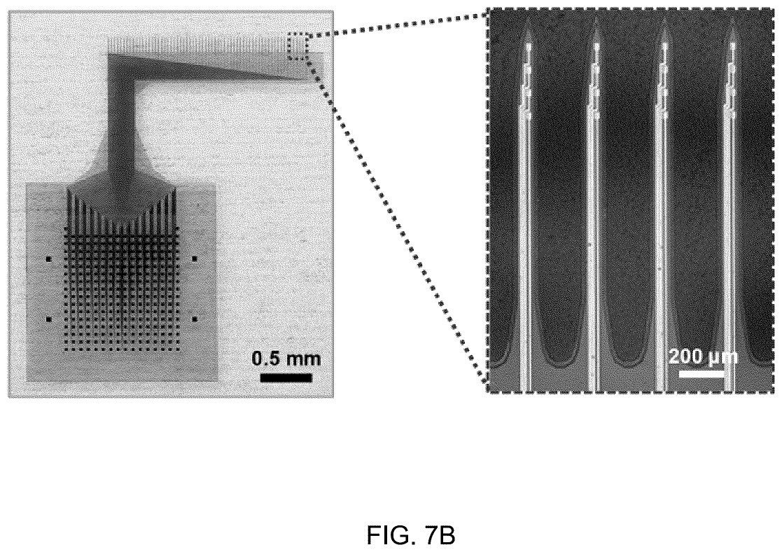

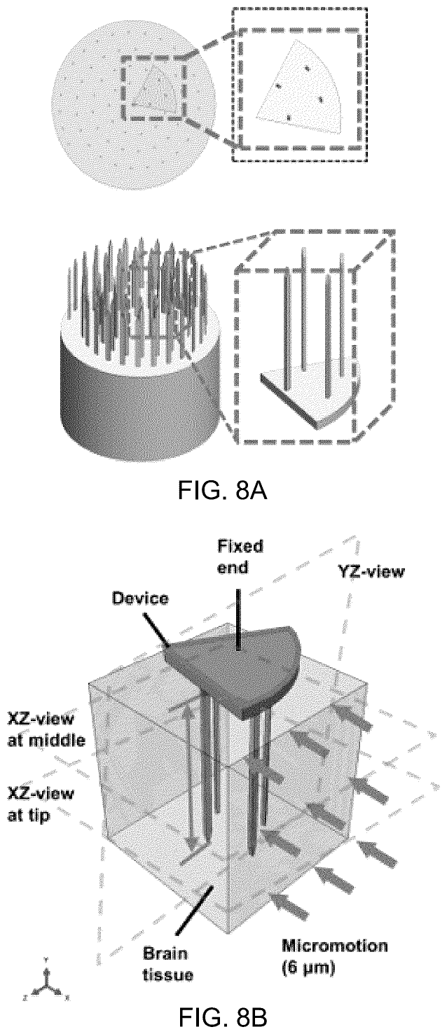

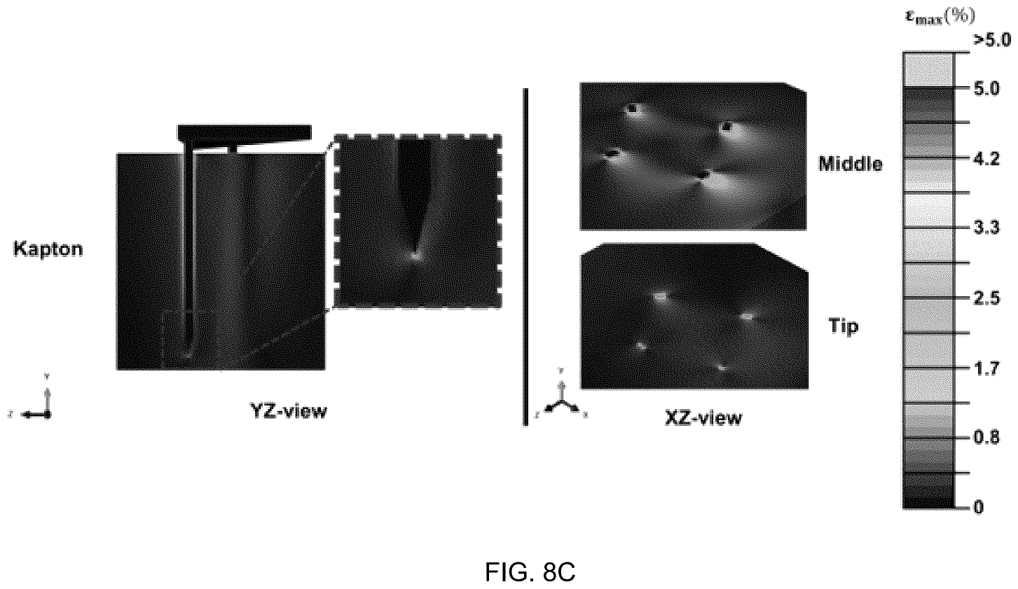

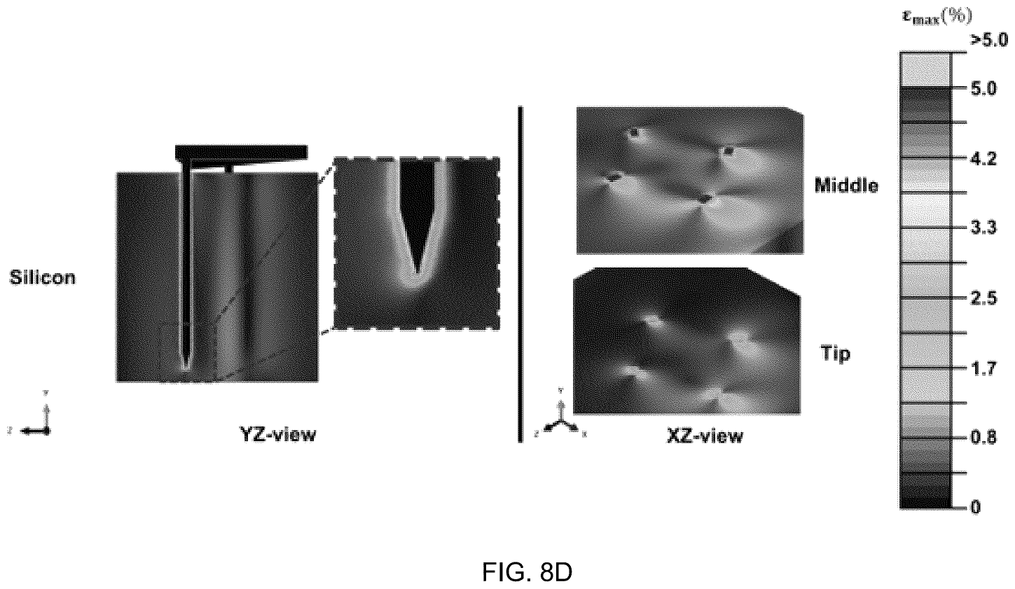

Embodiments of the ROSE probe offers unique scalability advantages because of its monolithic nature as compared to existing 3D probe systems that deploy a stacking approach. Embodiments of the ROSE probe's monolithic and flexible construction provides several distinct benefits. First, embodiments of the ROSE probe allow for a customizable, true 3D arrangement of electrodes, enabling tailored volumetric recordings for different applications. Second, beyond the specific layout of the electrode shanks and interconnects, embodiments of ROSE method leverage thin-film flexible electronics with circuit design and fabrication (FIGS. 7A-7B), which is generally a robust and cost-effective manufacturing technique. Third, the use of flexible materials like polyimide, with a Young's modulus two orders of magnitude lower than Si and tungsten, allows for mitigating the mechanical mismatch between implanted probes and surrounding brain tissue. For example, a finite-element analysis (FEA) simulation shows significantly lower strains induced by brain micromotion in shanks made of polyimide compared to those made of Si (FIGS. 8A-8D), an important factor known to reduce immune responses.

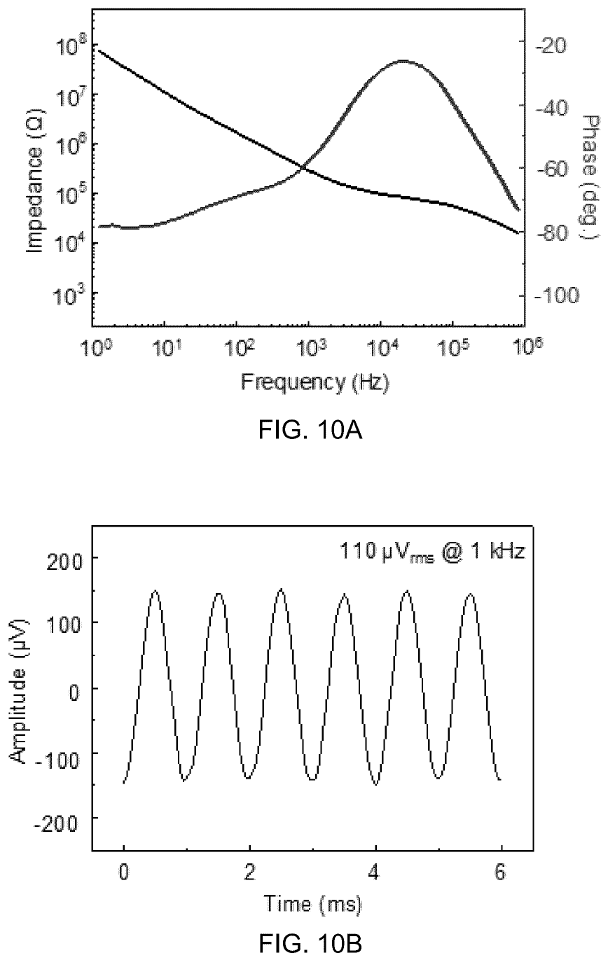

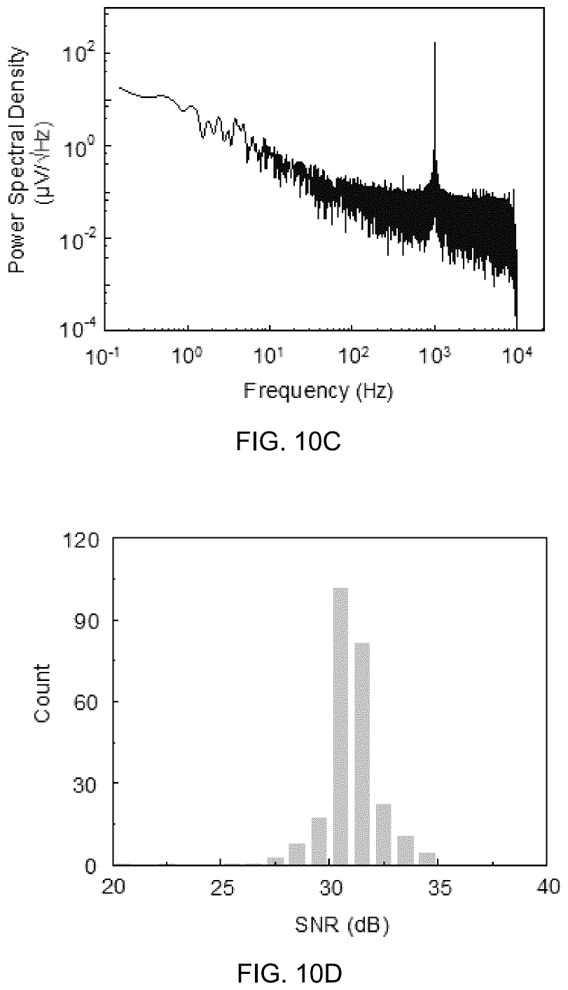

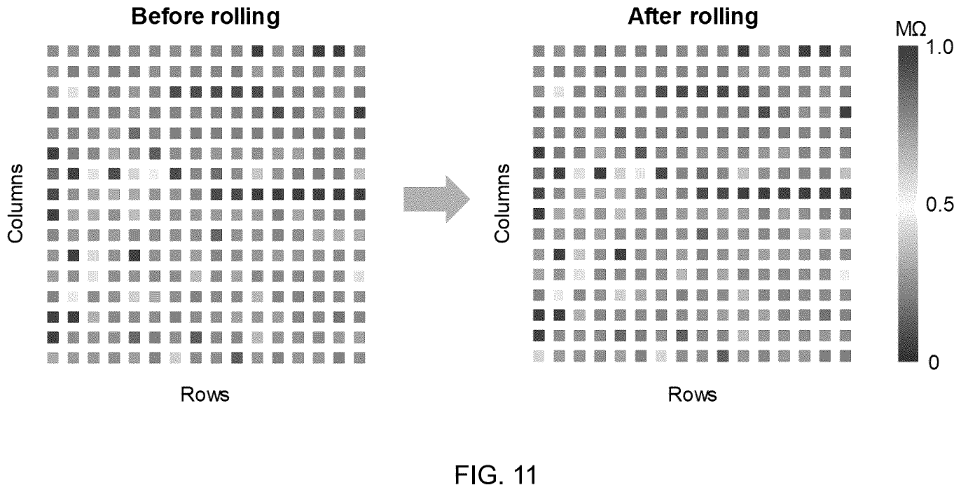

Due to the lack of compact and reusable connectors for large-scale soft probes, embodiments of the present disclosure may include a printed-circuit-board (PCB)-based connector, MagMatrix, to connect ROSE probes and rigid data acquisition electronics (FIGS. 1E, 9A-9D, and 11). Details of the MagMatrix connector are described in Example 4. Impedance characterization has revealed negligible difference before and after rolling (FIGS. 1F, 10A-10D, and 11) for embodiments of the 64-shank, 256-channel (256-ch) probes with an average impedance of 239.1±70.2 kΩ at 1 kHz and with a yield of 92.2%, suggesting that embodiments of the ROSE probe possess the same performance as its planar device precursor. The impedance of functional channels in a ROSE probe has also shown uniformity (FIG. 1F).

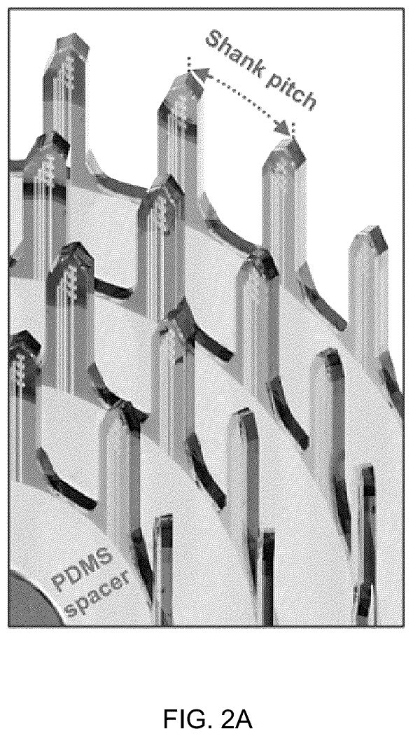

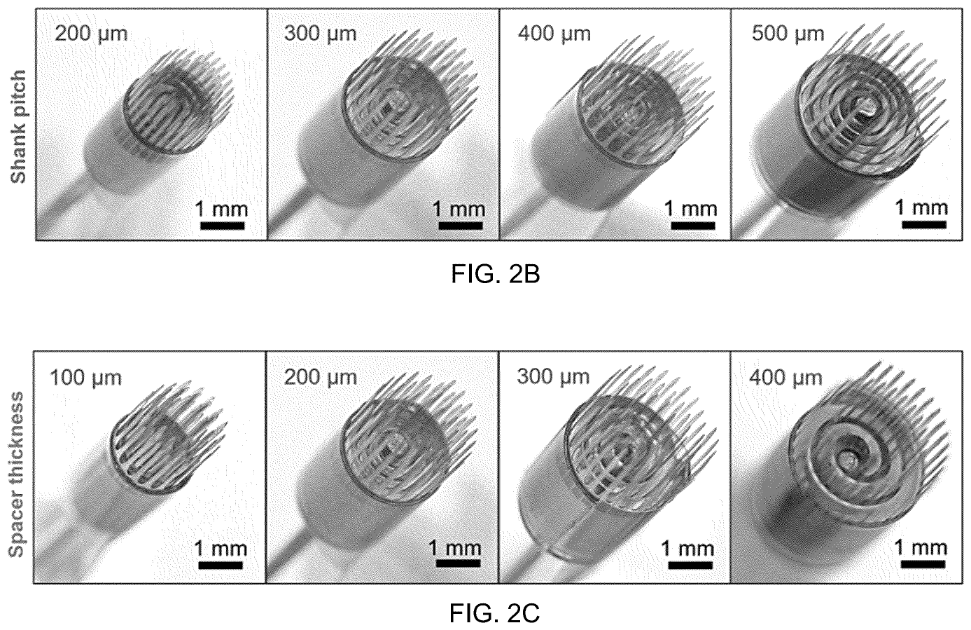

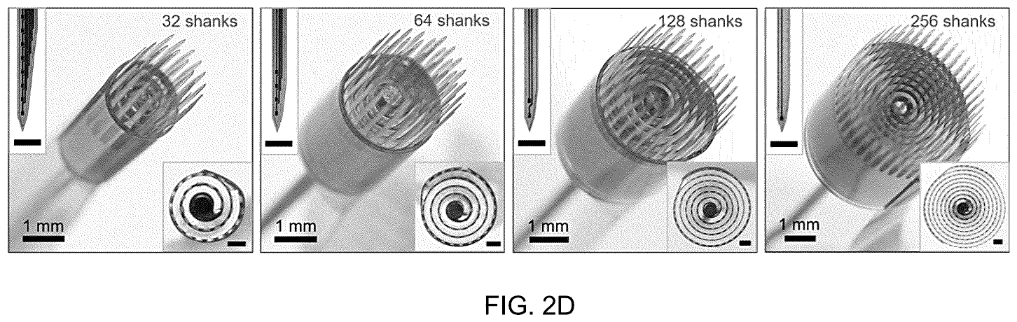

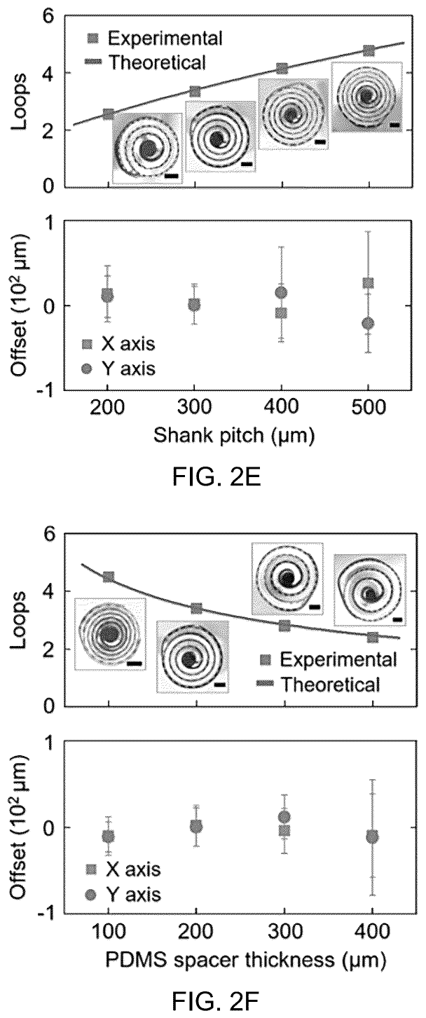

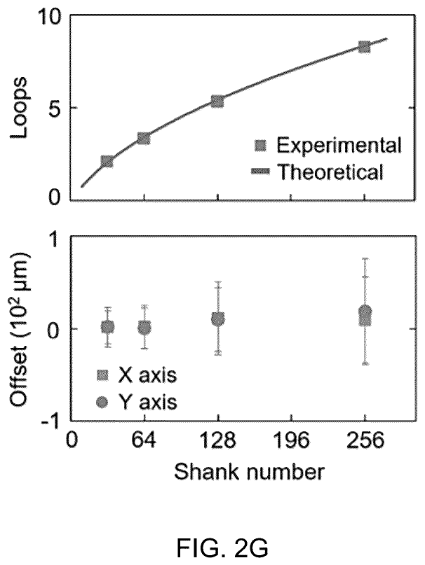

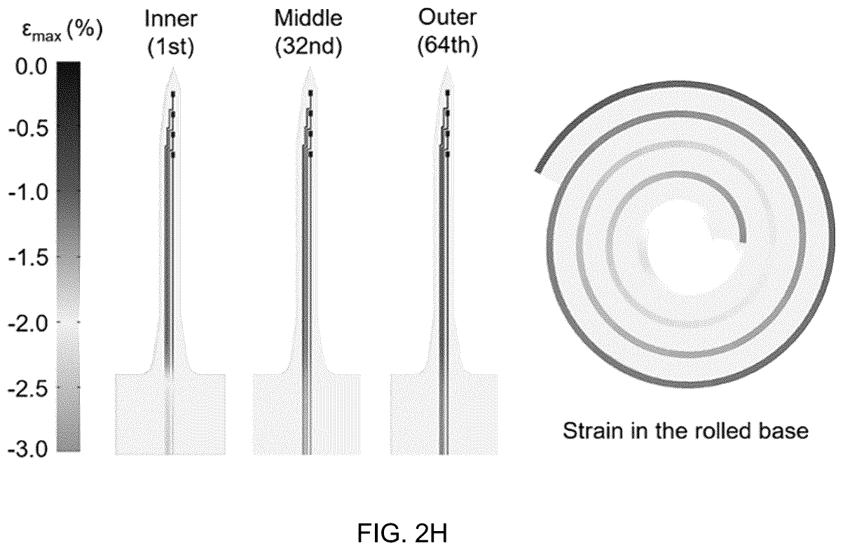

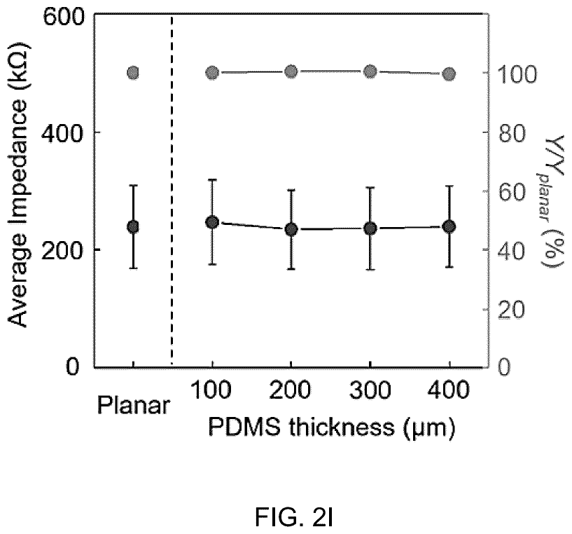

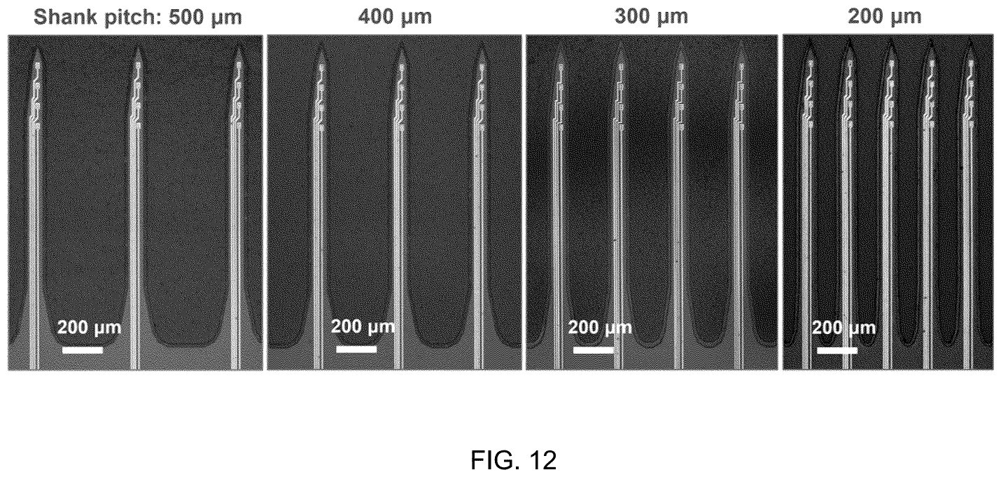

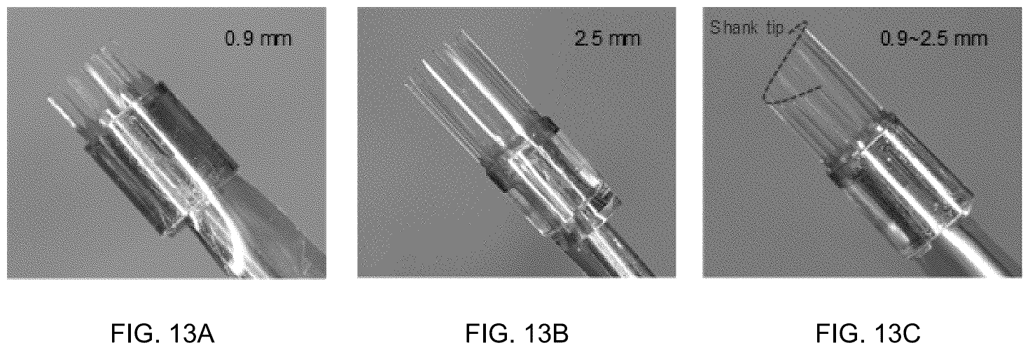

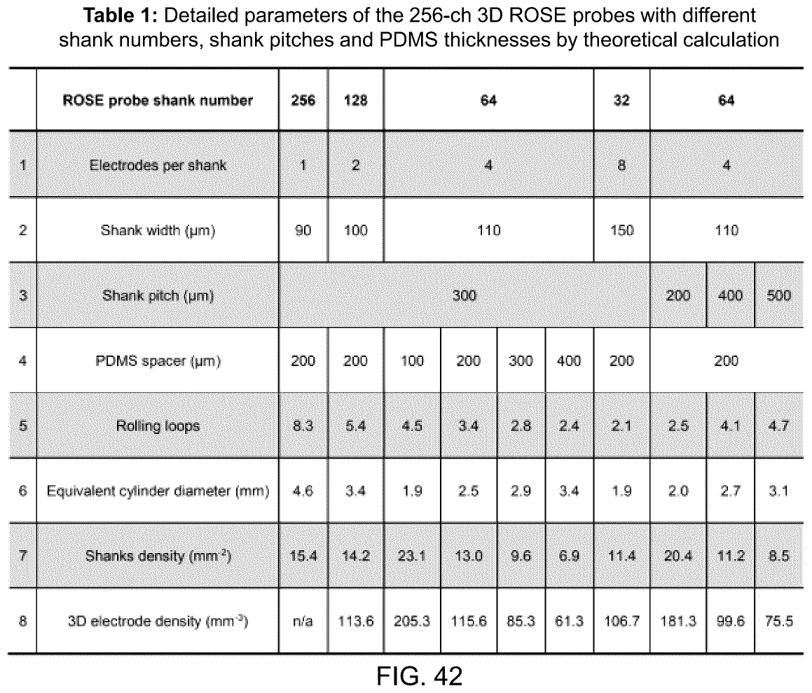

Embodiments disclosed herein may be applicable to a wide variety of planar designs of flexible probes, which through rolling, generate 3D soft probes with precisely controlled dimensions and electrode configurations in three dimensions. Embodiments of the present disclosure highlight the robustness of this methodology for highly customizable designs based on experimental and anatomical demands. Embodiments of the ROSE probes with different sizes and densities may be achieved by varying probe parameters, including the number of shanks, shank pitches, and the PDMS spacer thickness, all at the 256-ch level (FIGS. 2A-2D, 12, and Table 1 shown in FIG. 42). Moreover, embodiments of the design can be further extended by varying the length of individual shanks, forming customizable 3D geometries to serve specific recording needs (FIGS. 13A-13C).

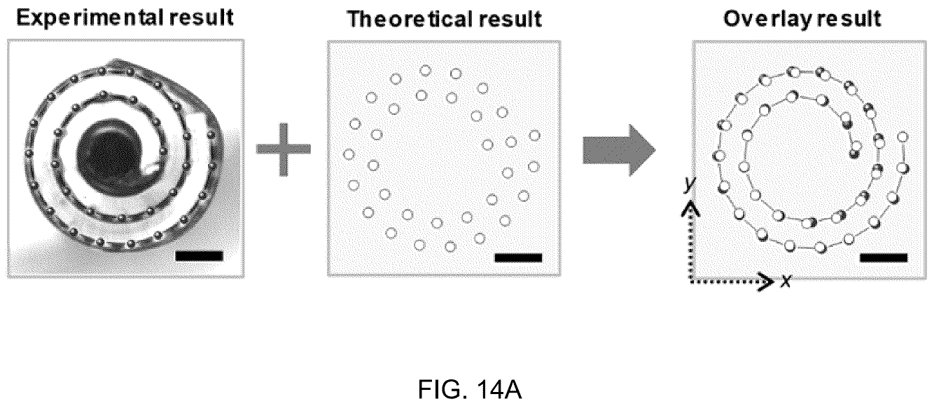

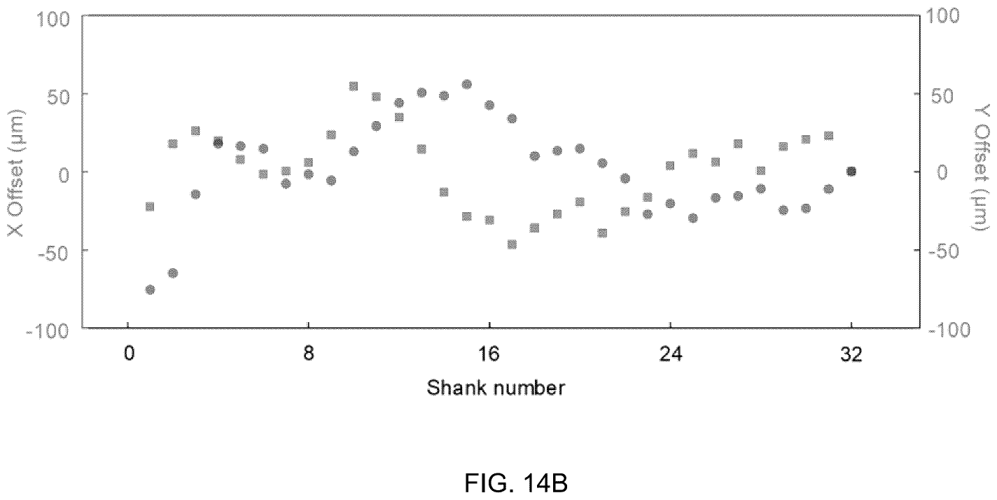

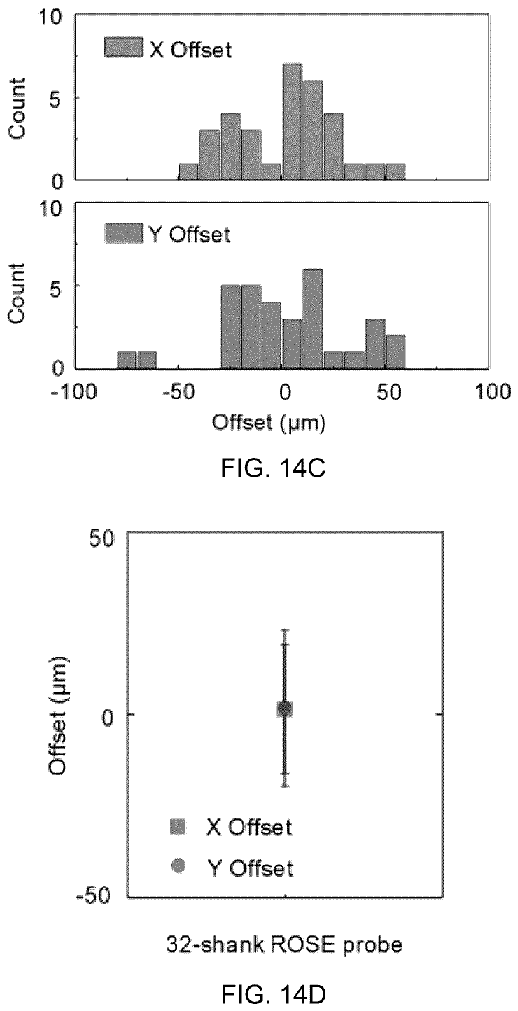

As shown in FIGS. 2E-2G, embodiments of the rolling process of the ROSE method are highly deterministic, yielding a predictable number of loops (or spirals) and shank positions. Based on the theoretical prediction by an “Archimedean spiral” model, the top view structure of the ROSE probe was calculated (Example 3). By knowing the 2D design of shanks, the initial rolling radius, the PDMS spacer thickness, and the coordinates of each specific shank in the spiral structure (hence all electrode positions) can be determined and designed before rolling. The exact method to attain the offsets between experimental and theoretical results are shown in FIGS. 14A-14D.

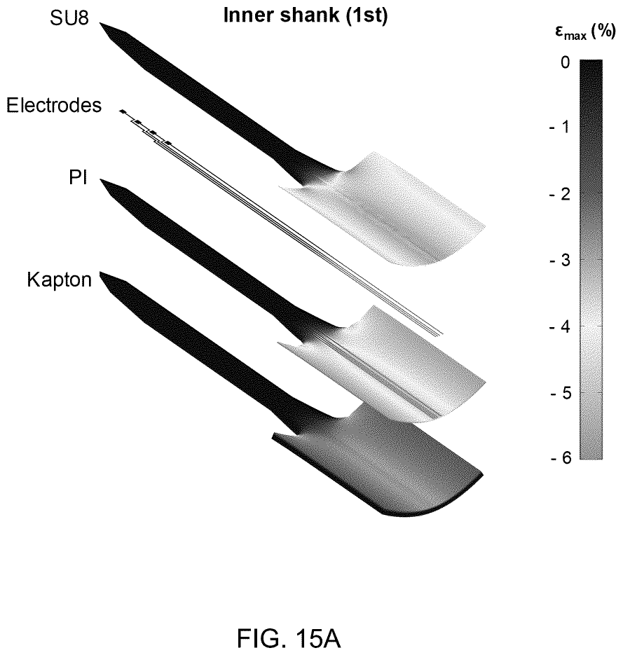

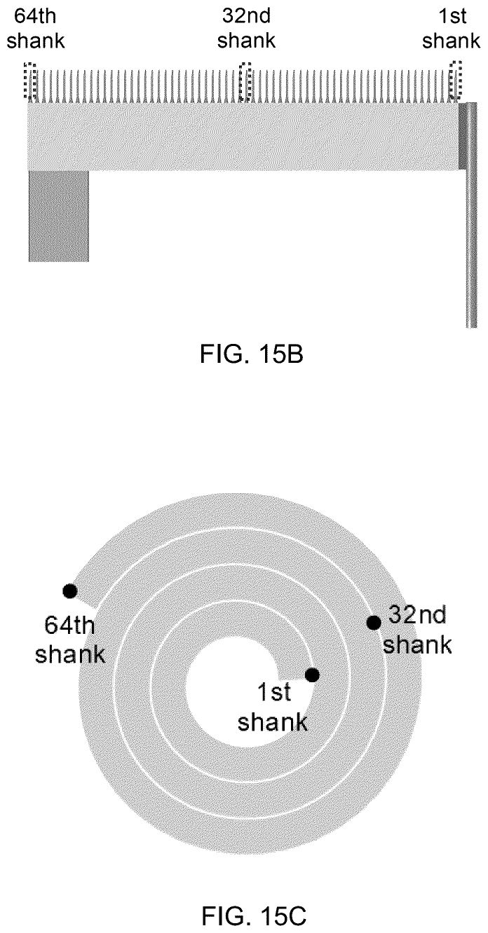

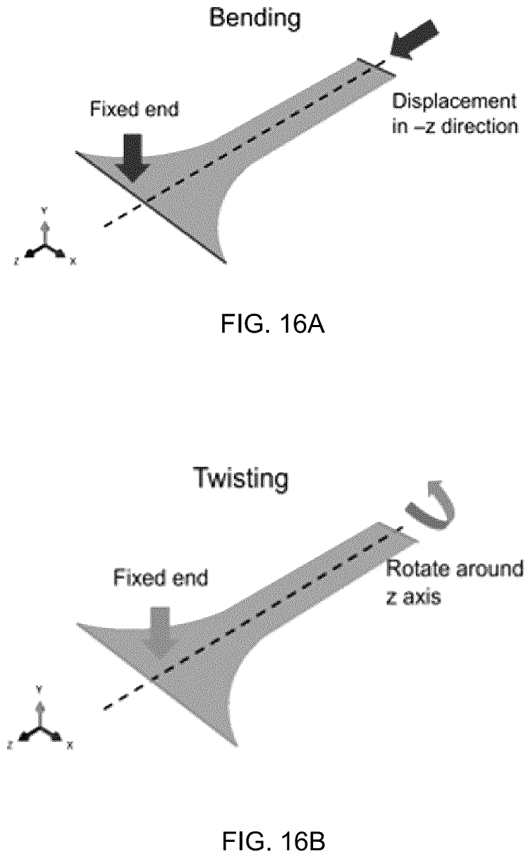

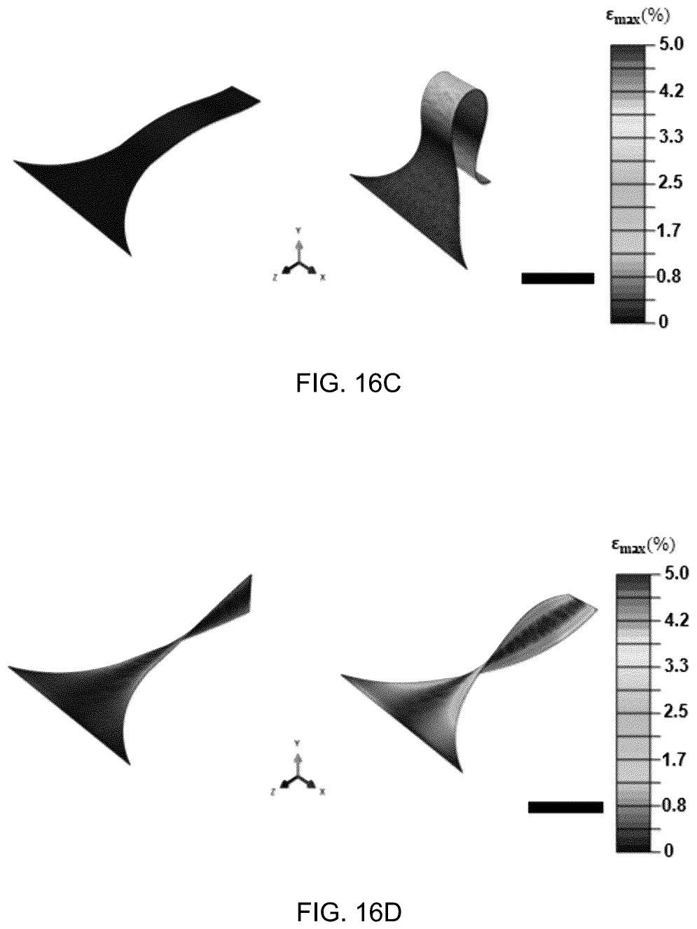

Embodiments of the ROSE method is also reversible and non-destructive. To ensure that small radii of curvature do not negatively influence the recording quality, the strain originated from rolling was further investigated. FEA results revealed that rolling influences only the base region of the shanks. In contrast, the strain of electrodes in the shanks is negligible (FIG. 2H) due to the narrow and long nature of the shanks, which mechanically isolates the electrode region from being severely deformed during rolling. Even in the rolled base region and innermost region of the ROSE probe (with the largest curvature), the highest strain in the electrode interconnects is still less than 3% (compressive, FIGS. 15A-15C), far less than the fracture strain of gold thin films on a flexible substrate. Theoretical modeling also confirms the gradual decrease of strain from the inner to the outer area of the spiral base due to the increasing radius of curvature, in agreement with the FEA results (−2.82%, −1.38%, and −1.03% in theory versus −2.66%, −1.19%, and −0.94% in FEA for the #1, #32, and #64 shank, respectively, in Example 6). Since the rolling operation results in negligible strain, the ROSE method preserves electrode performance, further vindicated by no impedance degradation from the planar device to ROSE probes with various PDMS spacer thicknesses (FIG. 2I). FEA simulation results indicate that the probe ‘neck’ is safe to experience a bending radius of up to 1.12 mm and can be twisted up to 180° (FIGS. 16A-16D and Example 7).

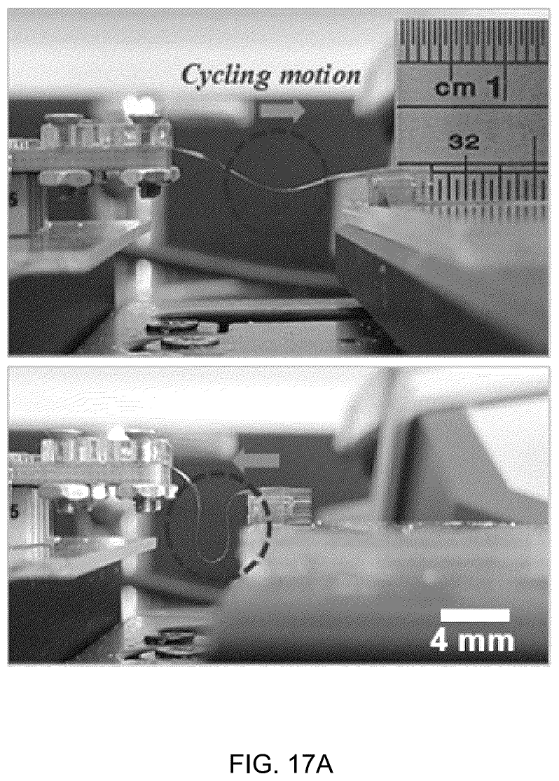

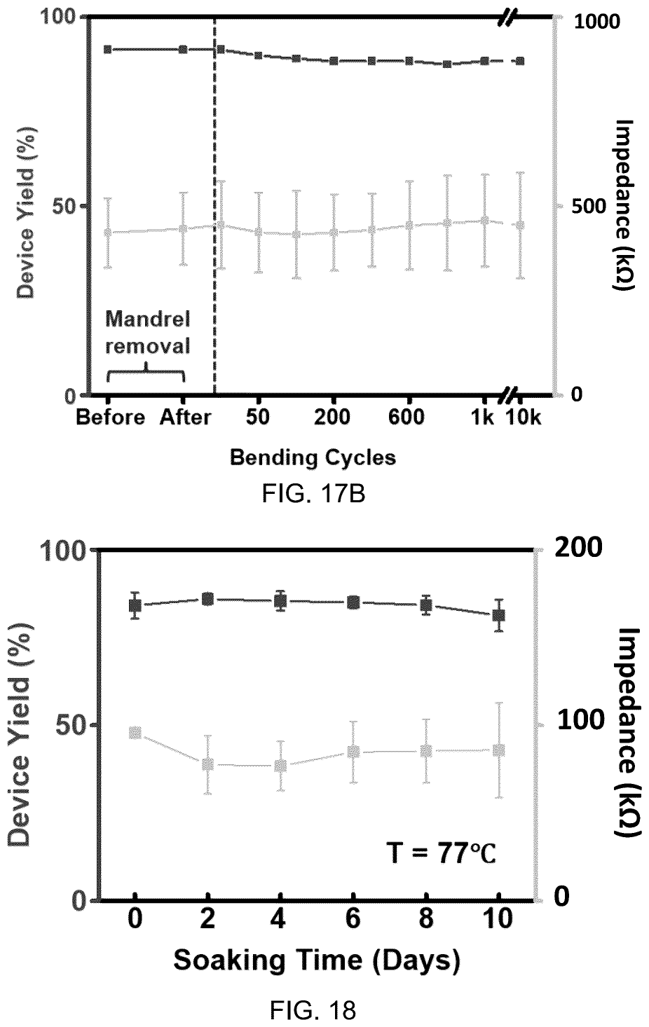

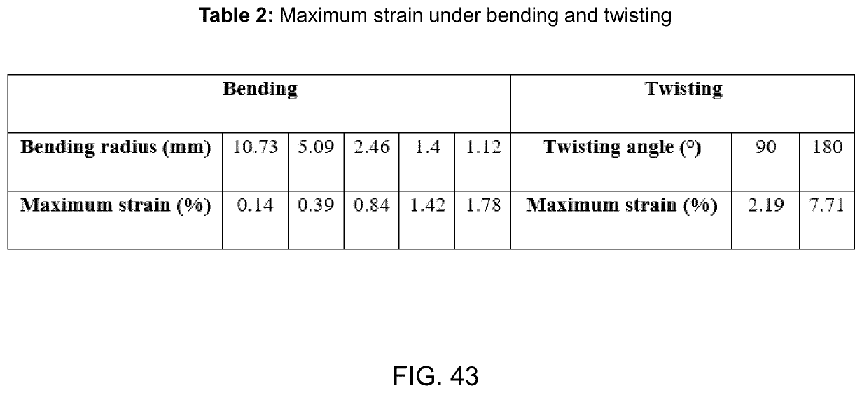

Table 2, shown in FIG. 43, displays the maximum strain in the probe under bending and twisting by FEA modeling. Besides simulation, the reliability of the ROSE probe ‘neck’ was validated with cyclic bending tests up to 10,000 cycles (FIGS. 17A-17B) and demonstrated a consistent probe yield and average impedance value after 10,000 cycles of bending. In addition, after being soaked in a phosphate-buffered saline (PBS) solution at 77° C. for 10 days (equivalent to 160 days at 37° C. according to the accelerated aging calculation method specified in ASTM F1980), the probe's yield dropped less than 10% (N=4, FIG. 18), indicating the strong reliability of the ROSE probe. Maintaining stable electrode impedance is a crucial first step for achieving chronic brain recordings.

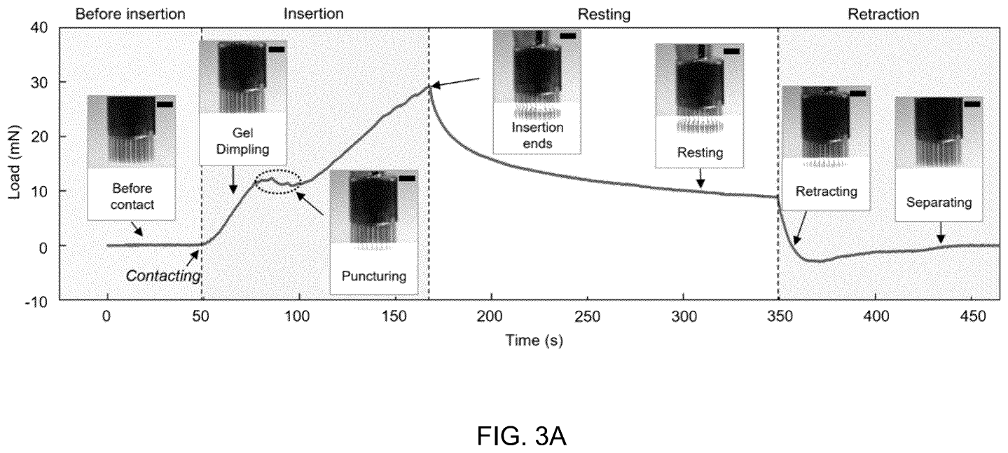

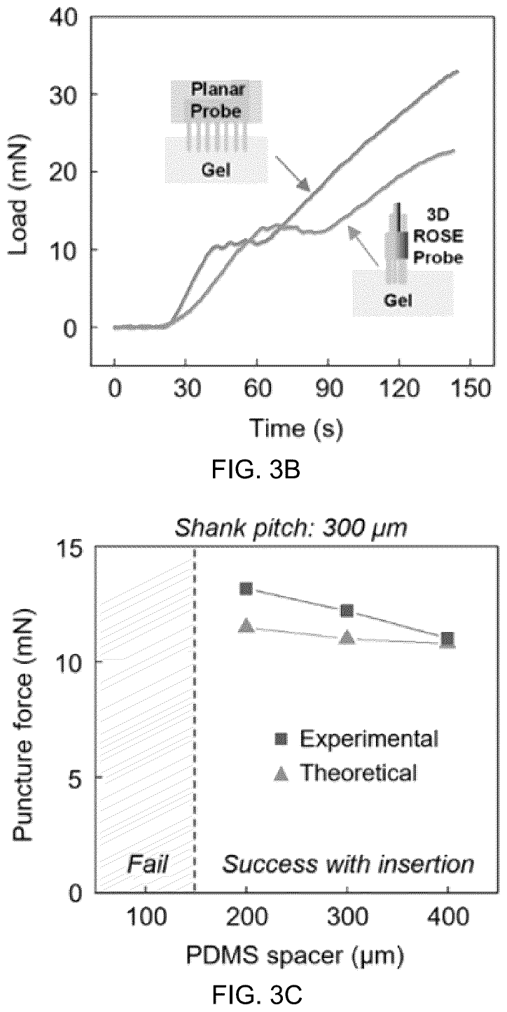

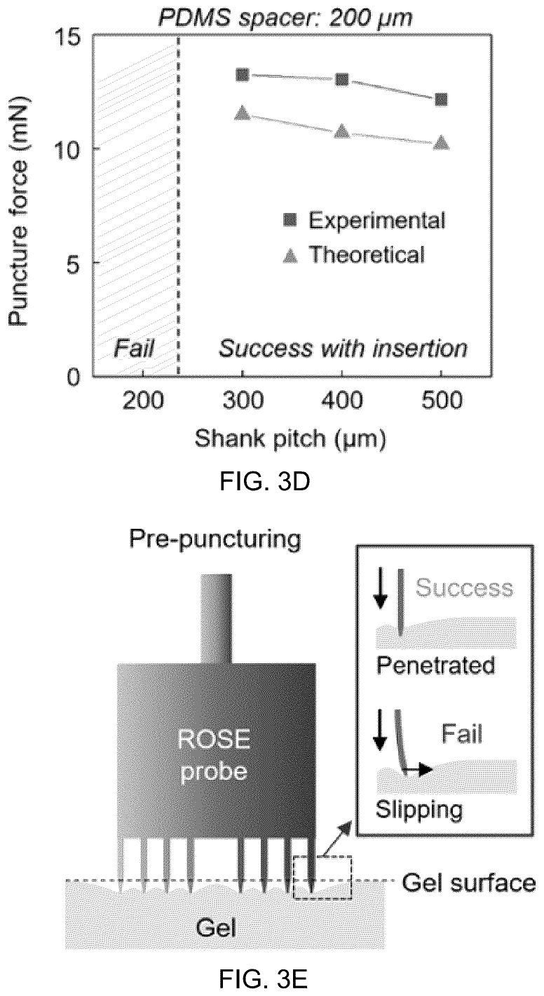

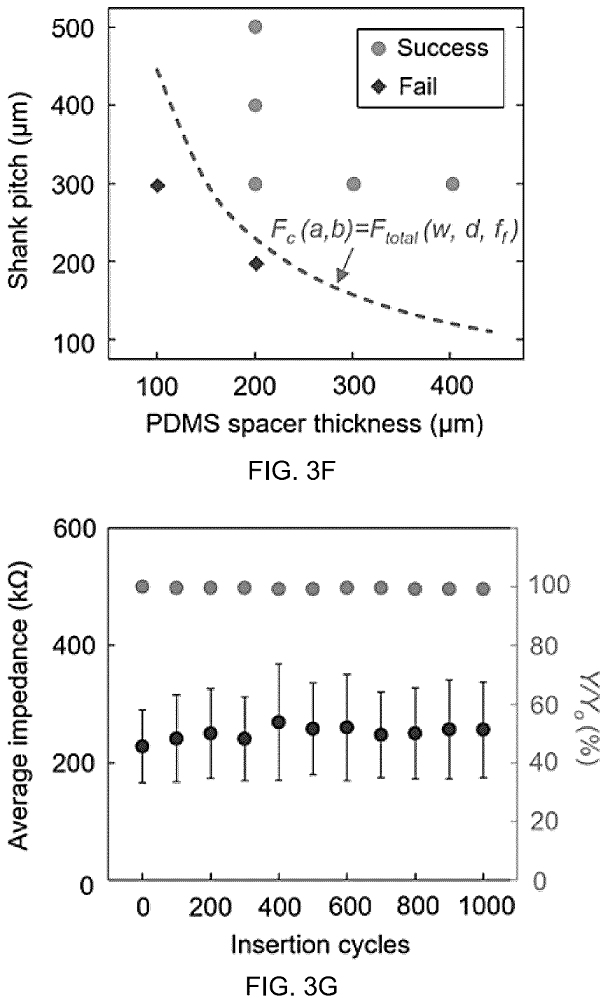

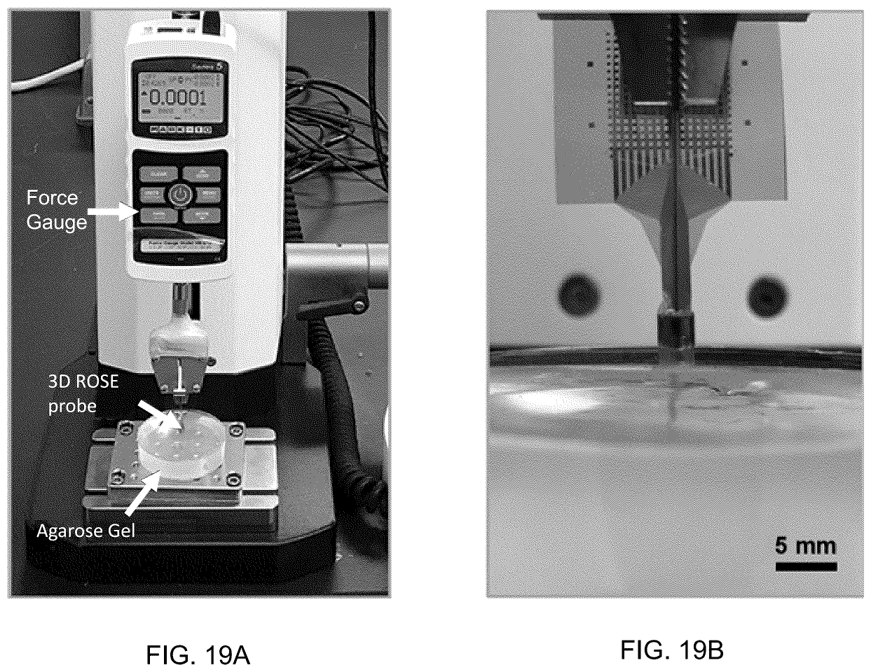

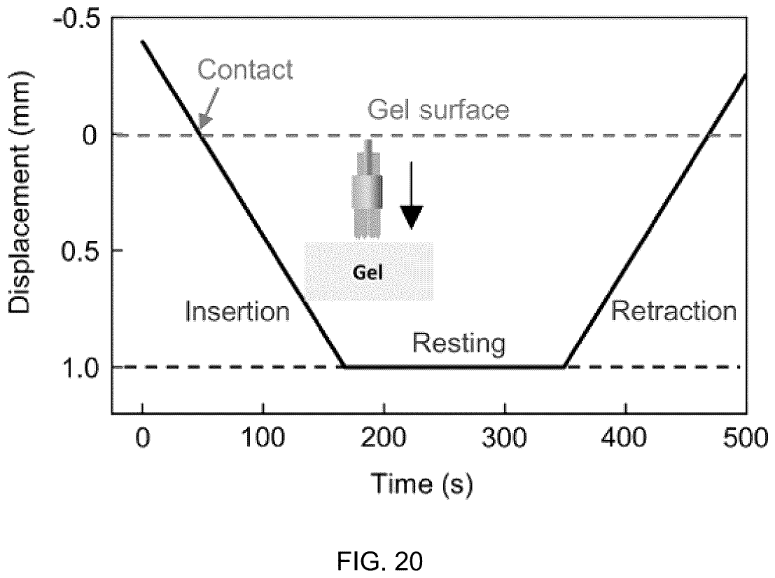

Embodiments of the ROSE probe may be inserted into the brain as a neural probe. To shed light on the implantation process, an embodiment of a 64-shank ROSE probe with varying shank pitch and PDMS spacer was used as a model system and its insertion dynamics was studied with brain phantoms. A representative quantitative force dynamic measurement in 0.6% agarose gel models revealed the whole insertion process with distinctive phases: dimpling, puncturing, inserting, resting, and retracting (FIGS. 3A and 19A-19B). Upon touching the gel surface, the contact force initially increases almost linearly as the vertical probe displacement dimples the gel surface. Once the ROSE probe displacement reaches a certain threshold, the contact force first saturates then enters a ‘force drop’ region, indicating the shank tips start puncturing into the gel. Notably, for ROSE probes with appropriate shank density, each 1.5-mm-long shank has been able to insert into the gel without any insertion aids at an insertion speed of 0.5 mm/min. After all shanks successfully puncture the gel, the contact force increases almost linearly again as shanks insert deeper, presumably resulting from the increase of friction between the gel and the shanks. The high-density shanks in the ROSE probe all successfully penetrate the brain phantom without any visually observed buckling. After stopping the 2-min long insertion at a final displacement of 1 mm, the force exhibits stress relaxation of the hydrogel material during a 3-minute resting phase (FIG. 20) due to the recovery of the deformed gel, dropping approximately by ⅔ at the end of this phase. Upon retracting, the force eventually decreases and crosses over zero before reaching the minimum, then slowly goes back to zero when the shanks withdraw from the gel at the same speed.

When comparing the force dynamics of a ROSE probe and its planar device precursor under the same insertion conditions (FIG. 3B), the ROSE probe exhibits a larger puncture force (defined as the peak of the contact force in the puncturing phase) with a later puncture. The insertion forces may be smaller, suggesting strong effects from the inter-shank coupling.

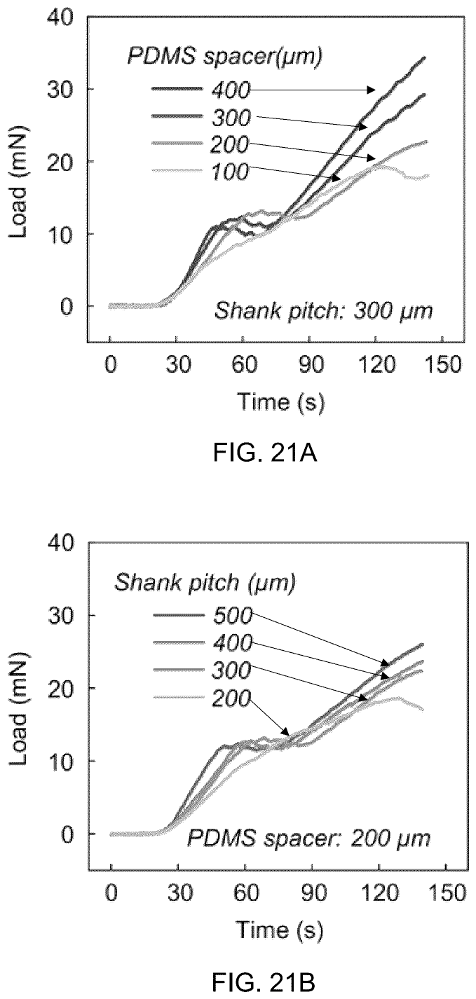

Further, the insertion dynamics were studied, especially for puncture forces from ROSE probes with different PDMS spacer thicknesses (100-400 μm, FIGS. 3C and 21A) and shank pitches (200-500 μm, FIGS. 3D and 21B). Overall, embodiments of ROSE probes with lower shank density (from wider shank pitch or larger PDMS spacer thickness) enter the puncturing phase earlier than embodiments with denser shanks, consistent with the early puncture from the planar device. While the shank pitch does not significantly affect the slope of contact force increase during the inserting phase, larger PDMS spacer thickness increases this slope, eventually leading to larger puncture forces. A theoretical model is developed to estimate the puncture forces, as shown in Example 8. The puncture forces of all inserted cases are in agreement with the theoretical estimations (FIGS. 3C-3D). The most successful embodiments of the ROSE probe corresponded to exceedingly high shank densities.

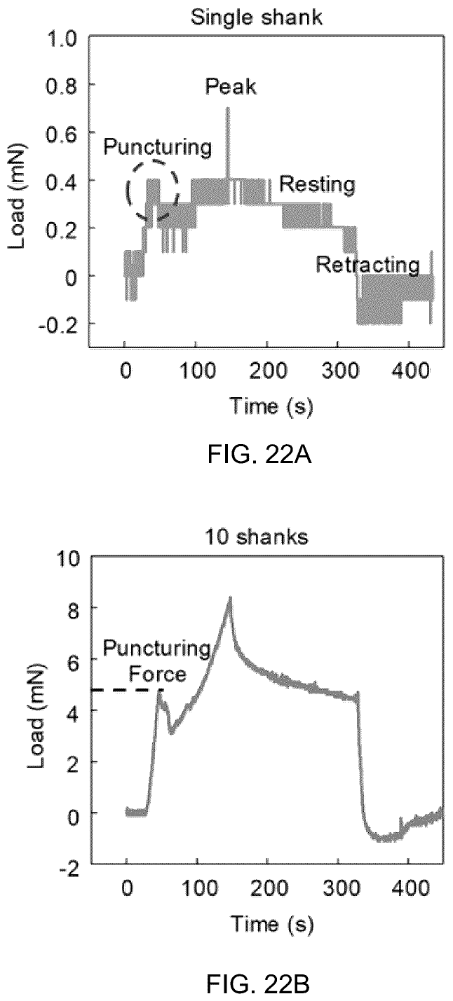

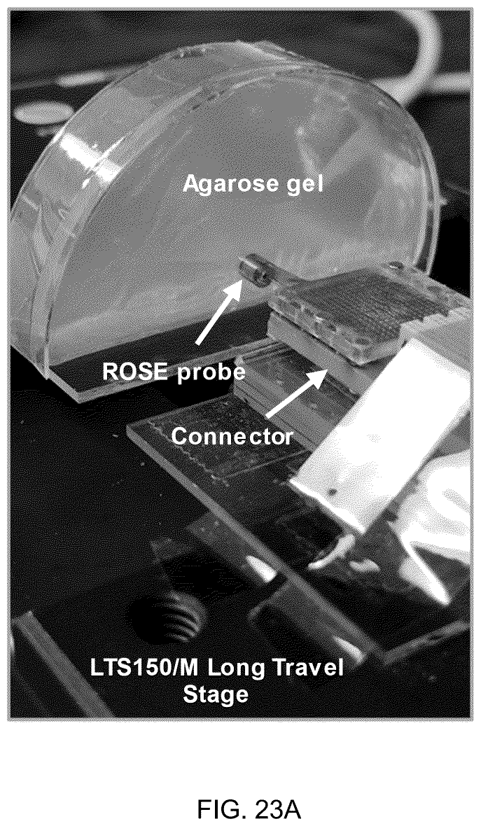

Theoretical calculations indicate that the buckling force threshold of a 1.5-mm-long Kapton shank is approximately 3.58 mN (Example 9), which is about one order of magnitude larger than the puncture force for a single shank, estimated to be Fp˜0.46 mN from the insertion experiments using 10 widely separated shanks (FIGS. 22A-22B). Therefore, conventional buckling is less likely to cause failure in embodiments of the ROSE probe.

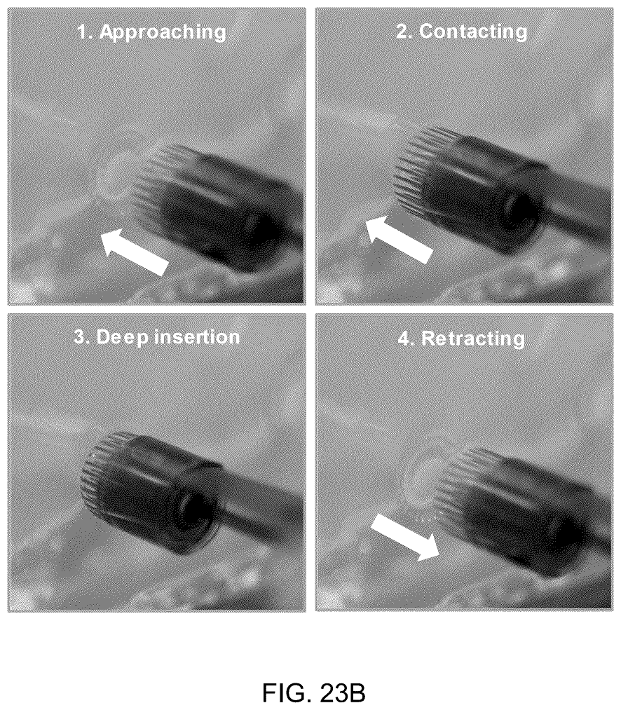

Slipping between the shank tip and the gel surface can happen much more easily when the contact force reaches a threshold, leading to shank deformation and accelerated collapse (FIG. 3E). A theoretical model approximating the ROSE probe shank arrays' contact behavior as a hollow cylinder (Example 8) was developed to predict the critical contact force for shank slipping. The combination of shank pitch and spacer thickness that indicate the onset of slipping before insertion can be analytically obtained by equating the critical contact force of slipping with the puncture force. It is shown in FIG. 3F that this theoretical scaling law (dash line) strongly agrees with experimental observations (circles for successful insertion, diamonds for failed cases). This scaling law of embodiments of the ROSE-probe insertion provides design guidelines for controlling the density of ROSE probes to avoid slipping between the shank tips and the gel with such shank design, thereby avoiding subsequent bending and collapse of the shanks. When designed appropriately under the guidance of this scaling law, ROSE probes are highly robust for insertion. Cyclic brain phantom insertion tests (FIGS. 23A-23B) of a ROSE probe with up to 1,000 insertion cycles led to minimal changes in the average impedance and the electrode yield, indicating the reliability and reusability of embodiments of the ROSE probe during implantation (FIG. 3G).

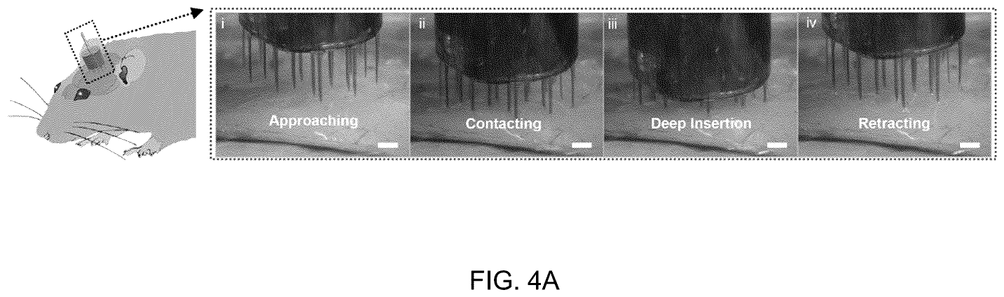

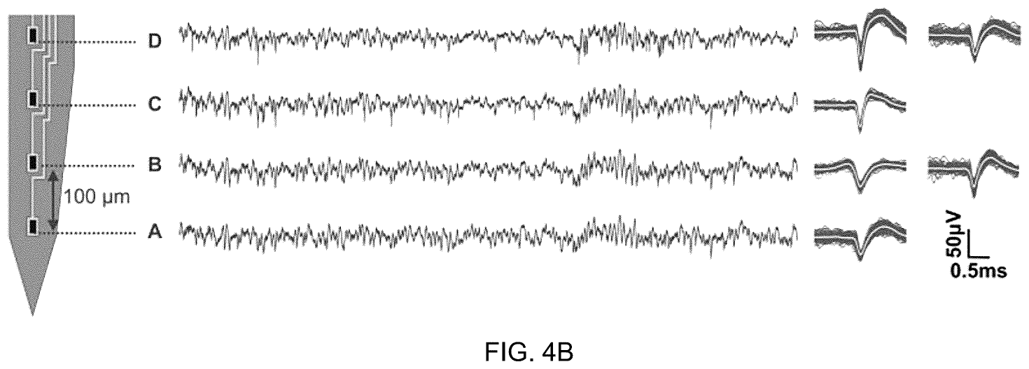

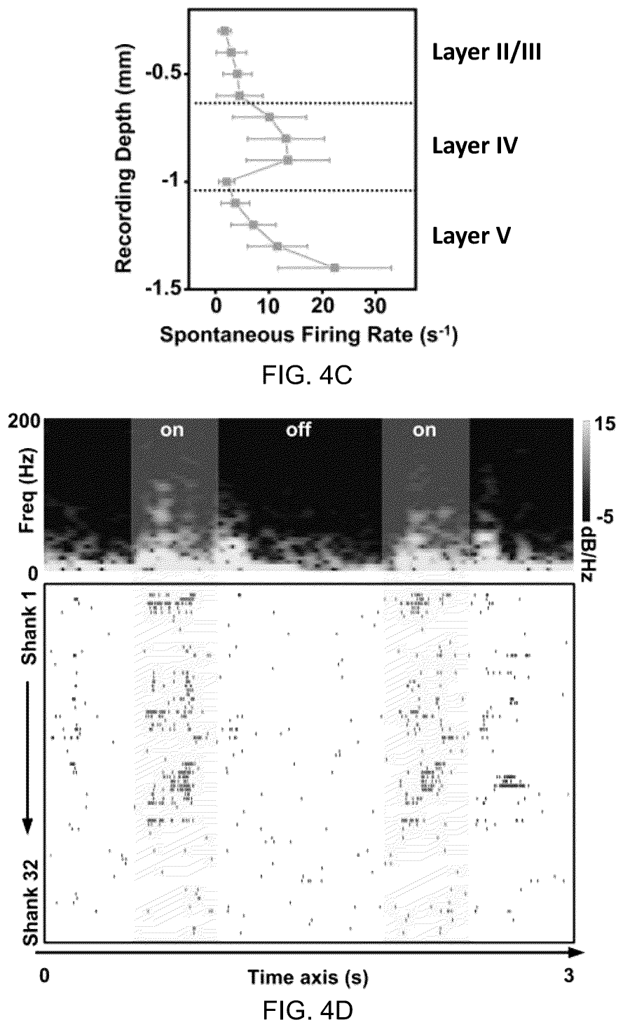

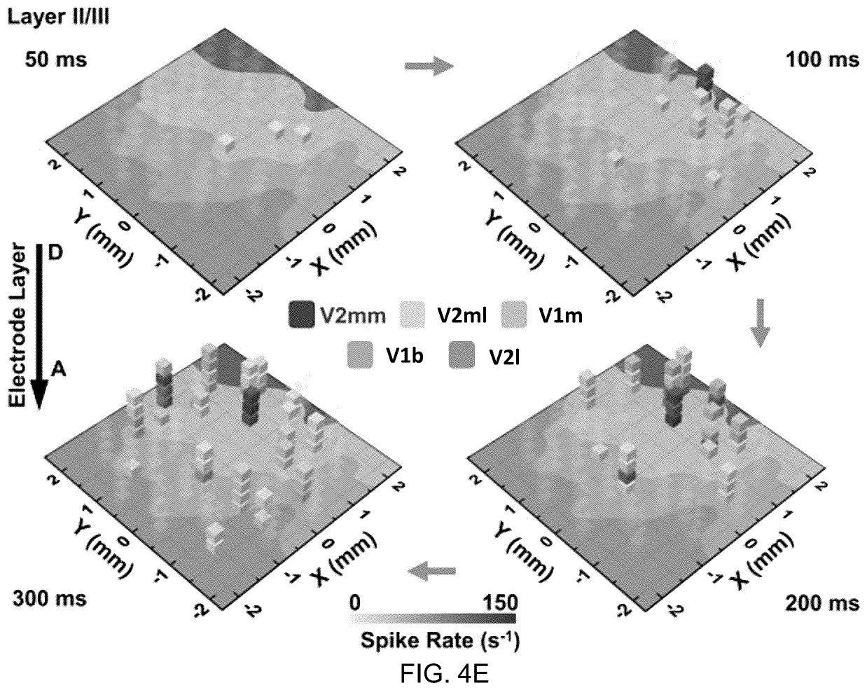

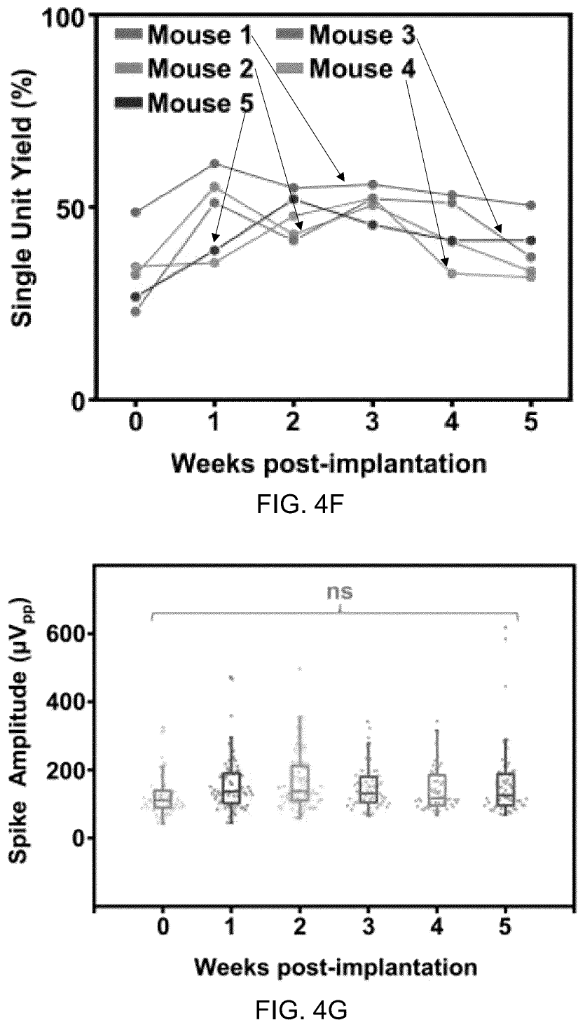

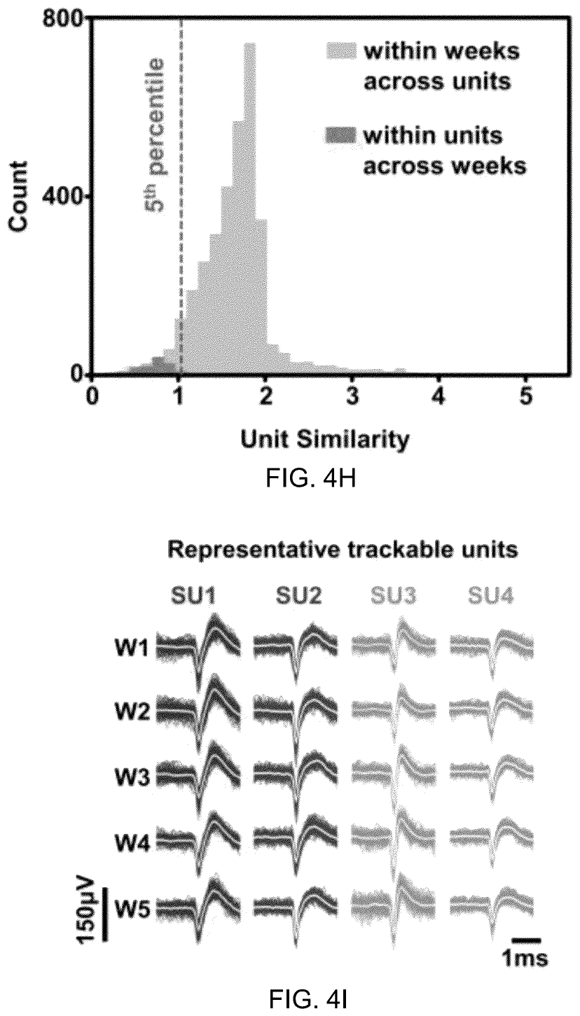

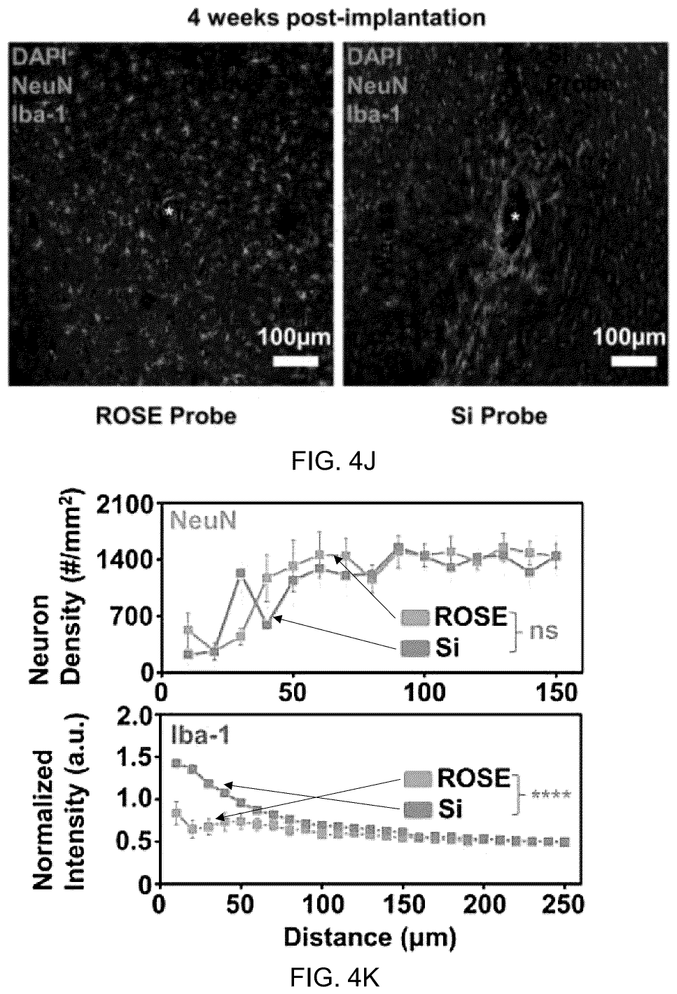

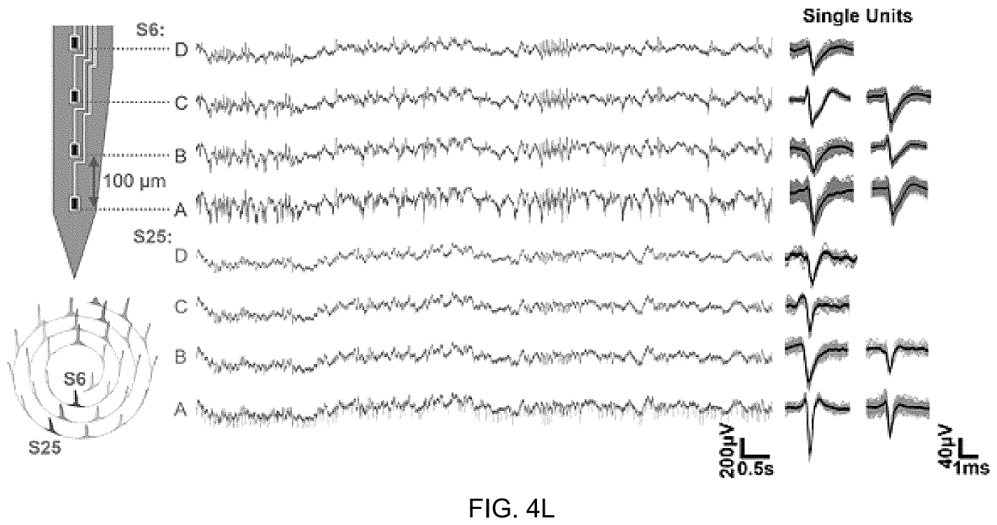

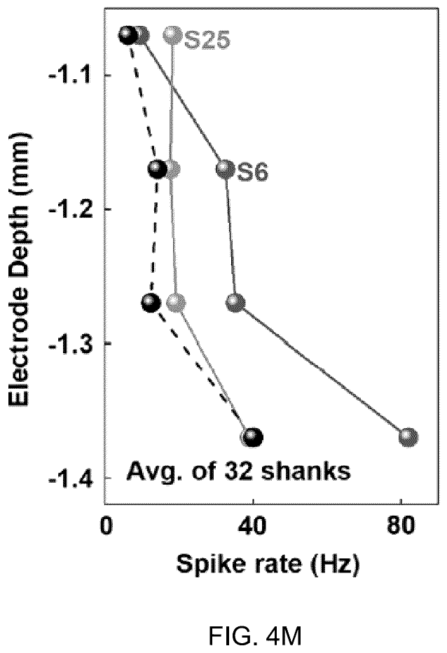

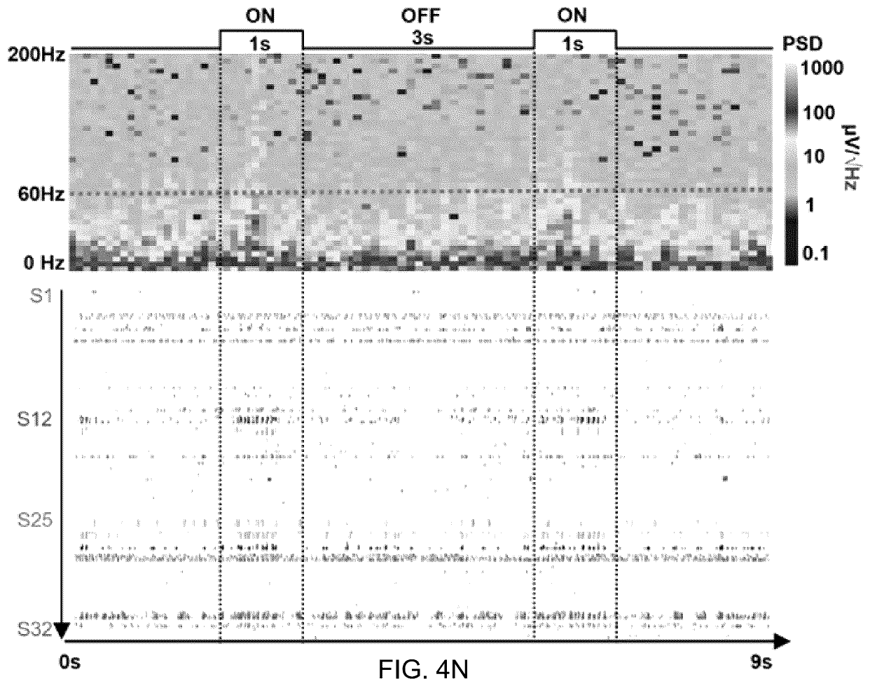

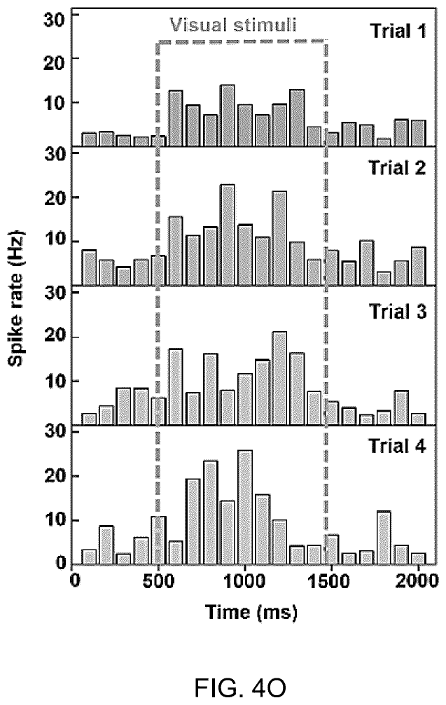

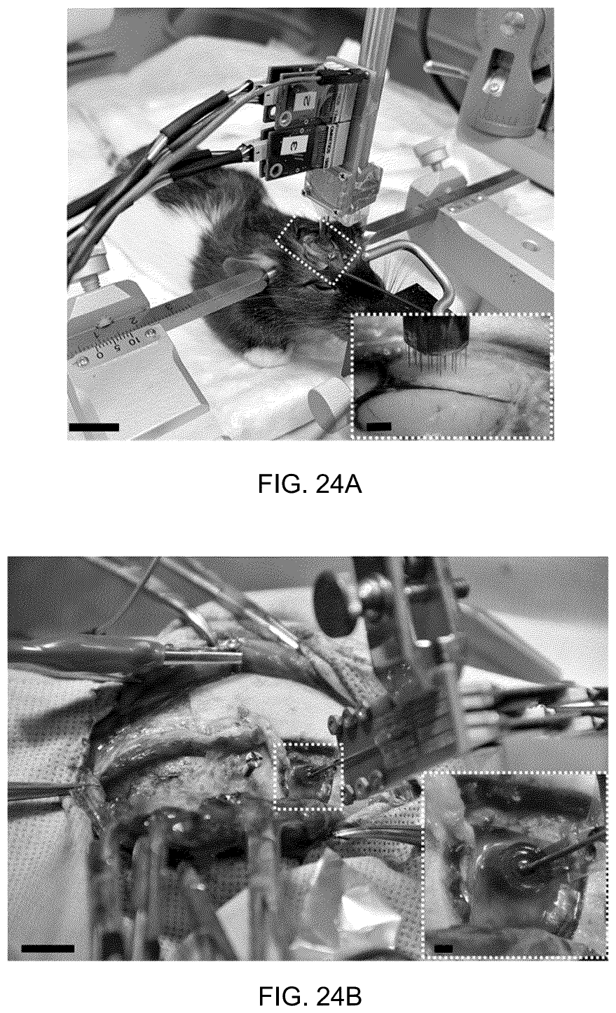

To validate the ROSE probe in vivo, an embodiment of a 128-ch ROSE probe (32 shanks with 4 electrodes per shank) was inserted into the rat visual cortex (FIGS. 4A and 24A). For this study, the shank thickness was 31 μm and the shank width was around 70 μm. This embodiment consistently penetrated rat brains with negligible bending or buckling, while the insertion tests indicated that with the same thickness, shanks as narrow as 30 μm can be inserted into rat brains without stiffening aids, showing the potential for further miniaturization. Meanwhile, ROSE probes can also potentially be made ultra-thin and coated with temporary stiffeners for the insertion.

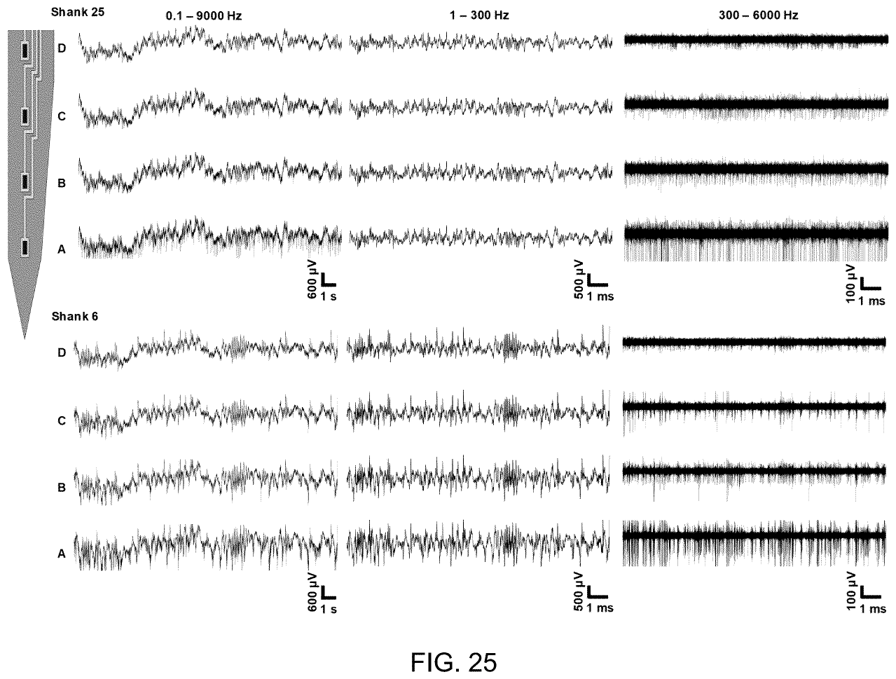

ROSE probes demonstrated high-fidelity electrophysiological recordings, capturing both local field potentials (LFP) and extracellular SU spikes (FIGS. 4B and 25). Generally, the recordings exhibited a noise floor of approximately 7 uVrms and captured approximately 1.2 well-isolated SUs per channel. When recording from different animals, the insertion depths were varied from 0.8 to 1.5 mm, which spanned a depth range from Layer II/III to Layer V. By averaging the spontaneous firing rate at various depths, a lower spontaneous firing rate was observed in Layer II/III, with relatively higher rates in Layer IV and V, except at the interface between these 2 layers (FIG. 4C). These observations are consistent with the previously reported laminar recordings in rat visual cortex, thereby verifying the precise placement of ROSE probes in the targeted locations.

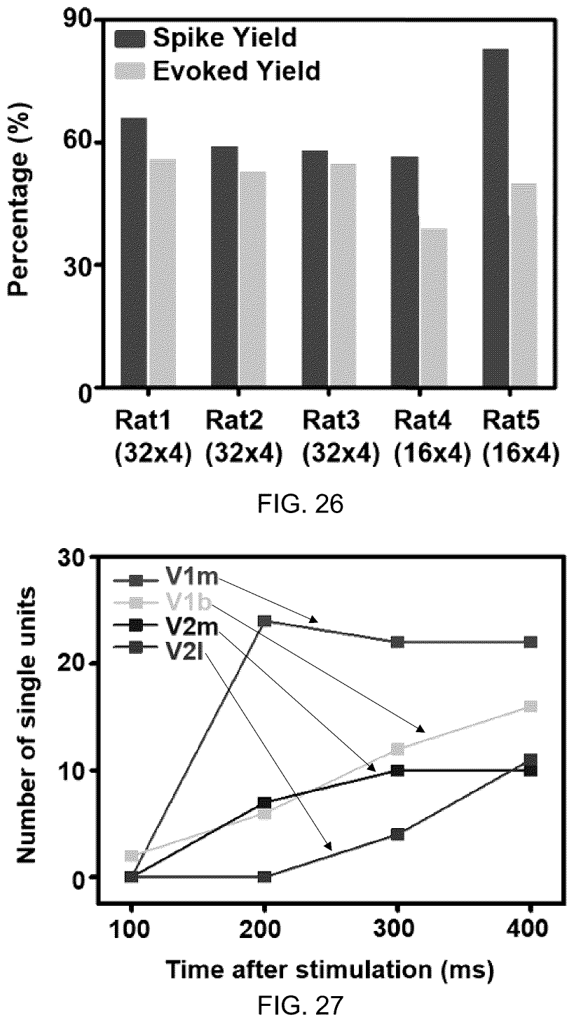

The ROSE probes also captured robust visually evoked responses. A typical response to visual stimuli includes increased gamma-band oscillations in LFP and a higher spike firing rate. From the visually evoked recordings, 79 SUs were isolated across 71 channels, while statistical analysis revealed that data in 64 channels exhibited significantly higher high-gamma power and increased spike firing rates following the onset of visual stimulation (FIG. 4D). Such a high-yield recording performance has been consistently achieved using ROSE probes, with a 64% SU yield on average (FIG. 26), a competitive performance with the leading results from state-of-the-art neural probes. The performance of ROSE probes in acute settings is particularly noteworthy given its large channel count and high shank number relative to the size of the rodent brain. For instance, when compared to recordings in rodents using 3D probes assembled from stacking, the ROSE probe demonstrated greater SU yield at a much higher shank density.

Embodiments of the ROSE probe may be monolithic and achieve simultaneous layer-specific and multi-regional SU recordings. In previous studies, shank-based neural probes are typically restricted to mapping spike activities within a linear or cross-sectional brain region. To capture higher dimensional information, multiple insertion sites with several independent probes are needed, but often with low probe density and requiring careful alignment and accurate electrode localization. Another way of realizing multi-regional recording is to assemble multiple planar probes together. However, it requires the stacking of multiple individual probes and their connecting PCBs. Meanwhile, monolithic devices such as the Utah array (UEA) have demonstrated compelling simplicity and have advanced in regulatory approval for human studies, but UEAs lack depth profiling and are limited by electrode density and throughput. For example, UEAs used for rodents are usually with only 4×4 channels. In contrast, embodiments of the ROSE probe are monolithic and have a flexible design that supports 128 high-density electrodes implanted in rodents simultaneously, covering a 3D volume of the brain while maintaining high-yield in vivo performance.

Based on the position of the implanted probe, the spike activities recorded in Layer II/III onto the visual cortex of a rat were mapped. Initially, a latency of about 100 ms was observed between the stimulation onset and the initiation of evoked responses in the monocular area of V1 (V1m). From 100 ms onward, a gradual increase in the number of spiking channels and spike firing rates was observed in the monocular area of the secondary visual cortex (V2m) and binocular area of V1 (V1b) (FIG. 4E). Eventually, at about 300 ms, the evoked spiking activity progressed to the lateral area of V2 (V2l). This phenomenon is consistent with the observation made previously by voltage-sensitive dye imaging that visually evoked events are usually initiated from V1m and then propagate in both directions to the V1 binocular area (V1b) and V2 (FIG. 27). To some extent, ROSE probe has demonstrated microscopy-like 3D spatiotemporal mapping of spike activities. The results indicate the potential use of the ROSE probe to enable high spatiotemporal resolution in three dimensions and multi-region hierarchical processing of neural activity.

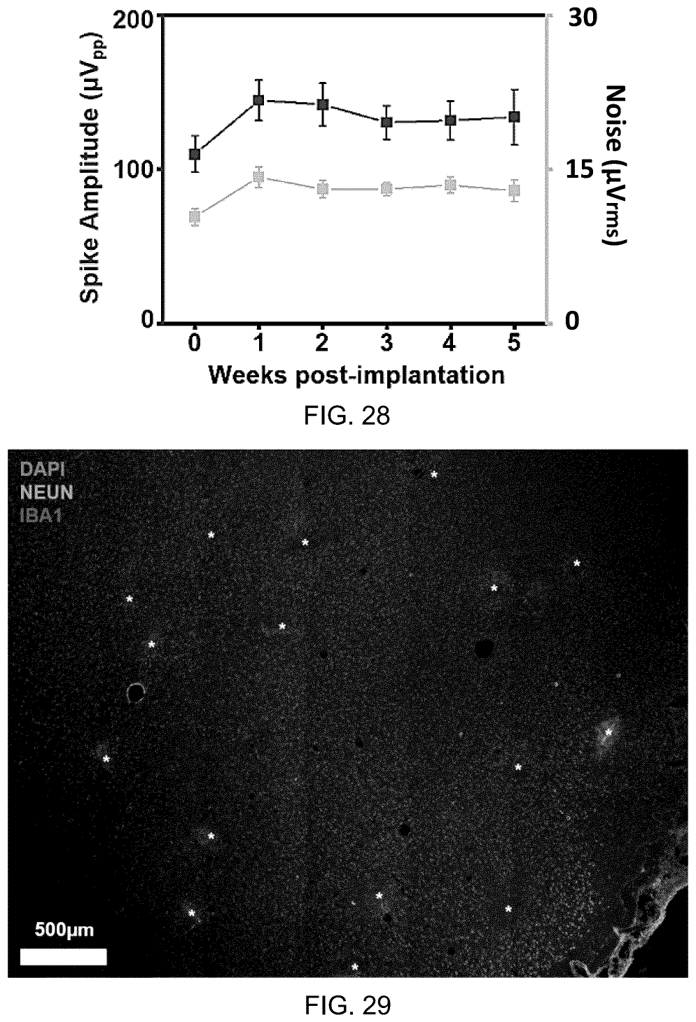

After achieving consistently high acute performance, the long-term recording capability of ROSE probes were investigated. Embodiments of a 16-shank, 128-channel ROSE probe were implanted into the visual cortex of mice for a semi-chronic recording study spanning 5 weeks (N=5). Similarly, visually evoked activities were collected over 5 consecutive weeks following the probe implantation. Overall, considering the initial number of functional electrodes in each probe, the SU yield across all 30 recording sessions was 43%±10% (mean±SD). The mean number of SUs identified per session was 45±13 (mean±SD). Upon analyzing the results, no decline in SU yield was observed between week 0 (33±10%, mean±SD) and week 5 (39±7%), with recording performance peaking at week 3 (51±4%) (FIG. 4F). Spike amplitude and the waveform shape are established metrics for assessing the stability of the electrode-tissue interface. Here, statistical analysis revealed no significant differences in spike amplitudes across 5 weeks from individual mice (FIG. 4G). The baseline noises also remained consistently low throughout all weeks and animals (FIG. 28). It is envisioned that a semi-chronic stability will enable numerous behavior studies in animals and even temporary use in humans. Previous studies indicate that chronic inflammation around microelectrodes persists, leading to neuronal cell death and process loss by 4 weeks post-implantation. Therefore, the stable SU yield, consistent spike amplitudes, and stably low noises observed over 5 weeks suggest significant promise for extended chronic recordings.

To further evaluate the validity and stability of these over-month-long recordings, a unit similarity analysis was conducted. Unit similarity was quantified using Mahalanobis distances (DM) between the centroids of SU clusters. The distribution of DM between all SU pairs represents the overall similarity of sorted units in each session (FIG. 4H). Across the 5 weeks of recordings from 5 animals, the 5th percentile of these distributions consistently exceeded 1, suggesting a good separation of the sorted and curated SUs. Such a similarity measure is critical, especially in chronic recordings, to ensure the reliability of recorded SUs, as probe shunting in the brain can lead to overcounting of units. Using the 5th percentile of the resulted distribution as a threshold, trackable SUs from week 1 to week 5 with consistent spike amplitudes and waveforms (FIG. 4I) were identified. The ability to maintain stable, month-long SU recordings facilitates many long-term neuroscience studies, as experiments investigating sensory responses or behavioral tasks often rely on stable SU activities over weeks to uncover novel insights. These stable SU recordings were made possible through numerous iterations of ROSE probes and in vivo neural engineering efforts.

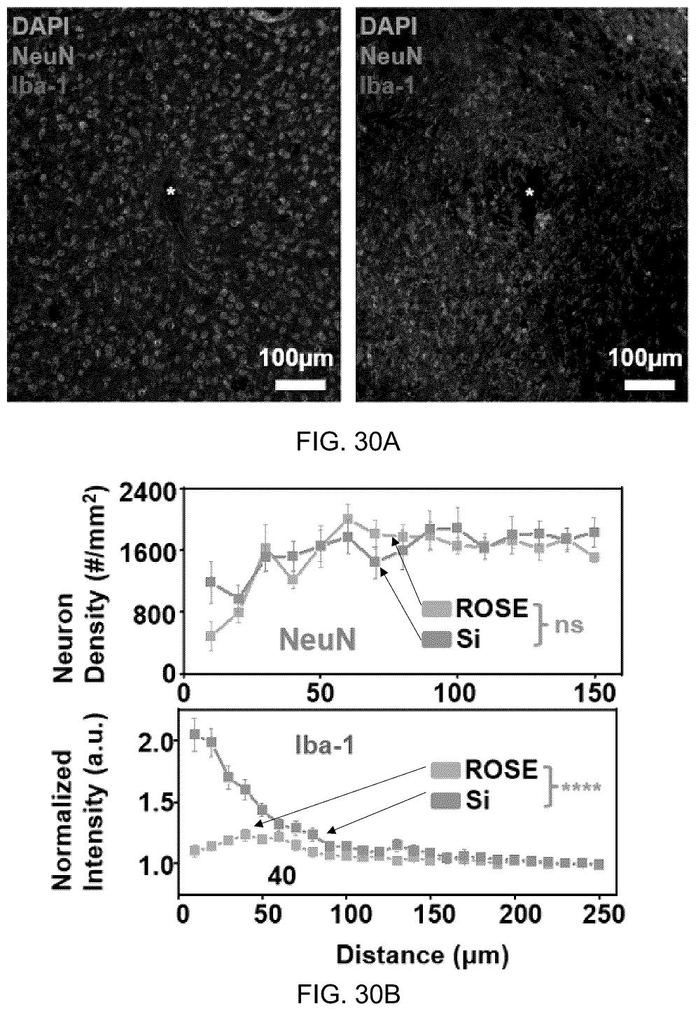

It is hypothesized that the over-month-long recording stability is attributed to the flexibility of the ROSE shanks. To investigate it quantitatively, a histological study was conducted for up to 4 weeks comparing the cortical tissue reactions to both ROSE probes and a traditional Michigan-style Si probe (NeuroNexus) (FIG. 29). Representative histology images of the tissue around the probe show that embodiments of the ROSE probe resulted in similar neuron density in both the 1-week and 4-week brain samples compared to the traditional Si probes, with no statistical significance found when comparing individual bins between two groups (FIGS. 4J and 30A). For Iba-1 marked microglia intensity, although both groups have elevated microglia presence within tissue approximately 100 μm from the probe hole, ROSE group exhibits overall lower microglia intensity than the Si group (FIGS. 4K and 30B). As first responders in the brain, microglia are recruited to the implant site as a response to the implant injury and the presence of the foreign body. Higher Iba-1 expression usually indicates more microglia accumulation and activation and is commonly associated with more tissue damage and inflammation. Based on this comprehensive study, it was concluded that the flexibility of the planar component of the ROSE probe allows for less tissue stress, thereby reducing inflammatory reaction compared to traditional stiff Si probes.

While 16 shanks appear to be the current maximum for rodent arrays, higher shank numbers may used and may be required for NHP and human applications. To shed light on the potential brain damage caused by a scaled-up version of the ROSE probe, tissue volumetric displacement analysis was conducted in an ex vivo pig brain (Example 10). Overall, comparable insertion footprints were observed to the probe design, suggesting a minimal tissue disruption caused by the implantation of a 256-shank ROSE probe.

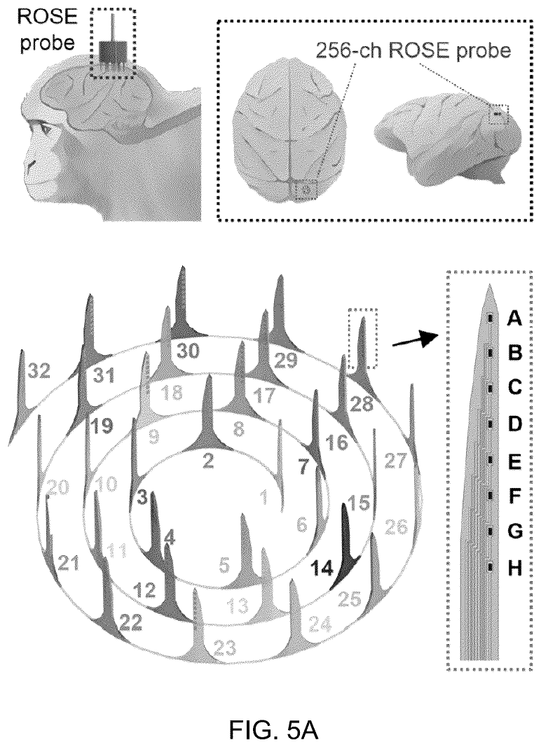

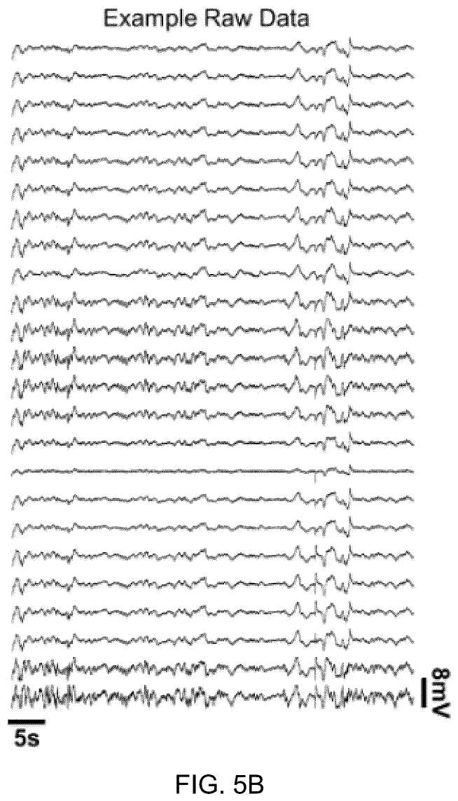

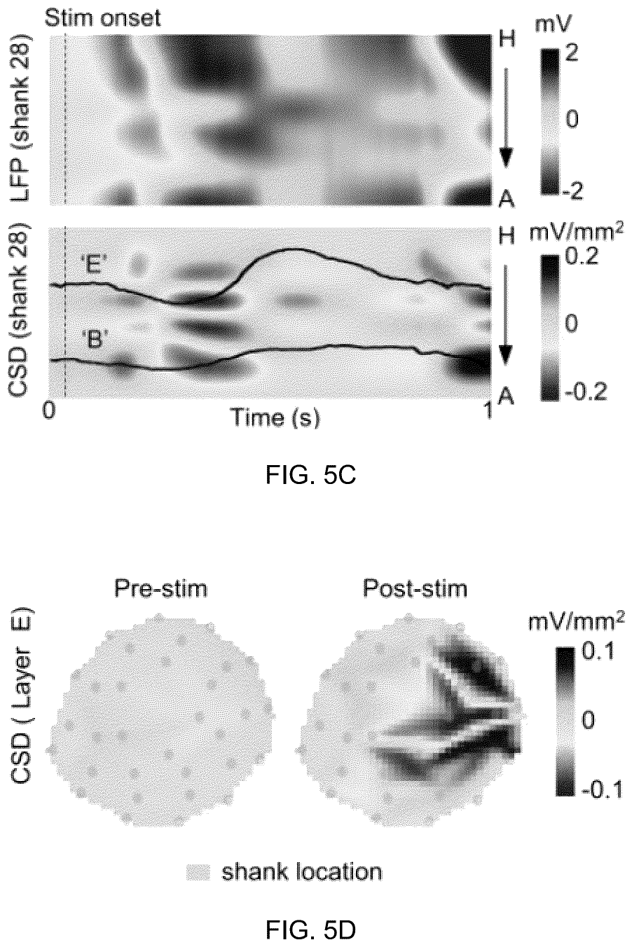

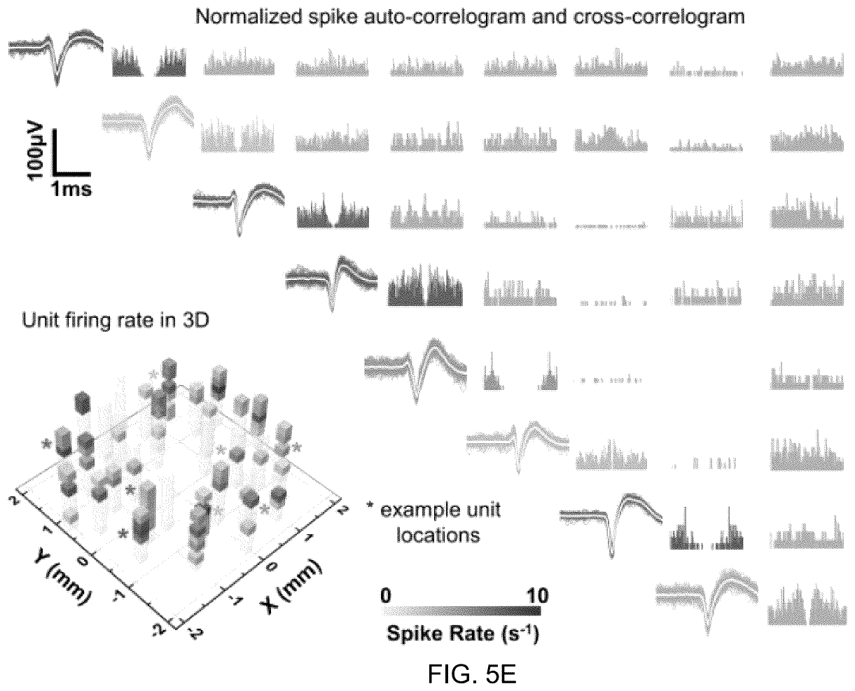

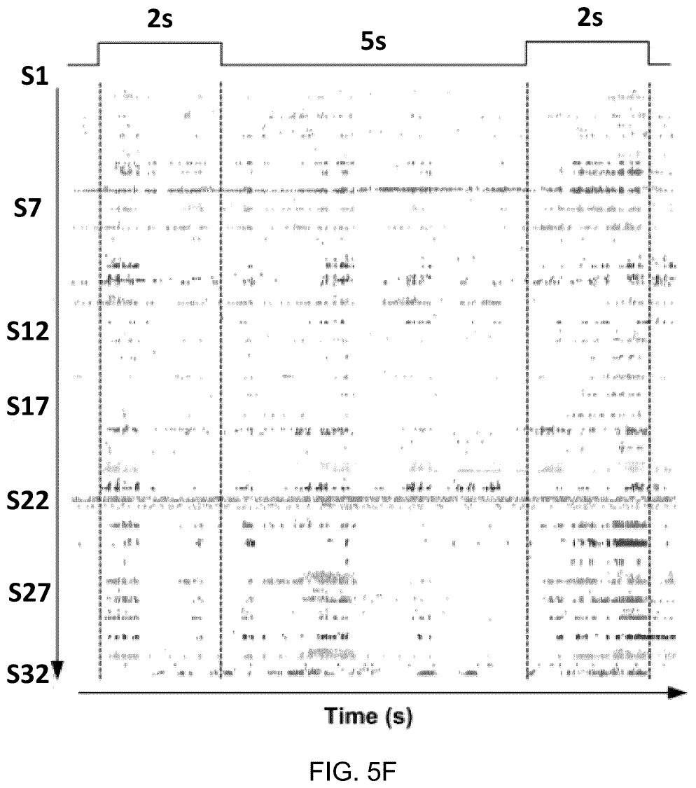

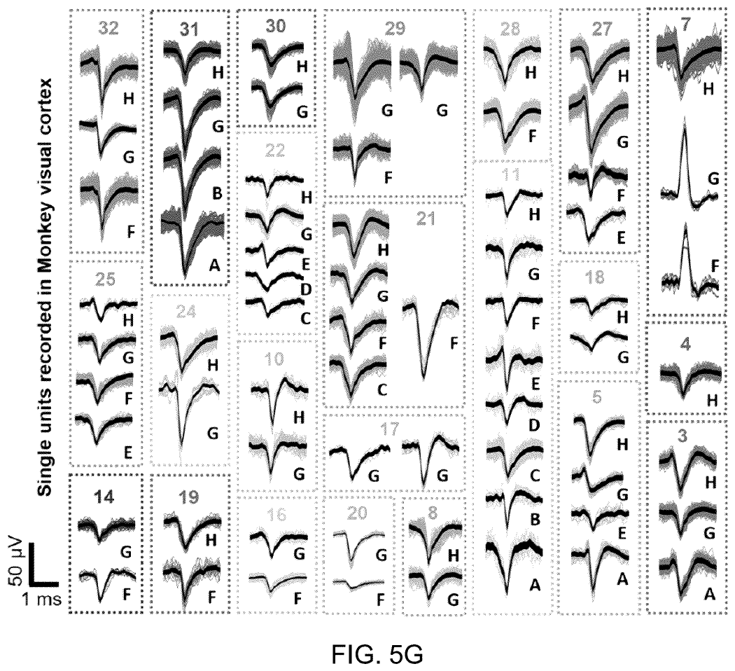

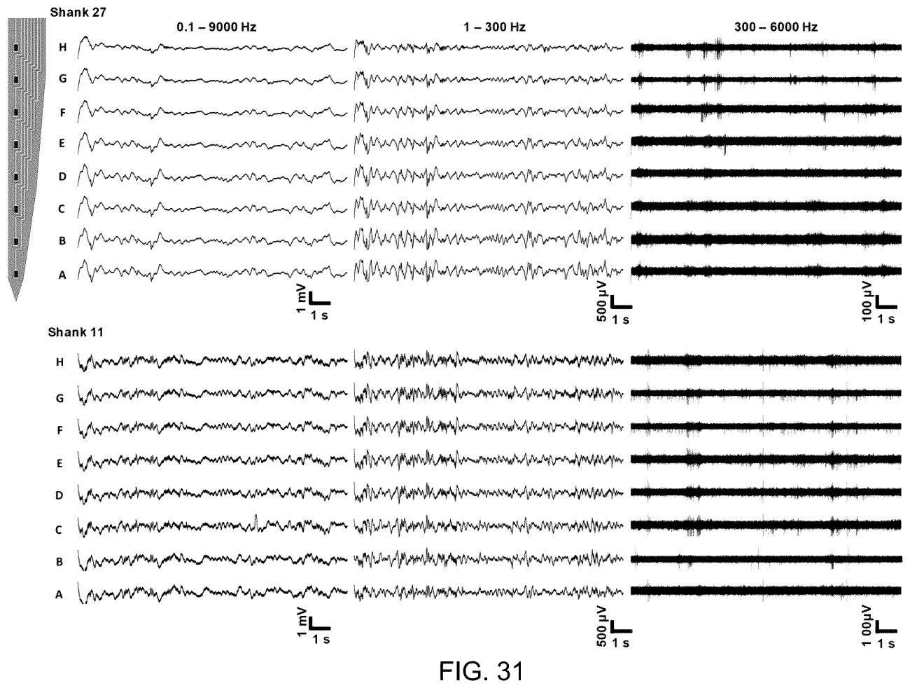

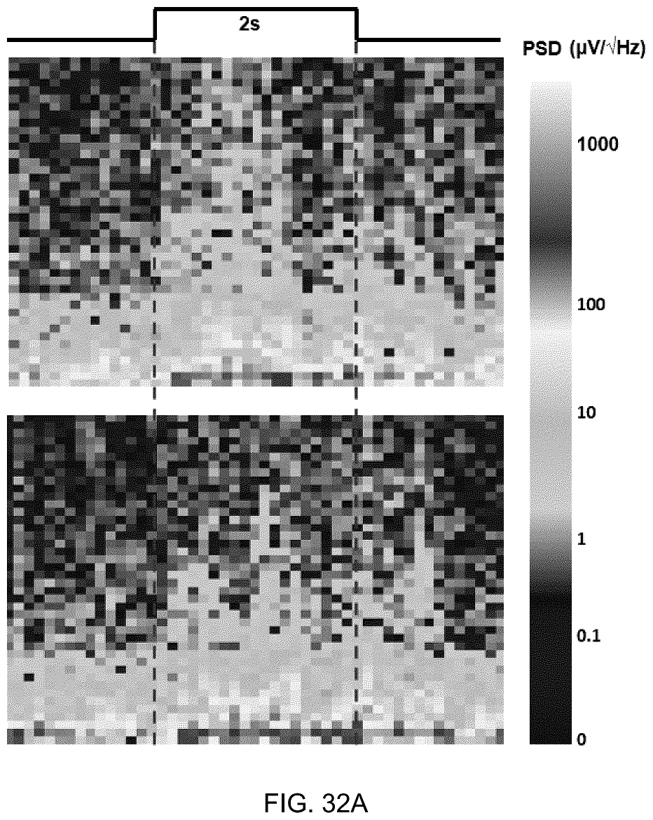

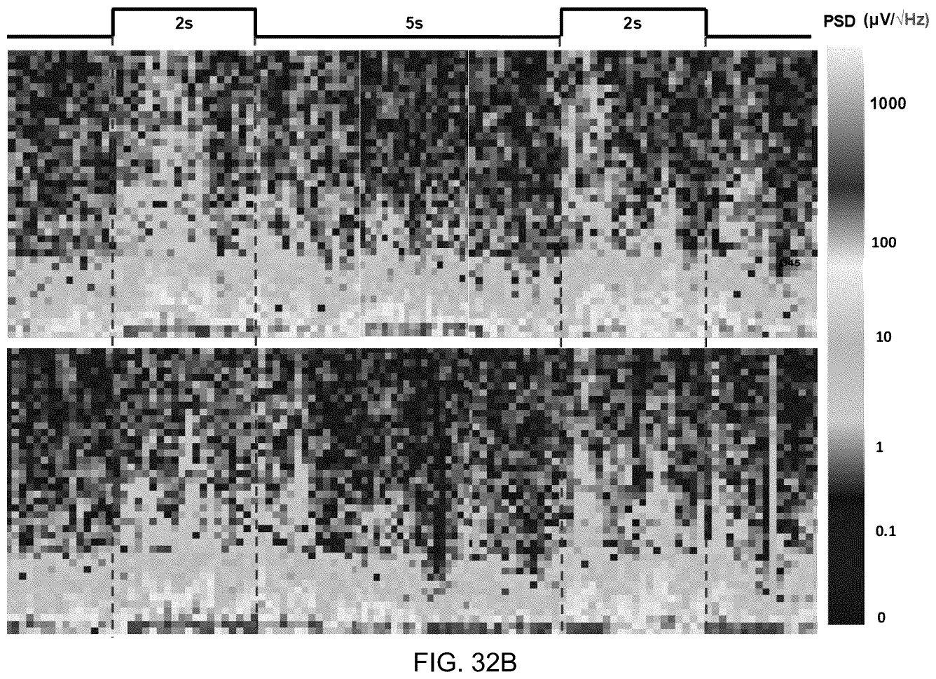

In addition to rodents, a large-scale ROSE probe embodiment (256-channel with 32 shanks and eight electrodes per shank) was tested in the visually responsive cortex (V4/Inferior Parietal Lobule) of a rhesus macaque monkey (FIGS. 5A and 24B). Similarly, the recorded signals were analyzed across multiple frequency bands (FIGS. 5B and 31). The 256-channel ROSE probe provided a high-resolution 3D view of visually evoked LFP responses (FIGS. 5C and 32A). Each shank's 8 electrodes captured laminar profile of the evoked LFPs, revealing a detailed picture of current flow through multiple cortical depths (FIG. 5C). By examining these laminar profiles, the peak evoked response was localized and identified as a region of strong current sinks at electrode layer E. Thus, 2D current source density (CSD) plots focused on the specific electrode layer (FIG. 5D) were constructed, which highlights how stimulus-driven currents propagate across the cortical surface. This spatial visualization underscores the complexity of cortical circuitry and the advantage of the ROSE probe's dense 3D sampling capabilities.

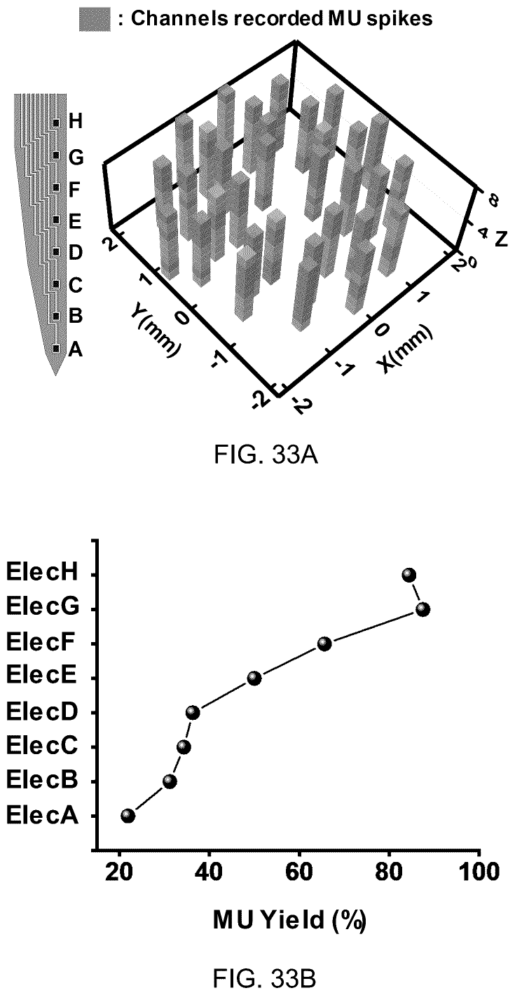

Beyond LFP, the ROSE probe also demonstrates high-yield SU and MU detections. The large-scale probe captured SUs across a 3D configuration with distinct spiking patterns (FIG. 5E). To summarize, the ROSE probe accomplished a 69% multi-unit (MU) yield and 44% SU yield out of the 223 functional channels in a total 20-minute recording (FIGS. 33A-33B). The MU band signals also confirmed the robust visually-evoked responses with a 61% evoked MU yield (FIG. 5F), consistent with the known visual responsiveness of V4/IPL. Together, these results, along with the rodent experiments, highlight the ROSE probe's ability to reliably capture high-quality, 3D electrophysiological signals across multiple frequency bands in both small and large animal models.

In summary, this example demonstrated an embodiment of the ROSE method that directly transforms conventional, planar soft electronics into the first monolithic 3D neural ROSE probes by deterministic rolling. Insertion studies confirmed that mm-length-scale shanks can penetrate the brain without any temporary stiffening, and revealed the slipping-induced insertion failure mechanism, providing guidelines for ROSE methods and ROSE probes. Layer-specific, multi-regional recordings in rodents and NHP models further validated ROSE probes with high in vivo performance and stability.

Embodiments of the monolithic 3D soft ROSE probes may enable several important neuroscientific advances through microscopy-like spatiotemporal spike mappings. For instance, in brain regions such as the prefrontal cortex, neurons extend over many mm, both parallel and perpendicular to the cortical surface, making the 3D recording approach crucial for studying interlayer and interpatch relationships. Moreover, the 3D nature of recordings will allow more precise BCIs by sampling neural activity from both superficial and deep layers, as well as sulcal regions. Due to its design flexibility, the ROSE method allows for customizable 3D geometries to serve specific recording needs, in a way similar to 3D printing. It also provides a unique and highly deployable pathway towards MEA scaling. For example, in an embodiment the ROSE method may be applicable to advanced flexible electronics integrated with sophisticated complementary metal-oxide-semiconductor (CMOS) technologies to achieve very large-scale integrated 3D ROSE probes, with thousands and tens of thousands of electrodes in miniaturized footprints. Further, it may be possible to achieve optoelectronic-shank-based 3D ROSE probes, enabling multimodal, closed-loop brain interrogation from combining electrophysiology and optogenetics.

Another inherent nature of the ROSE probe is the softness of its electrode shanks. This property can be further exploited to enable clinical applications such as neuroprosthetics. Flexible probes fabricated from polymer substrates, such as polyimide, Parylene C, and PDMS, have provided better mechanical compliance to achieve more reliable neural interfaces. Noteworthy, even though the ROSE probe is flexible, several experiments with low-yield brain recordings were observed, especially when the shank width is wider than 130 μm, presumably due to the large volumetric displacement during the probe implantation.

The overall design of planar component included 5 layers with Kapton/polyimide (PI) bilayer as a substrate, Au/PEDOT: PSS bilayer as electrodes and interconnects, and SU-8 as an encapsulation layer. All shanks are designed to have a tapered profile with a tip angle of 20° to facilitate insertion and minimize tissue damage. There are four 10× 20 μm² microelectrode sites in each shank, with 50 or 100 μm electrode pitch and 90 μm distance from the shank tip. The shank width is 65-70 μm at the widest point, and the entire length of the shank is 1.6 mm. The overall thickness of each shank is 31 μm. Fabrication steps of the planar component include the following. (i) A Kapton film (25 μm) was laminated on a glass slide that was pre-spin-coated with 10:1 PDMS (30 μm). (ii) A PI layer (5 μm; PI-2545) was spin-coated on the Kapton surface to improve the smoothness, and the Kapton/PI substrate was then cured in N₂ atmosphere at 250° C. for 70 minutes. (iii) Cr (5 nm)/Au (100 nm) was deposited on the substrate by electron-beam evaporation, where Cr acted as an adhesion layer between the PI and Au layers. (iv) Photoresist (PR, S1805 G2) were used to pattern and define 3.5 μm resolution interconnection. For 2.5 μm feature size, lift-off resist (LOR 1A, 150 nm thick) and positive photo resist (PR) (S1813, 1.3 μm thick) were used. (v) Negative photoresist (SU-8 2005, 4.5 μm thick) was spin-coated and patterned on top of the substrate for encapsulation and hard baked at 180° C. for 30 minutes. (vi) Poly(3,4-ethylenedioxythiophene) polystyrene sulfonate (PEDOT: PSS) was electrochemically deposited using potentiostat. (vii) After PEDOT: PSS deposition, samples were rinsed with DI water and coated with Dextran (4:1 ratio with DI water), serving as a protective layer for the laser beam. A laser cutter (U4, LPKF) was used for defining the outline of the shank. After laser cutting, the dextran was rinsed with DI water. Laser-cut samples were soaked in acetone for easy peel-off from the PDMS spin-coated substrate and lifted off with a tweezer. Samples were rinsed with IPA and DI water and dried at ambient temperature for 30 minutes.

Prior to the rolling, a supporting micro-mandrel was glued on the side further away from the interconnect end of the planar component by medical adhesive (3M Vetbond). A 400-μm-thick, 2-mm-width PDMS slab with a 10:1 curing agent ratio was bonded to the base region of the planar component to serve as the spacer among the rolled loops (the right edge of the slab was usually placed under the position of the first shank). The rolling started by slowly rotating the micro-mandrel counterclockwise toward the interconnect end of the planar component. The rolling machine was customized to aid the rolling process. A complete ROSE process typically takes about 5 minutes. A Kapton tape attached to the backside of the planar component (FIG. 6A) was used to secure the final ROSE structure. In other embodiments, any medical tape, adhesive, or epoxy may be used instead of Kapton tape. The supporting micro-mandrel in the center of the ROSE probe can be removed by sonication for 20 seconds in acetone if needed (FIG. 6B).

A 0.6% agarose gel (Agarose BP160-100) was prepared for a brain phantom. The gel powder and deionized water were mixed and stirred for a few hours at 140° C. until the solution became transparent.

The as-prepared solution was then poured into a cylinder container with 5.5 cm in diameter and 1 cm in height. The solution was cooled down at room temperature overnight and formed the gel. No coatings were used to stiffen the shanks and the interconnect part. The shanks were slowly inserted with a speed of 0.5 mm/min into the agarose gel to study the insertion mechanism by a test stand (Mark-10). The force was measured by a force gauge (model M5-012), which had a data resolution of 0.1 mN. The cyclic insertion test was performed using LTS150/M Long Travel Stage (Thor Labs), where the gel was fixed on the stage and the probe on the moving platform. The displacement from the gel contact to insertion was about 1 mm. The moving speed of the platform was 0.5 mm/s, and each cycle took 8 seconds. The probe was rinsed with DI water and the impedance was measured by every 100 insertion cycles. For the planar component, the probe base region was attached to a glass slide to prevent the probe from bending.

The procedures with rats were approved by the Animal Care and Use Committee of Dartmouth College. Male Long Evans rats with an age of 10-12 weeks (300-350 g, Charles River) were used for the craniotomy. All rats were kept in 12 hours of the night-day cycle. No other experiments were done on the animals, and all of them were kept under the same condition. All surgical tools and environments were sterilized before the surgery. Before the craniotomy, the rat was anesthetized with 2˜3% inhalant isoflurane (ISO) and 1.5% Oxygen gas in the induction chamber. Once fully anesthetized, the rat was transferred to a stereotaxic frame, and its head was fixed with non-traumatizing ear bars. The rat was placed on a heating pad at 37° C. for entire surgery and continuously received 1˜2% of ISO and 2% Oxygen gas through a nose cone connected with a charcoal canister for purification purposes. Eye lubricant was applied to prevent the retinas from drying. Hair between the eyes to ears was shaved and the exposed scalp was cleaned with three times of betadine and 70% IPA.

A clean straight incision was created using an 11-blade scalpel, and the scalp was held apart with hemostats. Fascia and blood were removed using sterile saline and sterile cotton swabs until a clear skull with the bregma and the lambda were visible. To create a cranial window on a visual cortex, a 5.0-mm rectangular craniotomy was drilled centered at 7.0-mm posterior from bregma and 3.5-mm lateral from the midsagittal line. A drill bit did not stay at a specific location for more than 2 seconds to minimize/avoid heat damage on the vessels. The entire skull was kept moist and cooled with frequent saline wash, and the bone debris was wiped with sterile cotton swabs and Kim wipes. With gradual drilling, the bone flap was removed from the skull, avoiding any damage to the cortex. Any bone residues and debris were cleaned with multiple cleaning with sterile saline with sterile cotton swab and forceps. For durotomy, a 30-gauge syringe needle was used to make incision at an edge of the dura which was then carefully removed by forceps without damaging cortex or intact blood vessels nearby. Dura was peeled off to minimize the dimpling effect when inserting a probe. No major bleeding was observed even after the dura was peeled off, and any bleeding was fully controlled before the insertion.

For testing ROSE probe functionality in anesthetized rhesus monkeys, aseptic surgical procedures were performed for probe insertion and recording of spontaneous and evoked activity in the visual cortex in a female monkey (20-22 years). These monkeys were acquired as part of a larger study in aging at Boston University from a private vendor with complete health records. Monkeys were housed individually in the Laboratory Animal Science Center (LASC) at Boston University School of Medicine (BUSM), which is fully accredited by the Association for Assessment and Accreditation of Laboratory Animal Care (AAALAC). All procedures were approved by the Boston University Institutional Animal Care and Use Committee (IACUC) and were conducted in accordance with the guidelines established by the NIH Guide for the Care and Use of Laboratory Animals. Each monkey was sedated with ketamine hydrochloride (IM, 10 mg/kg) and anesthetized with propofol (IV, 0.3-0.4 mg/kg/min) to effect. The head was stabilized in a stereotactic apparatus, and, after skin incision and reflection of the temporalis muscle, a small burr hole (approximately 5 mm×5 mm) was drilled over the right occipital lobe. The dura was incised to expose the dorsal surface of V4/parietal cortex, and the ROSE probe was inserted using a micromanipulator mounted on a stereotaxic machine as described below. At the completion of recording, the monkeys were euthanized by exsanguination while deeply anesthetized and the brain transcranial perfused and harvested.

The procedures involving mice were approved by the Institutional Animal Care and Use Committee of Weill Cornell Medical College. Female C57BL/6 mice aged 9-12 months (25-35 g, Jackson Laboratory) were used for craniotomy, probe implantation, and subsequent recordings. All mice were maintained on a 12-hour light/dark cycle. No other experiments were conducted on these animals. Each mouse was anesthetized with 5% isoflurane for induction and maintained with 1%-2% isoflurane. Intraoperative vital signs were monitored using a mouse oximeter (Starr Life Sciences, PA). Body temperature was maintained at 37° C. using a regulated heating blanket (RWD Life Science, TX, USA). A 4-mm cranial window was opened over the visual cortex, centered 2.5 mm lateral to the midline and 3 mm posterior to the bregma. The dura was left intact. The cranial window was subsequently covered with sterilized polydimethylsiloxane (PDMS) film and sealed with dental cement. A head plate was affixed over the cranium. Two weeks after surgery, the mice received a 3-day long habituation training session for head fixation.

The procedures used for semi-chronic mice surgeries were approved by the Animal Care and Use Committee of Dartmouth College. Both male and female C57BL/6 (10˜13 weeks old, Charles River) were used for the study. All animals were housed in 12-hour light/dark cycle with ad libitum access to the food and water. 4 hours prior to surgery, dexamethasone (2 mg/kg; i.p.) was injected to animals to prevent edema and reduce inflammatory response. All tools for surgery were sterilized and surgery area was cleaned with 70% Ethanol. Mice were placed in an induction chamber with 2% oxygen with 2% isoflurane. Hair was shaved from between the eyes to ears and eye ointment was applied to prevent eyes from drying. Mice were positioned on a stereotaxic frame with continuous flow of 1-1.5% of isoflurane through the nose cone. Once mice are under stable surgical plane, clean midline cut was made following the mid sagittal line. Exposed skull was cleaned with sterile cotton swab and/or back side of scalpel blade. About 4 mm-by-4 mm cranial window (center at AP: −3.5 mm, ML: 2.5 mm) was opened. Continuous irrigation with chilled saline was applied to reduce accumulated heat from drilling and drill bit was not stayed in one position more than 2 seconds. Cranial window was covered with soft silicone gel (dura-gel) followed by Kwik-sil (WPI). Once the skull was completely dried, chamber base was anchored on the skull with dental cement (3M). After full chamber assembly, at least one week of recovery is waited prior to probe implantation.

For anesthetized rodent models, the ROSE probe was first connected to the MagMatrix connector which was then connected with all necessary cables. The ROSE probe was positioned to be perpendicular to the visual cortex. The probe was inserted into target depth of 700 μm˜1500 μm automatically with speed of 0.05 mm/s (NeuralGlider). No bleeding was observed during and after the insertion. In acute settings, the inserted cortical region with shanks were covered with Kwik-Sil or Kwik-Cast, while in semi-chronic settings, a thin-layer of silicone gel (Dow Corning) was first applied between shanks to fix the positions, then kwik-sil was used to seal the cranial window, followed by dental cement to secure the entire probe with brain. For the macaque monkey models, the ROSE probe was tightly connected to an arm of the monkey stereotaxic frame which was rotated to have perpendicular angle to the visual cortex via the burr hole. The probe was inserted into the cortex with a speed of 0.5 mm/min for 500 the first 50 μm, and then stopped for 1˜2 minutes. The same insertion method was continued, and the final depth of the probe insertion was 1.5 mm. For the awake, head-fixed mouse model, on the day of recording, the mouse was briefly anesthetized with isoflurane for PDMS film removal. A NeuralGlider inserter was used to position the probe perpendicular to the cortex and insert the 16-shank ROSE probe with 0.5 mm/min. The insertion was performed carefully to avoid large blood vessels. Once insertion finished, 5-10 minutes were waited before sealing the craniotomy with Kwik-Cast and dental cement. The insertion depth was calibrated to 1.3 mm to ensure coverage of cortical layers II-VI.

For the one-week and four-week histology study, single-shank non-functional A-style Si probes (3 mm length, NeuroNexus, Ann Arbor, MI) and non-functional ROSE probes were used. The footprint of a single-shank Si probe (thickness 10 μm, width 150 μm) is comparable to a single shank in the ROSE probe (thickness 31 μm, width 70 μm). One or four weeks after the dummy probes were implanted, animals were deeply anesthetized with an overdose cocktail of Ketamine (90 mg/kg) and Xylazine (9 mg/kg) and perfused with 300 ml of 1×PBS followed by 300 ml 4% paraformaldehyde (PFA) in 1×PBS. The bottom of the animal skull was removed and post-fixed in 4% PFA overnight. The brain was then removed from the skull, dehydrated in 15% and 30% sucrose sequentially, and frozen in optical cutting temperature compound (OCT). Then the frozen tissue was cut into 25 μm sections on a cryostat. The slices were rehydrated in citrate buffer and then blocked with 10% goat serum, followed by permeation treatment with 0.1% Triton-x for 45 minutes. Then the slices were stained with antibodies including NeuN (Millipore mouse 1:250), Iba-1 (Millipore rabbit 1:500), GFAP (DAKO chicken 1:500) and Hoechst counter stain. Slices were imaged on a confocal microscope (Olympus Fluoview 3000) at 20× magnification and the images were analyzed with a custom MATLAB script. 25 concentric bins (each 10 μm wide) were created around the probe implant. To quantify the tissue response, the grayscale fluorescent intensity of the cellular markers were calculated within each 10 μm wide concentric bin that centered on the probe hole. In both conditions, there is decreased neuron density in bins adjacent to the probe, and at approximately 50 μm from the probe hole, the density returns to the baseline level similar to distal regions. The fluorescence intensity of the stain or cell counts within each bin was quantified. To control for the variability of gray scale intensity, 5% of the area at each corner of the images (total 20%) were chosen as control region and the quantified intensities or cell counts were normalized to the control region. To calculate the background noise intensity within the control images, pixels greater than 1 SD above the mean are considered as meaningful fluorescence and were excluded from calculation. Then the background noise intensity threshold was calculated as one SD below the mean of remaining pixels. Once the background intensity threshold was determined, all pixels with intensity above the background were used for analysis and then plotted as a function of distance. For NeuN, the neurons were manually counted within each bin using the ImageJ multi-point tool.

For acute rat recordings, the animal was placed on a water-based heating pad during the entire recording to avoid extra electromagnetic noise. Electrophysiology data were first recorded during spontaneous conditions in a dark room for 10 minutes and then during visually evoked sessions for another 10 minutes. The visual stimuli were generated using the Psychtoolbox in MATLAB and presented with a 24″ LCD monitor to the eye contralateral to the implant (30 cm from the eye). The stimuli (‘ON’ state) are a randomly ordered sequence of moving sinewave, black-and-white gratings (2 cycles per second, 3 cm wide bars at 100% contrast, 120 Hz) with eight angles (0°, 45°, 90°, 135°, 180°, 225°, 270°, 315°). Each stimulus lasts 0.5 seconds followed by another short period (0.5-1.5 seconds, randomly) of a blank gray screen (‘OFF’ state). An Arduino Mega 2560 was used with the MATLAB-based Psychtoolbox to synchronize visual stimuli and electrophysiology recording. For awake mouse recordings, the animals were head-fixed and placed on a treadmill. The visual stimulation setup is the same as described above, with 1-second stimulation time. For the decoding purpose, 24 angles (15° step) of gratings were presented in a random sequence as one session, and recorded brain activities under 60 sessions with 30 seconds resting time between each session, forming a dataset of 1440 simulation trials. For acute monkey recordings, the heating pad underneath the monkey was disconnected to minimize the environmental noise after the 15-minute waiting period. 10-minute spontaneous recording and about 10-minute of recordings under the same visual stimulation described for rats were conducted, but with a longer stimulation (2 seconds) and resting period (5 seconds). While the eyes of rats are naturally open during anesthesia, the monkey eyelids were manually opened and closed on and off for each 30s. A substantial difference was found between MU and SU in the monkey brain recording. It is hypothesized that this discrepancy came from electrodes at shank tips entering the bottom of visual cortex, as spike sorting revealed that the tip electrodes (A-C) had a low percentage of well-isolated SUs, suggesting that those microelectrodes are not positioned near neuronal somas. When excluding the electrodes A-C, the SU yield increases to 58%, while the MU yield remains similar at 70%.

The implanted probes measure 71 μm in shank width and 31 μm in shank thickness, with 16 shanks distributed within a 2.2 mm diameter. Before implantation, the probes were sanitized by immersing in 70% Ethanol for over 15 minutes. A custom 3D-printed chamber was installed on the animal head and secured by dental cement to protect the implanted probes. By leveraging the reusable MagMatrix PCB connector, only the probe and a lightweight acrylic top board (<2 g total) remain on the animal head when recordings are not in progress. For the weekly recording session, animals were lightly anesthetized to perform visually evoked electrophysiology recordings. After the animal anesthesia was induced, the isoflurane was reduced to 1˜1.5% to maximize spontaneous activity while still maintaining the mice in an inactive state. MagMatrix connector was then connected with the implanted probe for data recording. Electrophysiology data were recorded in a dark room first during spontaneous conditions and then the visually evoked activity was recorded with time-stamped visual stimuli presented with an LCD monitor to the eye contralateral to the implant. Same visual stimulation protocol as the acute mice recording were adopted. For each week, 5-minute spontaneous recordings were performed followed by 7-8 minutes of recordings under visual stimulation. After the recording session, MagMatrix connector was then detached from probe. The probe was then secured to the chamber wall using copper wires and the chamber top was sealed tightly using medical tapes before placing animals back to the cage.

The electrochemical impedance of ROSE probes was measured through the built-in impedance measurement function in RHX recording software (Intan technology). In bench testing, a threshold of 500 kΩ at 1 kHz was used to define functional electrodes (channels). In the brain, the situation is more complicated. This example adhered to three criteria to exclude bad channels. 1) The channel impedance exceeds 10 MΩ in brain. 2) The root-mean-square (RMS) noise in the channel is higher than 20 μV. 3) The recorded LFP coherence with a known-good, neighboring channel is significantly lower or higher than expected. If all three criteria are met, the channel will be excluded from the functional channel group. In this example, SU yield is defined as number of channels that recorded at least one well-isolated SU divided by the number of functional channels. In long-term recordings, the number of functional channels at week 0 were always adopted for the calculation.

Raw data was filtered to 1-300 Hz band for LFP analysis. LFP analysis in this example are done with custom python scripts, including the LFP spectrogram, LFP plot, current source density (CSD) plot, etc. 1D CSD data are derived by the second derivative approximation of the LFP data recorded in a single shank, while 2D CSD data are derived from averaged LFP data in 1-second duration on the same electrode plane. Bicubic interpolation was applied to the CSD data for visualization purposes.

The electrophysiology data was pre-processed with custom Python scripts. Data were filtered to 300-6000 Hz band and after common median referencing prior to spike sorting. The spike sorting was performed using MountainSort 5, with actual ROSE channel coordinates mapped to each channel of data. Sorting scheme 2, training duration=60 seconds, phase1 detection radius=150 μm, phase2 detection radius=50 μm, detection threshold=5.5 were adopted for all the sorting process. After receiving the sorting result, the sorted unit clusters were manually curated by the following criteria: Firing rate>0.1 Hz; Inter-spike-interval (ISI) violation (<2 ms)<1.5%; Full-width half maximum (FWHM) of the mean waveform between 0.15-0.75 ms; Peak-to-valley time (PVT) of the mean waveform between 0.15-0.85 ms; Median spike SNR>4. After applying these criteria simultaneously, the auto-correlogram of each curated unit was visually inspected to exclude any clusters that does not adhere to the expected spike refractory period. Lastly, a unit similarity measure was conducted to prevent over-counting of units from shunted electrodes (See the SU similarity measure and SU tracking). The clusters that did not pass the entire curation process will be counted as MUs. All SU analyses used the curated spike clusters as described above.

SU similarity was calculated using Mahalanobis distances (DM) between the centroids of SU clusters, a measure based on the vector components of spike waveforms and considers the covariance along each dimension. For each recording, within-session, across-unit similarity was first calculated between all curated SU pairs. If two units from the same recording session have unit similarity significantly smaller than 1 (having very similar spike waveforms), the Pearson's correlation coefficient was calculated between their auto-correlograms. If the correlation is larger than 0.75, only one unit with larger spike amplitude will be kept for further analysis. The data from all recording sessions were combined to form a distribution. Then, a 5th percentile of the distribution was used as a threshold to identify stable SUs recorded on the same electrode across sessions.

SU spike count in a bin from 50 ms to 550 ms after stimulation onset were used for the orientation studies. Data from the same angle of stimulation (60 trials per angle) were averaged. Then, orientation selectivity index (OSI=(R_{pref}−Rₒᵣₜₕ)/R_{pref}) and direction selectivity index (DSI=(R_{pref}−Rₒₚₚₒ)/R_{pref}) were calculated for each unit, where R_{pref} stands for the response (mean SU count) to the preferred angle; Rₒᵣₜₕ stands for the response to the angle orthogonal to the preferred angle; Rₒₚₚₒ represents the response to the angle 180° from the preferred angle. After the calculation, the data was summarized in terms of preferred angles and orientation selectivity of each neuron.

A neural encoding model (CEBRA) was used to train spike data from 2 consecutive 500-ms bins after stimulation onset. 83% of data (1200 stimulation trials) were used for training and 17% (240 trials) were saved for testing. The model adopted is: model_architecture=‘offset10-model’, time_offsets=10, batch_size=512. Training labels were set as 0 to 23, representing the 24 different angles. Spike data were standardized before training. For decoding, the K-nearest-neighbors (KNN) algorithm was adopted, with optimal k searched between 1 to 20. When assessing the decoding performance, the mean angle error of all predictions was first calculated. Then, the percentage of predictions that falls into 0 and 15° margin were derived. All the analysis was conducted in Python.

All statistical analysis was performed in Python. For characterizing the evoked recording yield, a Student's paired t-test was conducted, comparing the spikes count (SU/MU) and gamma band power (30-100 Hz) in a 550 ms bin size, 50 ms before the onset of stimulation and another 550 ms bin size, 50 ms after the onset of stimulation. If the p-value resulting from the paired t-test is smaller than 0.05, the statistical significance of evoked responses was concluded. For assessing the recording stability across weeks, one-way ANOVA on the spike amplitude data recorded from each week was performed, with the hypothesis that the mean spike amplitude stays the same across weeks. A p value>0.05 as no significant difference across weeks was then considered. For testing whether the 5th percentile of unit similarity distribution exceeds 1 for all recording sessions, a one-sided binomial test with Bonferroni correction was conducted, and considered p<0.05 for the statistical significance. For evaluating the neuron density and Iba-1 intensity surrounding ROSE and Si probes, two-way ANOVA was conducted with Sidak's multiple comparison, and considered p>0.05 as no statistical significance between 2 groups.

### Example 2

The following example provides a description of embodiments of the ROSE probe as disclosed herein as a monolithic three-dimensional neural probe from deterministic rolling of soft electronics and a description of in vivo validation.

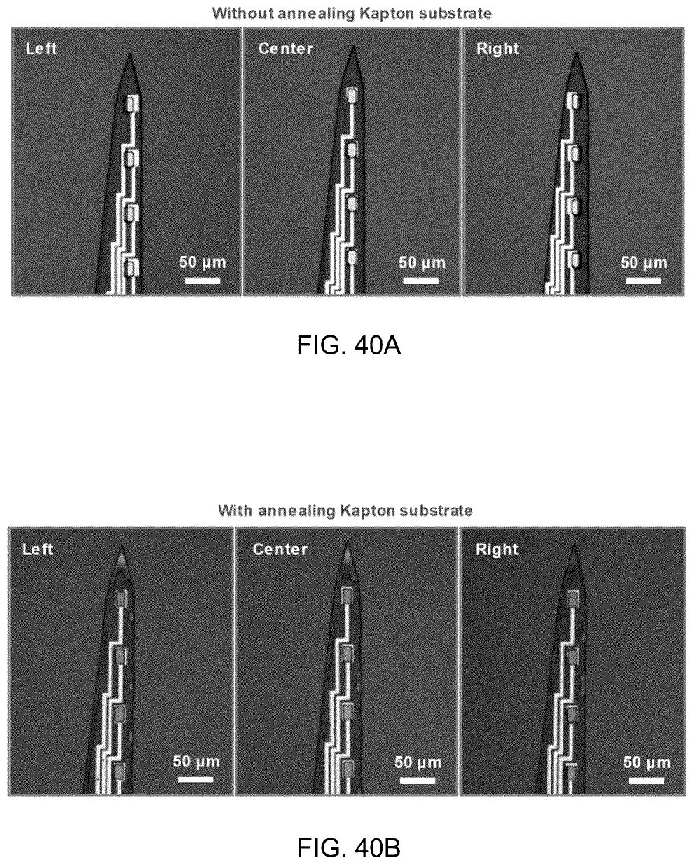

Except for the specific layout of the electrode shanks and interconnects, the design and fabrication of the planar component and shanks is rather conventional (FIGS. 7A-7B and 40A-40B). FIG. 40A displays microscope images in the left, center, and right areas of the planar MEA, showing the misalignment issue of SU8 encapsulation happened in the fabrication. The shrinking of the substrate happened in the procedures with high temperatures (such as ion milling and Ni deposition). FIG. 40B displays microscope images of the shanks after solving the misalignment issue by baking Kapton substrate at 250° C. for 1 hour before the fabrication. As shown in FIGS. 40A-40B, the thermal treatment of the Kapton substrate aids in the alignment of features within the ROSE probe after rolling, as opposed to misalignment without the thermal treatment.

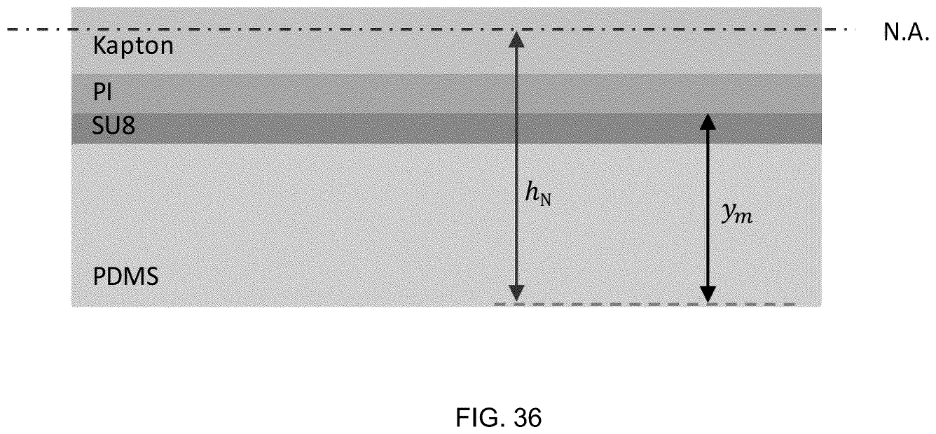

As shown in FIG. 36, an embodiment of the ROSE probe may include four laminates of materials with different Young's modulus and thickness.

FIGS. 4A and 4L-4O displays results of intracortical recording using large-scale ROSE probes results in high-yield single-unit spikes in the rat brain. FIG. 4A (left) illustrates a ROSE probe (32 shanks, 4 electrodes per shank) implanted into a rat visual cortex. FIG. 4A (right) displays photographs of different stages in the insertion process of the ROSE probe. The scale bars are 0.5 mm. FIG. 4L displays the broad-band neural signals (0.1-9000 Hz) recorded from the ROSE probe. Representative data from 2 shanks are shown and the layout of the single shank design is displayed on the left with electrodes labeled from A to D from the tip of the shank. Single-unit spikes were detected and sorted, shown on the right (black lines show the averaged spike waveform of each single unit). FIG. 4M displays the spike firing rate at 4 different insertion depth (electrode A-D). Electrodes in position ‘D’ show the highest spike firing rate. Black data points are the averaged firing rate from all the electrodes at each insertion depth. FIG. 4N displays a local field potential (LFP) spectrogram and spike raster plot of a 9-s recording with 2 trials of visual stimulations included, demonstrating 59% single-unit spike recording. FIG. 4O displays peristimulus time histogram (PSTH) of average spike firing rate in all 113 functioning channels during 4 different trials of visual stimulation. The dashed line shows the time period of visual stimuli.

To validate the in vivo functionality of the ROSE probe, embodiments of the ROSE probes were implanted and tested in the visual cortex of the Long Evans rats, as shown in FIGS. 24A-24B (scale bars in the main figures: 1.5 cm, inset: 1.5 mm). An embodiment of the ROSE probe (128 channel with 32 shank and four electrodes per shank) was manually inserted into the visual cortex after removing the dura (FIG. 4A). An embodiment of the ROSE probe may have a lateral dimension of ˜ 2 mm diameter, to cover the rat's entire primary visual cortex (V1: 1.3-1.5 mm). The electrodes may occupy a total vertical distance of approximately 340 μm, settling into Layer V of the brain's cortex. Both the spontaneous and visually evoked activities were recorded. The in vivo impedance measurement in the rat brain confirmed 113 working channels (88% yield, impedance<1 MΩ at 1 kHz). On average, these working channels recorded electrophysiology data with a root-mean-square (RMS) noise of approximately 7 μV, recording of both local field potential and spiking activities, as shown in FIG. 25, which displays different frequency bands of recorded signals from the rat brain.

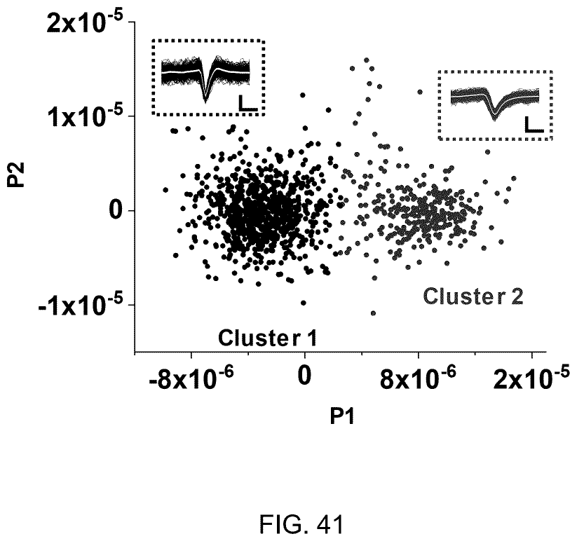

The spike recording capability is one of the most important factors for assessing neural probes. Neuronal spikes recorded by the ROSE probe were reported with two primary classifications: multi-unit (MU) and single-unit (SU) spikes. MU spikes cover the entire threshold crossing events in the high-frequency band (300-6,000 Hz), while SU spikes are derived by sorting the MU spikes using principal component analysis (PCA), as displayed in FIG. 41. Specifically, FIG. 41 displays PCA analysis of a single channel. The inset displays spike waveforms from 2 different clusters, and the scale bar is 50 μV (vertical), 1 ms (horizontal).

Single units from various depths of the visual cortex were identified (FIG. 4L). Overall, the ROSE probe achieved a 67% MU yield and 59% SU yield out of 113 functional channels, competitive when compared to state-of-the-art spike recording performance from contemporary neural probes.

This high performance is notable, especially considering the large channel count and high shank number of the ROSE probe relative to the size of the rodent brain. For example, when compared to recordings in rodents using 3D probes from stacking, a greater SU yield was demonstrated at a much higher shank density. By averaging the spike firing rate recorded from microelectrodes at each of the four different depths (electrode A-D, respectively), it was noticed that D electrodes have the lowest firing rate (FIG. 4M), consistent with the fact that they are at the interface between Layer IV and Layer V, where spike activity is less frequent. In contrast, the highest spike firing rate was observed at the tip of the shank by A electrodes, which can be explained by the location of those electrodes at the mid Layer V41.

Next, the visually evoked responses in anesthetized rats recorded by the 3D ROSE probe were quantitatively studied. Focusing on a single channel (electrode A, shank 23) as an example, the visually evoked response (FIG. 4N) was recognized. Local field potential (LFP) spectrogram (0-200 Hz) for this specific channel showed increased power spectra density at approximately 60-75 Hz when the visual stimuli were on, presumably due to the gamma oscillations elicited by the visual stimulation. MU spike raster plot of signals from this channel also demonstrated the same trend, as spiking activity is more frequent when the visual stimulus is present. By averaging the spike firing rate recorded from all 113 electrodes, clear overall change of spike firing rate under the visual stimulation was observed, consistent with the single-channel result (FIG. 4O). The latency between stimulation onset and the initiation of evoked responses could also be observed to be 50-100 ms. The MU spikes counted in 2 bins (550-ms bin size) were compared, each at 50 ms before and after the visual stimulus onset (paired t-test within channels, channels with p<0.05 counted as visually evoked), to verify the visually evoked response and understand the spatiotemporal dynamics of neuronal response from visual stimulation. Overall, the ROSE probe achieved a 63% evoked MU yield in the 10-minute evoked session, suggesting the implanted regions were highly visually sensitive despite with 32 shanks.

Embodiments of the ROSE probes are monolithic devices that achieve simultaneously layer-specific and multi-regional spike recordings. In previous studies, shank-based neural probes are typically restricted to mapping spike activities within a linear or cross-sectional region of the brain. To capture higher dimensional information, multiple insertion sites with several independent probes are needed, but often with low probe density and requiring careful alignment and accurate electrode localization. Another way of realizing multi-regional recording is to assemble multiple planar probes together. However, it requires the stacking of multiple individual probes and their connecting PCBs. Meanwhile monolithic devices such as the Utah array (UEA) have demonstrated compelling simplicity and practicality and are much ahead in regulatory approval for human studies. But UEAs have a lack of depth profiling and also have been limited by their probe density and array sizes due to their large invasiveness to the brain. For example, UEAs used for rodents are usually with only 4×4 channels.

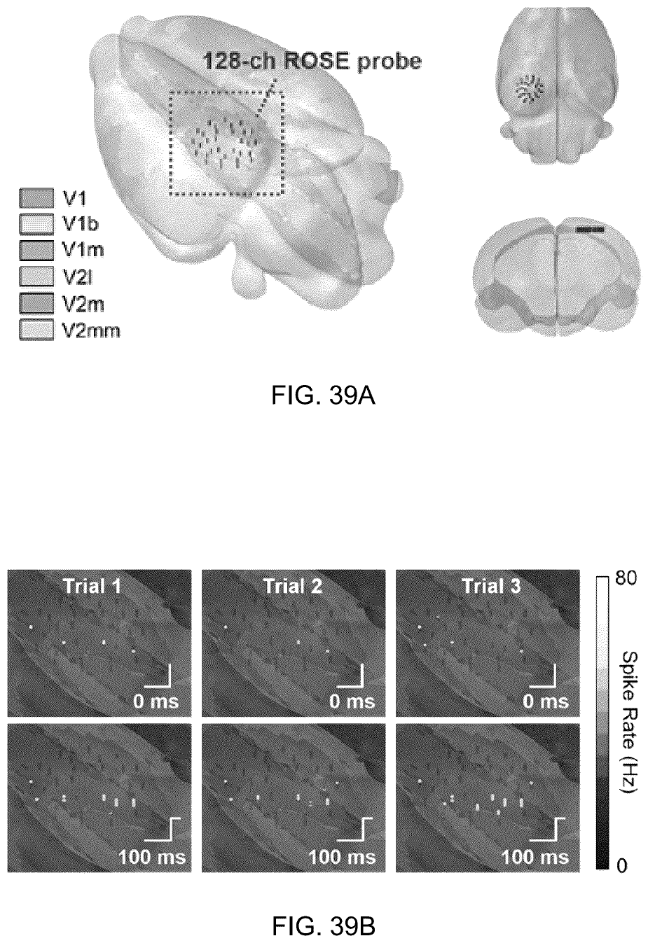

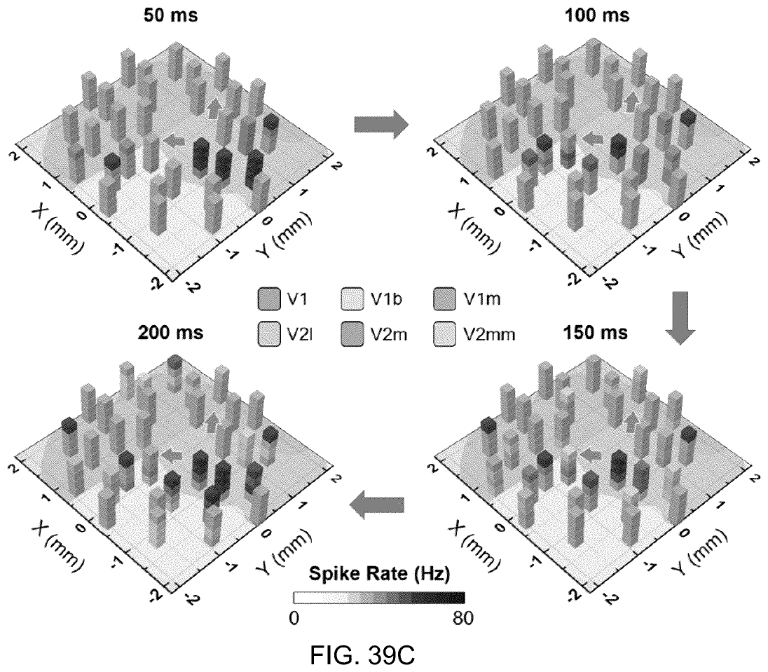

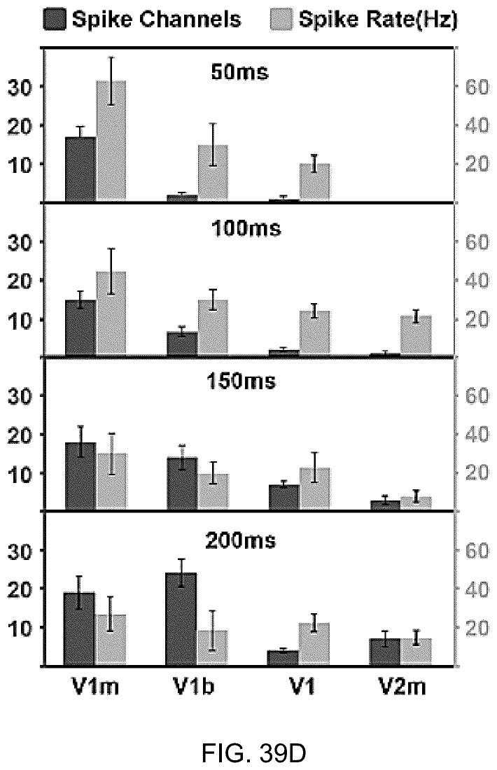

In comparison, because of the monolithic and softness nature, the 128 high-density electrodes in the ROSE probe were implanted all at once and covered a wide portion of the rat visual cortex (˜ 4 mm probe diameter) at Layer IV and V across multiple brain regions while demonstrating high yield in vivo recording performance. The 3D electrode configuration of the ROSE probe clearly displayed the spatially resolved spiking activities in different cortical layers and spanning multiple visual cortical regions (V1m, V1b, V2m). According to the position of the implanted probe, the recorded spike activities were mapped onto the visual cortex of rat, as shown in FIG. 39A. Mapping of spike firing rate in the brain revealed a significant increase of spiking activities in V1 after the stimulation onset, consistent in multiple stimulation trials, as shown in FIG. 39B. Specifically, microelectrodes implanted into the monocular area of V1 (V1m) showed the most robust evoked spiking activity at the onset of visual stimulation while the increase of spiking channels and spike firing rate in other visual cortical regions as the stimulation continued were observed, as shown in FIG. 39C.

This observation was confirmed with the characterization of spiking channels and spike firing rate in each visual cortical region by counting the MU spikes in a 50-ms bin centered at each time point (50, 100, 150, 200 ms) after the stimulation onset, as shown in FIG. 39D. This phenomenon is consistent with the observation made previously by voltage-sensitive dye imaging that visually evoked events are usually initiated from V1m and then propagate both directions to the V1 binocular area (V1b) and V2. To some extent, embodiments of the ROSE probe have demonstrated microscopy-like 3D spatiotemporal mapping of spike activities. The spatiotemporal dynamics of evoked responses (with 10 ms resolution) may also be tested and observed. The results indicate the potential use of the ROSE probe to enable high spatial-temporal resolution in three dimensions and multi-region hierarchical processing of neural activity.

In addition to the rodent models, embodiments of the ROSE probe (256-channel with 32 shank and eight electrodes per shank) were tested in the visually responsive cortex (V4/Inferior Parietal Lobule) of a rhesus macaque monkey (FIG. 5A and FIG. 24B (scale bars in the main figures: 1.5 cm, inset: 1.5 mm)). Similar analyses to the rat data suggested neural signals in different frequency bands (FIG. 25). To summarize, the ROSE probe accomplished a 69% MU yield and 44% SU yield out of the 223 functional channels in the 20 minutes of recording. Although SU yield is usually smaller than the MU yield, a substantial difference between SU and MU yield were found here, and this discrepancy could be mainly due to the lower spatial probability of finding a soma at the tip of the shanks (FIGS. 33A-33B), that is, it was hypothesized that the tip of the shanks reached the bottom of the visually responsive cortex, and electrodes there entered the white matter. It was noticed that when deriving SU spike waveforms using PCA analysis, the first three electrodes from the tip of shanks (microelectrode A-C) also have a low percentage of well-isolated single units, confirming that those microelectrodes are not close to the neurons' somas. When excluding the microelectrodes A-C, the SU yield increases to 58% (82 out of 142 functional channels), while the MU yield remains similar (70%).

Embodiments of the ROSE probe also demonstrated high performance from recording evoked single-neuron activity in the monkey's brain. Evoked MU yield was also characterized for the monkey recording using the same methods mentioned in the rat in vivo. Evoked LFP spectrogram and MU spike raster plot show the visually evoked responses recorded in a single channel similar to the rat in vivo results (FIGS. 5F and 32B). FIG. 32B displays evoked LFP recording from rat brain under 2 trials of visual stimuli. FIG. 5F displays a spike raster plot of monkey recording.

FIG. 5G displays 70 exemplar single units identified in a 5-minute recording from the evoked session. Here, the 256-ch ROSE probe achieved 61% evoked MU yield after calculation, consistent with visual responsiveness of V4/IPL. Hence, in vivo electrophysiology recordings in both the rat and the monkey brains demonstrate the ROSE probe's reliable, high-quality SU recording capability. While the recording in this study is acute, it is believed that the MU/SU yield can be further increased during chronic recordings, as spike yield usually peaks weeks after the implantation.

Embodiments of the present disclosure offer a method to directly transform conventional, planar soft electronics into monolithic 3D neural probes by deterministic rolling. Detailed insertion studies showed all mm-length-scale electrode shanks of the ROSE probe could penetrate the brain without any temporary stiffening and revealed the slipping induced insertion failure mechanism, providing guidelines for ROSE probe design and fabrication. Insertion and recording in both rodent and NHP models further validated ROSE probes with high in vivo performance. Due to its simplicity and non-destructiveness, the ROSE method is applicable to advanced flexible electronics from sophisticated complementary metal-oxide-semiconductor technologies to achieve large-scale integrated 3D probes, with thousands and tens of thousands of electrodes. Incorporating active electronic circuitry in the shanks may be beneficial because of the non-strained nature of these probes after rolling. Further, it is possible to achieve optoelectronic-shank-based 3D probes, enabling multimodal, closed-loop brain interrogation from combining electrophysiology and optogenetics.

Embodiments of these monolithic 3D soft ROSE probes can enable several neuroscientific advances through microscopy-like spatiotemporal spike activity mappings. For instance, in brain areas such as the prefrontal cortex, neurons are connected over many mm, both parallel and perpendicular to the cortical surface. Embodiment of the ROSE probes will enable the study of the relationship between deep layer neurons in one patch of cortex with superficial layer neurons in another patch of cortex. Moreover, it is expected that the three-dimensional nature of recordings will allow better brain computer interfaces by sampling neural activity from both superficial and deep layers of cortex, and/or brain regions which are within sulci, and moving beyond planar Utah arrays that can only sample activity in superficial layers of the cortex.

In embodiments, the shanks of the ROSE probe can be soft. This property can be further exploited, especially for chronic neuroscience studies and clinical applications such as neuroprosthetics, given that Si probes often degrade or fail under chronic in vivo conditions, in large part due to the mechanical mismatch between the rigid implant and soft brain tissues. The mismatch has also been identified as the potential cause of chronic inflammation to the brain and associated retaliatory immune response. As a result, flexible probes fabricated from polymer substrates, such as polyimide, Parylene C, and PDMS, have been shown to provide better mechanical compliance to achieve more reliable neural interfaces. Noteworthy, even though the ROSE probe is flexible, it was observed in several experiments low-yield brain recordings, especially when the shank width is large (wider than 130 μm), presumably due to the large volumetric displacement during the probe insertion.

The overall design of planar component included 5 layers with Kapton/Polyimide (PI) bilayer as a substrate, Au/PEDOT: PSS bilayer as electrodes, and interconnects (with 6 μm line width and 4 μm gap), and SU-8 as an encapsulation layer. All electrode shanks were designed to have a tapered profile with a tip angle of 34° to facilitate insertion and minimize tissue damage. There were four 10×20 μm² microelectrode sites in each shank, with 100 μm electrode pitch and 450 μm distance from the shank tip. The shank width was 110 μm at the widest point, and the entire length of the shank was 1.5 mm. The overall thickness of each shank was 34 μm. Key fabrication steps of the planar component of the ROSE probe included the following. (i) A Kapton film (25 μm; DuPont) was laminated on a glass slide that was pre-spin-coated with 10:1 PDMS (30 μm; Sylgard). (ii) A PI layer (5 μm; PI-2545, HD microSystems) was spin-coated on the Kapton surface to improve the smoothness, and the Kapton/PI substrate was then cured in N₂ atmosphere at 250° C. for 70 min. (iii) Ti (5 nm)/Au (70 nm) was deposited on the substrate by electron-beam evaporation, where Ti acted as an adhesion layer between the PI and Au layers. (iv) PEDOT: PSS (Sigma-Aldrich) was electrochemically deposited with a current density of 0.2 mA/cm² and deposition time of 60s on the Au layer to form Au/PEDOT: PSS bilayer on the substrate. (v) 30-nm-thick Ni was deposited on Au/PEDOT: PSS bilayer by electron-beam evaporation and photolithography steps then patterned the interconnects and electrodes using Photoresist (PR) S1805. Here, Ni acted as a sacrificial layer for patterning and provided better adhesion with PR than PEDOT: PSS. (vi) An ion milling step etched the un-patterned area to form electrodes and interconnects, followed by inductively coupled plasma (ICP) etching with gentle oxygen plasma for 3 min to remove the residues. (vii) A 30% iron (III) chloride (FeCl3) solution removed Ni by soaking the sample for 30 seconds and then used a swab in acetone to further remove both Ni and PR at the same time. (viii) SU-8 encapsulation layer (4 μm) was spin-coated on the sample with openings on the electrodes. (ix) 300-nm-thick Ni was deposited as a hard mask and patterned the shank profile with PR S1827. (x) An inductively coupled plasma (ICP) reactive-ion etching (RIE) step etched the sample to form the shank profile, and this step was divided into four separate sessions to prevent heat accumulation. (xi) A 30% FeC13 solution removed the Ni mask by soaking the sample for 15 min in a vacuum chamber.

Prior to the rolling, a supporting micro-mandrel was glued on the side further away from the interconnect end of the planar component by medical adhesive (3M Vetbond). A PDMS slab with a 10:1 curing agent ratio was bonded to the base region of the planar component to serve as the spacer among the rolled loops (the right edge of the slab was usually placed under the position of the first shank). The rolling started by slowly rotating the micro-mandrel counterclockwise toward the interconnect end of the planar component. A complete ROSE method process typically took about 5-10 min. A Kapton tape attached to the backside of the probe (FIG. 6A) was used to secure the final ROSE structure. The supporting micro-mandrel in the center of the ROSE probe can be removed by sonication for 20 seconds in acetone if needed (FIG. 6B).

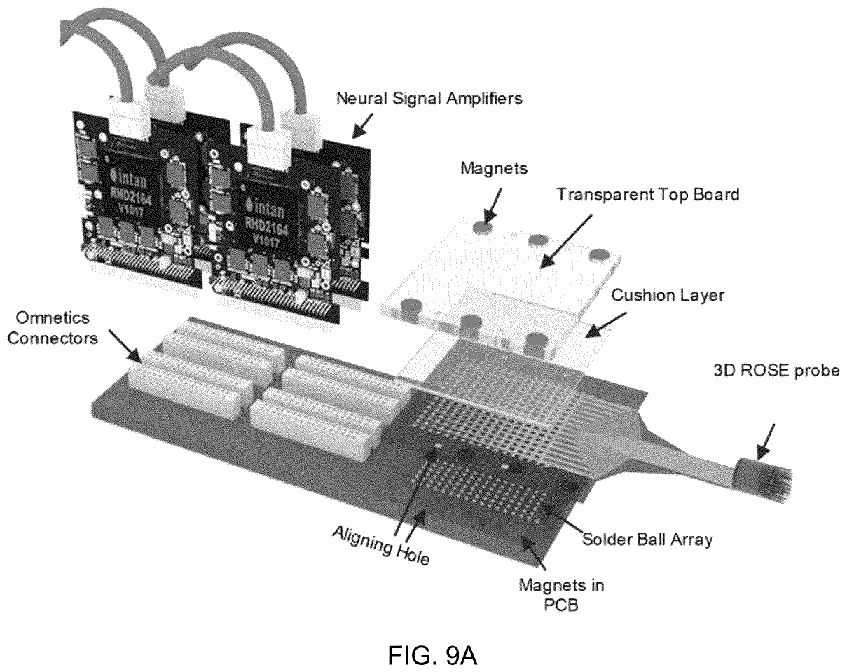

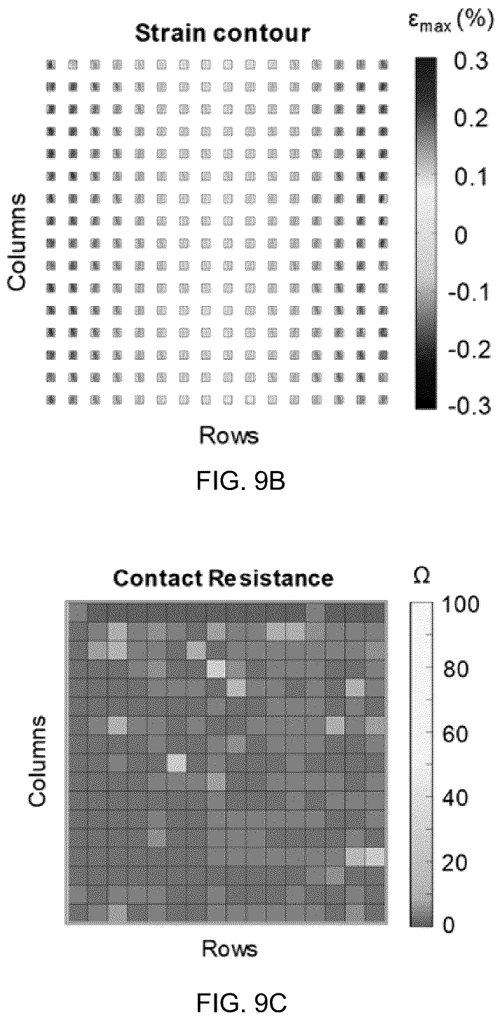

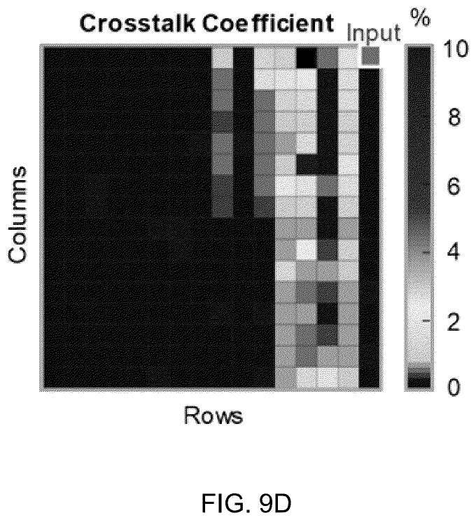

The PCB-based connector interface adopted a matrix of solder balls bumped on the printed-circuit-board (PCB) connector to interface with the thin-film Au I/O pads on the ROSE probe (FIG. 9A). The ROSE probe was first aligned on a polycarbonate top board with a silicone rubber cushion underneath and then pressured onto the solder ball array on PCB with the force of 6 magnets/screws assembled on both boards.

### Example 3

This example provides a description of micromotion simulation of embodiments of ROSE probe.

To reveal the micromotion-induced strain around the implanted probe, the finite element method was used among multiple groups. In this example, a FEA model of 4 shanks distributed in 3D (a subset of 64-shank ROSE probe) was developed to show the micromotion-induced strain around probes of same dimensions but different materials. The specific probe dimensions are 400-μm shank pitch, 400-μm lap pitch, 1.5-mm shank length, 60-μm shank width, 36-μm shank thickness and 30-degree shank tip. The probe materials are polyimide and Si. The 4-shank probe was inserted into brain tissue that is represented by a cube with length of 1.5 mm. The shanks were inserted 1.4 mm deep into the brain media where the probe base was fixed and a displacement was applied to mimic the effect of brain micromotion. Note that the main reason to use four shanks in this study was under a consideration of computational cost. The model can be applicable for larger scale simulation with additional shanks.

To mimic the brain micromotion, only longitude displacement was applied at the boundary of the tissue and the probe was relatively fixed as shown in FIGS. 8A-8B. From the previous experimental investigation, the respiration induced micromotion amplitude was 2˜25 μm with frequency of 1˜2 Hz, and the cardiac rhythm induced micromotion was 1˜4 μm with frequency of 5 Hz. A 6 μm displacement was used to represent the micromotion of brain. The interaction between the shanks and brain tissue was defined as general contact with friction coefficient of 0.2 for the tangential behavior.

To better represent the biomechanical property of brain tissue, an isotropic hyperplastic constitutive model (Ogden model) for cortex was adopted as: μ₁=1.012 kPa; α₁=−16.01; D₁=0.03 kPa⁻¹. For the ROSE probe, both the Kapton (modulus—2.5 GPa, Poisson's ratio—0.34) and Silicon (modulus—179 GPa, Poisson's ratio—0.22) were used in the simulation with same geometry to investigate the effect of materials properties along. The 3D finite element model was established in ABAQUS, both the brain tissue and ROSE probe are deformable. The 8-node linear brick, reduced integration element C3D8R was used for the probe part, and the 10-node quadratic tetrahedron, hybrid element C3D10H was applied for the brain tissue part.

The strain distribution was simulated around the shanks induced by the brain micromotion for both Kapton and silicon (FIGS. 3C-3D). Only the materials properties of shanks were varied in the simulation to investigate the effect of using flexible polymer substrate against conventional rigid silicon substrate. The simulation results indicated that the shanks made with Kapton produce a lower strain field within the surrounding tissue, especially the shank tips. A strain concentrated region was observed along the vertical direction of shanks contact surface with brain due to the shear motion. The region where strain larger than 5% was considered as harmful as a significant cell loss was observed at strain value of 5% in literature. Notably, the strain field visualized in XZ view shown a non-even and localized distribution for different shanks, depending on their relative orientation against the direction of micro-motion. It is a unique feature of ROSE probe due to its spiral distribution, compared to other uniformly distributed 3D probes such as Utah arrays.

As a summary, using flexible substrate such as Kapton, which provides a better mechanical compliance compared to rigid silicon, can be anticipated to improve the long-term stability and biocompatibility by reducing the micro-motion induced damage around shanks.

### Example 4

This example provides a description of the MagMatrix connector designed described herein.

The MagMatrix interface adopted a matrix of solder balls bumped on the printed-circuit-board (PCB) connector to interface with the thin-film Au I/O pads on the ROSE (FIG. 9A). The ROSE probe was first aligned on a polycarbonate top board with a silicone rubber cushion underneath and then pressured onto the solder ball array on PCB with the force of six magnets/screws assembled on both boards. The choice of the magnets and screws depends on the mechanical properties of electrode array substrates. For example, it was found that six magnets achieved 100% low-resistance connections with the PDMS substrate, while six screws were necessary to ensure reliable contact with the Kapton substrate. The cushion layer plays a crucial role in mitigating the stress/strain on the Au I/O pads. It was found that spin-coating approximately 250-μm-thick silicone rubber on the polycarbonate top board yielded the best cushion layer for achieving 100% electrical connection and avoiding the fracturing of Au I/O pads. Additionally, the MagMatrix approach has achieved low and uniform contact resistance across 256 channels, with an even distribution below 20Ω. Moreover, the MagMatrix connector utilizes a non-permanent mechanical contact, making it reusable. Repeated bonding tests have shown no probe yield loss after over 400 times of assembly. The crosstalk of the MagMatrix connector was characterized by inputting test signals (sinewave, 150 Vpp) to one single channel and then measuring crosstalk signals from the other victim channels. Only one channel had the largest crosstalk (˜ 7%), while the crosstalk of other channels was around 1% or lower. Design optimization such as adding ground planes or changing the trace routing of PCB can help to decrease the crosstalk further. In addition, the MagMatrix connector was connected to a stimulation/recording system (Intan Technologies) which was used for impedance measurement at 1 kHz, bench signal recording, and in vivo recording.

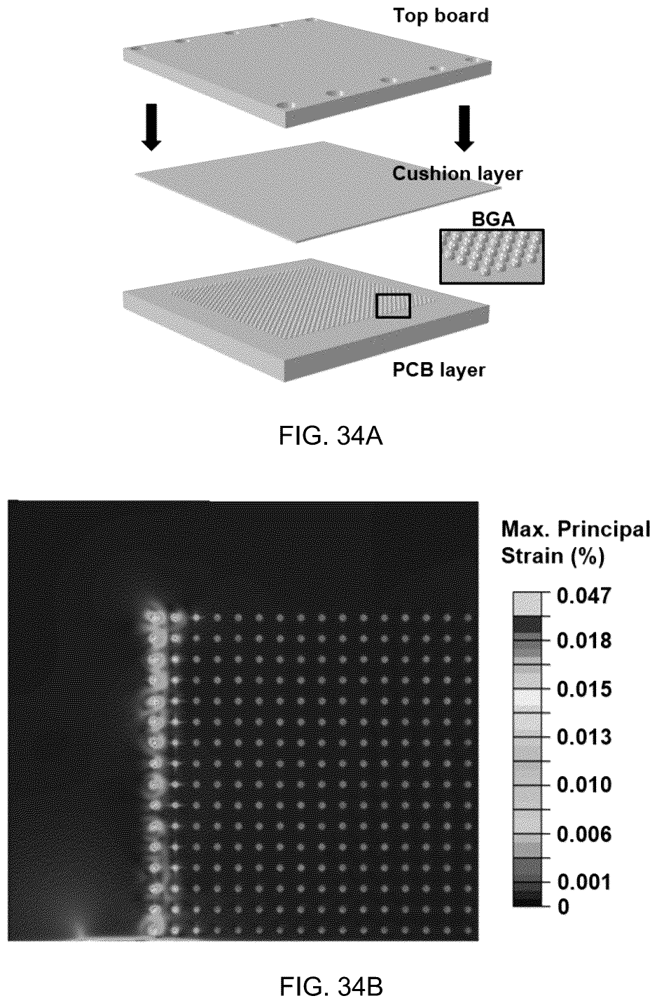

Next, the scalability of MagMatrix connector was investigated. While MagMatrix connectors up to 256 channels (16×16 design) were used in this example, it can be further scaled up to a 32×32 configuration with reasonable size. Based on the PCB design, a 32×32 BGA can be realized with a 10-layer PCB after carefully routing. Based on a design with a pad pitch of 406 μm, it was estimated that the MagMatrix interface would occupy an area of approximately 1.5×1.5 cm², which is within an acceptable range and can be further downsized by decreasing the ball pitch. To explore the reliability of the 32×32 MagMatrix interface, the strain distribution of the 1024-channel pad matrix was simulated by FEA method. Three representative layers were used, including an acrylic top board (deformable, C3D8R), the probe layer with gold I/O pads supported by a cushion layer (deformable, C3D8R), and the BGA integrated PCB layer (rigid), as shown in FIGS. 34A-34B. To save the computational cost, two planes of symmetry which cut the setup into quarters was used. Compression force was then applied from the top board to the bottom PCB. The contact strain was visualized to show the good contact between the solder balls and probes. The maximum strain distribution indicates that there is no fracture of the pads when the contact was made. Thus, the reliability of a 32×32 MagMatrix interface was confirmed.

### Example 5

This example provides a description of methods used for predicting shank positions on the 3D ROSE probe as described herein.

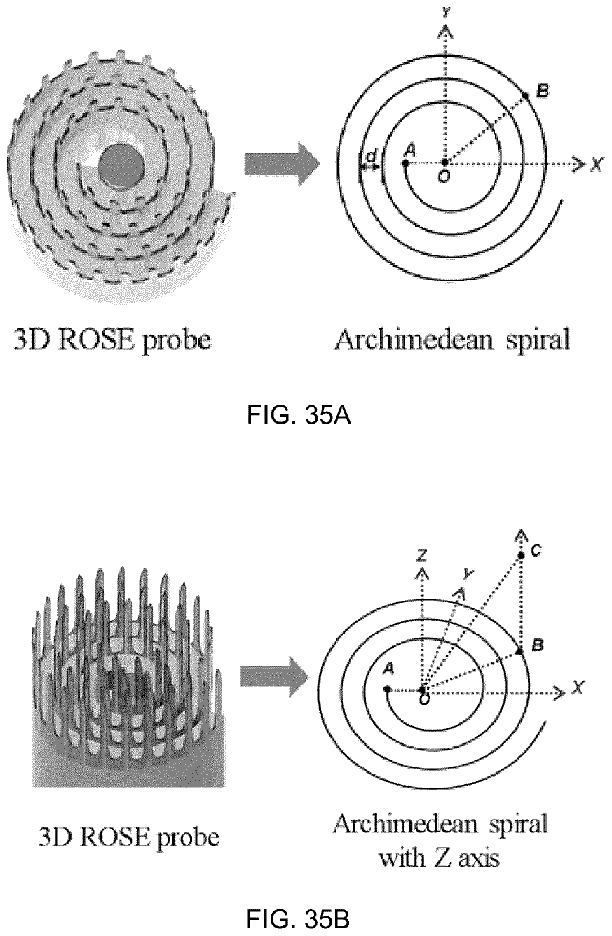

The 3D ROSE probes project to a 2D spiral trace when viewed from the top. Archimedean spiral (also known as the arithmetic spiral) is used to approximately describe the probe positions. It is a plane curve generated by a point moving away from a fixed point with constant radial and angular speeds. In polar coordinates, this spiral trace at the position of the neutral mechanical axis can be described by the equation:

$$r = {r}_{1} + \frac{θ d}{2 π} ,$$

where r is the length of the radius from the center to any given point B (OB), θ is the angular position (amount of rotation from initial rolling point A, θ₁=0), r₁=OA and d is spacing between adjacent layers as shown in FIG. 35A. Specifically, FIG. 35A displays a top view illustration of the 3D ROSE probes. d is the thickness of PDMS spacer and MEA, which controls the distance between loops, OA is the distance between the first shank and the center of the micro-mandrel radius. Point B is an arbitrary shank location.

Denoting A as the position of the first shank, B as that of the n^{th} shank, and the angular position of the n^{th} shank B as θₙ, the arc length (L_{AB}) from Point A to Point B is:

$${L}_{AB} = {\int }_{0}^{{θ}_{n}} rd θ = {r}_{1} {θ}_{n} + \frac{{θ}_{n}^{2} d}{2 π} .$$

Denoting w as the pitch distance between adjacent shanks, L_{AB} equals to the distance between shank A and B before rolling, i.e.:

$${L}_{AB} = ( n - 1 ) w .$$

Solving the above two equations for θₙ gives the angular position of the n^{th} shank as:

$${θ}_{n} = \frac{2}{d} ( \sqrt{{π}^{2} {r}_{1}^{2} + π ( n - 1 ) wd} - π {r}_{1} ) .$$

The radial position of the n^{th} shank is therefore:

$${r}_{n} = {r}_{1} + \frac{{θ}_{n} d}{2 π} .$$

In the X and Y coordinates, the position of Point B can be calculated as:

$$\{ \begin{matrix}{x}_{n} = {r}_{n} cos {θ}_{n} \\ {y}_{n} = {r}_{n} sin {θ}_{n}\end{matrix}$$

The predictions of the shank positions agree with experiments, as is shown in FIGS. 2E-2F and 14A. Note that in real cases, point A is not perfectly overlapped with the position of the first shank (because of process inaccuracy from manual rolling), and hence the distance between point A and the first shank needs to be considered. Furthermore, the electrodes of shank B are aligned with Point C in the Z direction. By designing the planar layout of the ROSE, the X, Y, and Z coordinates (such as point C in FIG. 35B, which has BC length in height) of any electrode for any shank can be determined. Specifically, FIG. 35B displays a side view illustration of the 3D ROSE probe and its geometric picture. Point C is an assumed electrode location in the shank.

### Example 6

This example provides a description of a mechanics model for obtaining the strain in the electrode layer in the ROSE structure.

An embodiment of the ROSE probe may include 4 laminates of materials with different Young's modulus Eᵢ and thickness hᵢ, as shown in FIG. 36 and below: PDMS spacer (E₁=2 MPa, h₁=200 μm), SU8 (E₂=2 Gpa, h₂=4 μm), metal electrodes (not shown in the figure), PI (E₃=2.5 Gpa, h₃=5 μm), and a Kapton film (E₄=2.8 GPa, h₄=25 μm).

The position of the mechanical Neutral Axis (N.A.) h_{N} can be calculated by the equation:

$${h}_{N} = \frac{{\sum }_{i = 1}^{4} [ ( {E}_{i} {h}_{i} / {E}_{1} ) ( {\sum }_{j = 1}^{i - 1} {h}_{j} + {h}_{i} / 2 ) ]}{{\sum }_{i = 1}^{4} {E}_{i} {h}_{i} / {E}_{1}}$$

Denoting the location of the metal electrode layer as yₘ=h₁+h₂, the strain in the electrodes of the n^{th} probe can be obtained by:

$${ϵ}_{m} = \frac{{y}_{m} - {h}_{N}}{{r}_{n}}$$

where rₙ is the radial position of the shank (distance from the neutral axis position of the n^{th} shank to the rolling center) which can be found by the method presented in Example 5. Using this model, the strains in the electrode layers are found and plotted in FIG. 2H right frame. More specifically, with the radius of the first shank r₁=467 μm and pitch w=300 μm from experiments, the strains at the electrode layer for the innermost (Shank #1), middle (Shank #32), and outermost (Shank #64) are found to be −2.82%, −1.38%, and −1.03%, respectively, which agree with finite element analysis results (FIG. 2H (left frame) and FIG. 15A) of −2.66%, −1.19%, and −0.94%, respectively.

### Example 7

This example provides a description of a mechanical analysis of the neck part of embodiments of the ROSE probe.

The ‘neck’ part of the ROSE probe which connects the rolling base and the pad area may be composed of Kapton (25 um), PI (2 um), gold layer (100 nm) with interconnection lines and SU8 encapsulation (5 um). There are potential risks of probe failures if it is under extreme mechanical force during the handling, transportation or recording experiments. As a thin structure, it is flexible and safe to experience a certain degree of deformation. To further validate the reliability of it, the strain distribution in the neck under bending and twisting conditions were revealed via FEA model. The 3D model was established by using the finite element software ABAQUS with C3D8R 8-node linear brick elements with reduced integration and hourglass control. To simple the numerical model, the multi-layers probe was considered dominated by Kapton with Young's modulus of 2.5 GPa and Poisson ratio of 0.34. The setup and boundaries conditions are illustrated in FIGS. 16A-16D. For bending, the left boundary was fixed whereas a 15 mm displacement in −z (−U3) direction was applied on the right boundary (FIG. 16A). For the twisting, the left boundary was fixed whereas an 2π angular displacement around z axis (RU3) was applied on the right boundary (FIG. 16B). The strain distributions were visualized in FIGS. 16C-16D. The neck was bended with dynamic bending radius from infinite (flat) down to 1.12 mm and was twisted up to 180°. The maximum strain in the neck increased with the decrease of bending radius and increase of twisting angle, as summarized in FIG. 43. If considering the fracture strain of gold as 5%, the neck is safe to experience such degree of bending. For twisting, the stress concentration was located at the edge of the neck where there were no interconnection lines, thus it is also safe for the twisting up to 180°.

### Example 8

This example provides a description of mechanics modeling on the insertion of embodiments of ROSE probes into soft gels.

To estimate the penetration force of a single shank, experiments were designed to measure the puncture force of a 10-shank structure with a very large shank pitch of 900 μm (FIG. 22B). The large shank pitch avoids interactions of adjacent shanks during the penetration process such that the 10 shanks penetrate into the gel separately. This example gives an estimated puncture force for a single shank to be 0.46 mN.

The shanks of the ROSE probe contact the gel within a hollow ring area as shown in FIG. 2E-2F. Here it is assumed that although only the shanks are in contact with the gel, the force distribution approximately follows that of a hollow cylindrical punch, i.e.

$$p = c {\sum }_{k = 0}^{∞} {\sum }_{p = 0}^{∞} {( \frac{b}{a} )}^{p} [ {{α}_{pk} ( \frac{r}{a} )}^{2 k} + {{β}_{pk} ( \frac{b}{r} )}^{2 k + 3} ] , b \leq r \leq a$$

where c is a material dependent parameter that accounts for the modulus of the gel, the vertical displacement of the shank and the non-contact area, b is the inner and a is the outer radius of the ring area, with the recursively determined coefficients αₚₖ and βₚₖ.

The position of the n^{th} shank (rₙ, θₙ) can be found by the method in Example 5. Assuming the n^{th} shank redistribute the pressure within the area

$$r ∈ ( {r}_{n} - \frac{d}{2} , {r}_{n} + \frac{d}{2} ) , θ ∈ ( \frac{{θ}_{n - 1} + {θ}_{n}}{2} , \frac{{θ}_{n} + {θ}_{n + 1}}{2} ) ,$$

the force of the n^{th} shank can be calculated as

$${f}_{n} = {\int }_{\frac{{θ}_{n - 1} + {θ}_{n}}{2}}^{\frac{{θ}_{n} + {θ}_{n + 1}}{2}} {\int }_{{r}_{n} - \frac{d}{2}}^{{r}_{n} + \frac{d}{2}} prdrd θ$$

The total force of the array is P=Σₙ₌₁^{N}fₙ, where N is the total number of shanks in the ROSE structure. The puncture force P_{c} is reached when the outermost shank's contact force equals the puncture force of a single shank which is fₛ=0.46 mN. From these equations, the constant c can be determined, and the puncture force of the ROSE probe can be obtained analytically.

Experiments with various values of shank pitch w and PDMS spacer thickness d were performed to measure the puncture forces (FIGS. 21A-21B) and compared to the theoretical predictions as shown in FIGS. 3C-3D. It is clear that the theoretical predictions agree with experimentally measured values of the puncture forces for the ROSE probes.

Consider the above ROSE probe in contact with a large medium of soft gel. The interface work of adhesion is assumed to be γ=0.15 J/m². Assuming the shanks contact the gel in a hollow cylindrical area, this contact loses stability (slippage starts to happen) when the total contact force reaches

$${F}_{c} = {( 1 - {( \frac{b}{a} )}^{m} )}^{n} \sqrt{8 π {a}^{3} E γ} ,$$

where E=30 kPa is Young's modulus of the gel, and the parameters m and n are fitted to be 3.683 and 0.4656, following the previously described method.

The total contact force for all the shanks is obtained from the previous section as

$${F}_{total} ( w , d , {f}_{s} ) = {\sum }_{i = 1}^{N} {f}_{n}$$

which is a function of the pitch between shanks w, the spacer thickness d, the number of shanks N, and the puncture force for a single shank fₛ=0.46 mN. Setting Fₜₒₜₐₗ=F_{c} yields the critical combination of (w_{c}, d_{c}) that indicates the onset of slippage, therefore the scaling map in FIG. 3F (dashed curve) can be obtained. The area above the curve indicates cases where the contact is stable, and the area below the curve corresponds to cases where shanks lose contact stability with the gel and may slip before penetration happens (as illustrated in FIG. 3E). The figure displays cases where the ROSE probe successfully penetrates into the gel and cases in which insertion had problems or failed, which all agree with the theoretical predictions.

### Example 9

This example provides an estimation of Kapton shank buckling forces as described herein.

The buckling force is the maximum force that a shank can withstand before bending and is defined by Euler's formula:

$${f}_{buckling} = \frac{{π}^{2} {I}_{x} E}{{( KL )}^{2}} , {I}_{x} = \frac{{wt}^{3}}{12}$$

where E is Young's modulus, K is column effective length factor, I is the area moment of inertia, L, w, and t are length, width, and thickness of the shank, respectively. It was assumed that the shanks are beams fixed at one side (K=0.7) with a constant cross-sectional area, not considering the tapered tip profile. Assuming E for Kapton is 2.8 GPa, L=1.5 mm, w=110 μm, t=25 μm (only consider the Kapton layer). The buckling force for an individual Kapton shank is 3.58 mN.

As tested in this example, the force corresponding to the insertion of a single shank is approximately 0.46 mN (FIGS. 22A-22B) which is much smaller than the buckling load of the shank, and therefore buckling is not a concern for embodiments of the ROSE probe. When the critical buckling load predicted by the above method and the penetration force is comparable, measures need to be taken to make the shanks thicker/wider to increase the critical buckling load such that the shanks will not buckling before penetration happens.

### Example 10

This example provides a description of tissue volumetric displacement studies in an ex vivo pig brain.

To analyze the potential brain damage caused by a scaled-up version of ROSE probe, three densely packed 256-shank ROSE probes (400-μm shank pitch and 400-μm lap pitch) were implanted into sectioned ex vivo pig brain samples for tissue volumetric displacement analysis. The same insertion protocol used in our in vivo experiments were adhered to, utilizing an automated inserter with an insertion speed of 0.05 mm/s. To harvest enough brain slices with insertion footprints, the probes were inserted 1.8-2 mm into the brain. Embodiments of the ROSE probes were successfully inserted with various shank widths (30, 40 and 55 μm) into the ex vivo pig brain samples without the need for additional insertion aids. Following the implantation, the brain samples were fixed with ROSE probes in place to prevent any further displacements. The fixation process included sequential soaking in Formalin, followed by immersion in 10% and 30% sucrose solutions over a total duration of 96 hours, preparing the samples for slicing.

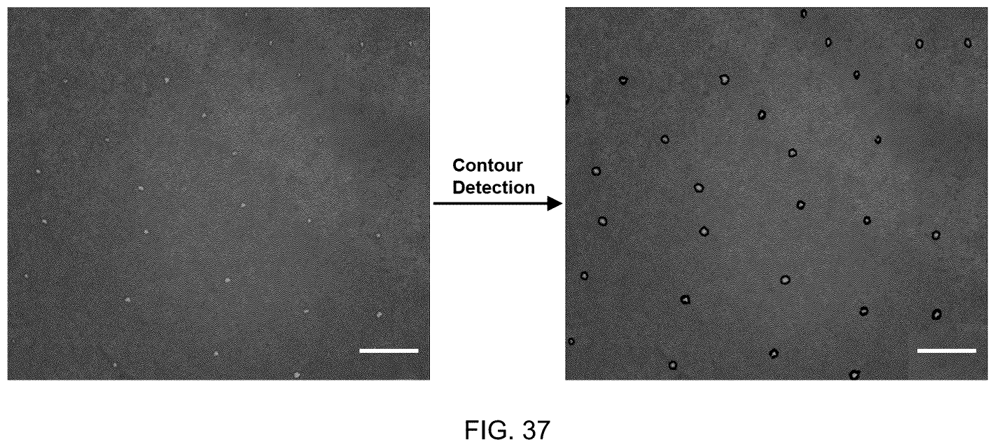

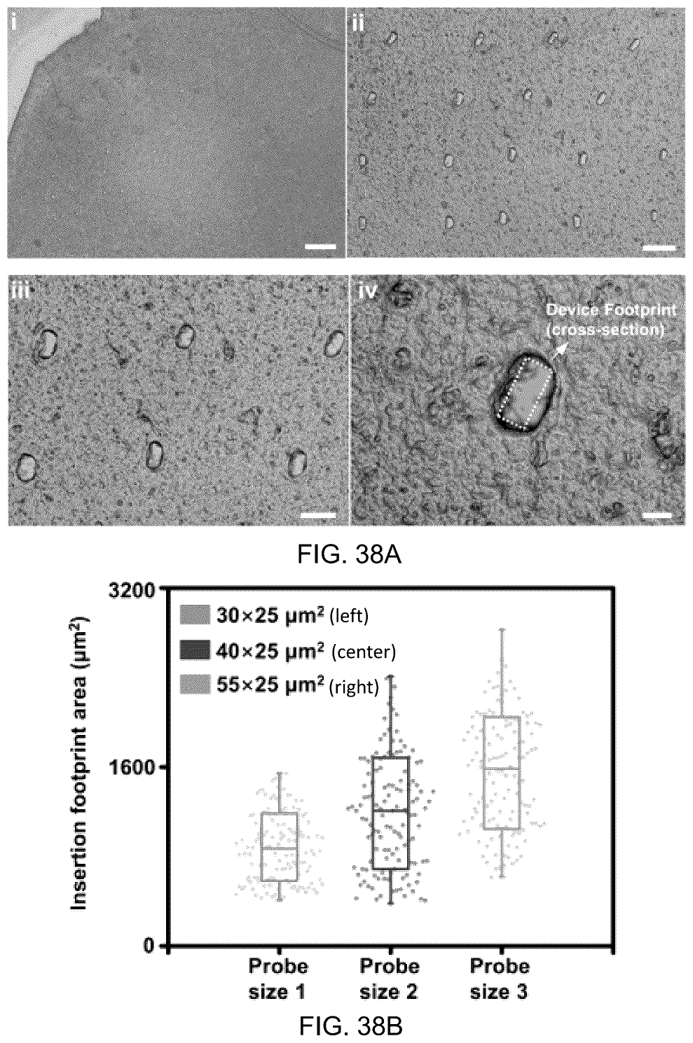

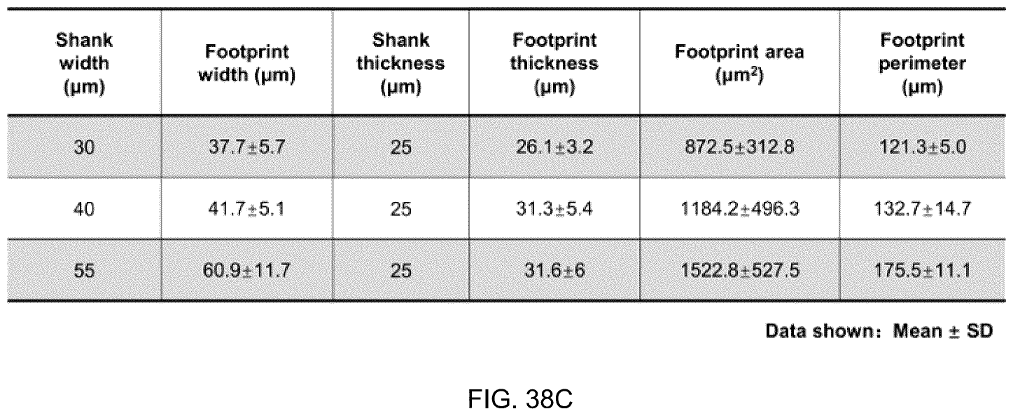

Upon slicing the brain samples, about 130 insertion footprints (holes) in each brain slice were observed, representing about half of the ROSE probe. These footprints exhibited a hole pitch of 356±34 μm and a lap pitch of 415±40 μm (mean±SD), which closely matched the original ROSE design. To conduct comprehensive analysis of the tissue displacements, Python scripts were used to quantify the lengths, widths, area and perimeters of the insertion footprints from the microscopic images (FIG. 37). Imaging analysis revealed that the insertion footprints were comparable to the probe dimensions across varying brain depths, regardless of the shank width used (FIGS. 38A-38C). On average, the area of the insertion holes measured 11-16% larger than the cross-sectional area of the respective probe (N=131 for each width), as summarized in FIG. 38C. The findings indicate that even with the implantation of a dense 256-shank ROSE probe, the resulting insertion footprints remain close to the dimensions of the probe itself. The tissue displacement analysis demonstrates the minimal tissue disruption caused by the implantation of a large-scale, densely packed ROSE probe.

Although the present disclosure has been described with respect to one or more particular embodiments, it will be understood that other embodiments of the present disclosure may be made without departing from the scope of the present disclosure. Hence, the present disclosure is deemed limited only by the appended claims and the reasonable interpretation thereof.

1. A rolling-of-soft-electronics (ROSE) probe comprising:
a planar component comprising one or more shanks; and
one or more electrodes disposed on the one or more shanks;
wherein the one or more shanks are positioned in rows and separated by a space having a pitch; and
wherein the planar component is made of a flexible material and is configured to be rolled.

2. The ROSE probe of claim 1, wherein the planar component further comprises at least one substrate layer and at least one encapsulation layer.

3. The ROSE probe of claim 1, wherein the planar component is rolled such that the one or more shanks are circularly arranged in three-dimensions.

4. The ROSE probe of claim 1, wherein the planar component has a thickness from 1-100 μm.

5. The ROSE probe of claim 1, wherein the one or more shanks comprise a tapered profile with a tip angle of 20°.

6. The ROSE probe of claim 1, wherein each of the one or more shanks has a width from 10-1,000 μm.

7. The ROSE probe of claim 1, wherein each of the one or more shanks has a length from 0.5-5 mm.

8. The ROSE probe of claim 1, wherein each of the one or more shanks has a thickness from 2-100 μm.

9. The ROSE probe of claim 1, wherein the ROSE probe comprises from 10-1,000 shanks.

10. The ROSE probe of claim 1, wherein from 1 to 256 electrodes are disposed on each of the one or more shanks.

11. The ROSE probe of claim 1, further comprising a spacer layer disposed on a base of the planar component and configured to act as a spacer between the rows.

12. The ROSE probe of claim 11, wherein the spacer layer is a polydimethylsiloxane (PDMS) layer.

13. The ROSE probe of claim 11, wherein the spacer layer has a thickness of 100-1,000 μm.

14. The ROSE probe of claim 1, further comprising a connector pad matrix which comprises interconnects of the one or more electrodes.

15. A method of using the ROSE probe of claim 1 as a neural probe.

16. A rolling-of-soft-electronics (ROSE) method comprising:
providing a planar component comprising one or more shanks and one or more electrodes disposed on the one or more shanks;
fixing a micro-mandrel to the edge of the planar component; and
rolling the planar component so that the one or more shanks are circularly arranged in three-dimensions to generate a ROSE probe.

17. The ROSE method of claim 16, wherein the planar component comprises an adhesive portion configured to be secured around the ROSE probe.

18. The ROSE method of claim 16, wherein the one or more shanks comprise a tapered profile with a tip angle of 20°.

19. The ROSE method of claim 16, wherein the ROSE probe comprises from 10-1,000 shanks.

20. The ROSE method of claim 16, wherein from 1 to 256 electrodes are disposed on each of the one or more shanks.
